(19) United States
(12) Patent Application Publication
(10) Pub. No.: US 2026/0151174 A1
(43) Pub. Date: Jun. 4, 2026

(54) SYSTEMS, DEVICES, AND METHODS FOR ABLATION AND DEFUNCTIONALIZATION OF A GALLBLADDER

(71) Applicant: Ictero Medical, Inc., Houston, TX (US)

(72) Inventors: Matthew NOJOOMI, Houston, TX (US); David WATERS, Houston, TX (US); Michael MULANIX, Lakewood, CO (US)

(73) Assignee: Ictero Medical, Inc., Houston, TX (US)

(21) Appl. No.: 19/261,690

(22) Filed: Jul. 7, 2025

Related U.S. Application Data
(63) Continuation of application No. PCT/US2024/010655, filed on Jan. 8, 2024.
(60) Provisional application No. 63/437,483, filed on Jan. 6, 2023.

(51) Int. Cl. A61B 18/02; A61B 18/00; A61B 90/00

(52) U.S. Cl. CPC A61B 18/02; A61B 2018/00029; A61B 2018/00041; A61B 2018/0022; A61B 2018/00267; A61B 2018/00482; A61B 2018/00577; A61B 2018/00821; A61B 2018/0212; A61B 2018/0268; A61B 2090/064; A61B 2218/007

(57) ABSTRACT

Provided herein are catheter devices, systems, and methods to ablate a tissue location. The devises, systems, and methods disclosed herein include ablation systems including a catheter system with inner and outer shafts that deliver an ablation medium (e.g., a cryogenic ablation medium) to a body lumen and evacuate the ablation medium from the body lumen. Devices, systems, and methods disclose herein can include expandable structures that facilitate in positioning of nozzles and/or evacuation of ablation medium from a body lumen. A dehumidification or anti-freezing medium can also be communicated into an internal organ to dehumidify the internal organ before cryoablation.

### CROSS-REFERENCE TO RELATED APPLICATIONS

This application is a continuation of International Patent Application No. PCT/US2024/010655, filed Jan. 8, 2024, which claims priority to and the benefit of U.S. Provisional Patent Application No. 63/437,483 , filed Jan. 6, 2023, and titled, “Systems, Devices, and Methods for Ablation and Defunctionalization of a Gallbladder,” the disclosures of which are hereby incorporated by reference in their entireties.

### TECHNICAL FIELD

This present disclosure relates to devices and methods for ablation and defunctionalization of a gallbladder.

### BACKGROUND

Medical ablation technologies, such as those used in cardiology, oncology, general surgery, gastroenterology, dermatology, and interventional radiology, focus on local tissue targets and, while providing a great degree of ablation depth control, may not be effective or practical for large, high-surface area (HSA) tissue ablation targets within a body. Cryoablation technologies leverage a generic cryogen spray to provide a platform for HSA tissue ablation, but have certain drawbacks associated with safely and effectively delivering energy within closed lumens, such as the gallbladder. For example, ice build-up or other complications during an ablation procedure can lead to injury and/or ineffective ablation. Accordingly, it is desirable to have systems, devices, and methods to address the drawbacks of existing ablation systems.

### SUMMARY OF THE DISCLOSURE

The present disclosure relates to devices and methods for ablation and defunctionalization of a gallbladder. In some embodiments, an apparatus includes a shaft defining a lumen and having a distal portion disposable in a body lumen of a subject, the shaft including: a nozzle disposed on the distal portion, the nozzle defining a plurality of openings in fluid communication with the lumen, the nozzle configured to deliver an ablation medium into the body lumen; and an expandable structure disposed around the nozzle, the expandable structure configured to transition into an expanded state within the body lumen, the expandable structure including a plurality of elongate members that are configured, when the expandable structure is in the expanded state, to (1) position the nozzle from tissue within the body lumen by at least a predetermined distance and (2) allow the ablation medium to pass through the expandable structure to contact and ablate the tissue within the body lumen.

In some embodiments, an apparatus includes an outer shaft having a distal end disposable in a body lumen of a subject, the outer shaft defining a first lumen and a plurality of evacuation openings in fluid communication with the first lumen, the plurality of evacuation openings and the first lumen collectively configured to evacuate an ablation medium from the body lumen, the outer shaft including an expandable structure (1) disposed on the distal end of the outer shaft and (2) configured to transition into an expanded state to surround the plurality of evacuation openings and prevent debris from clogging the plurality of evacuation openings; and an inner shaft disposable within the first lumen and having a nozzle extendable distal to the outer shaft, the inner shaft defining a second lumen in fluid communication with the nozzle, the second lumen configured to deliver the ablation medium to the nozzle such that the nozzle can distribute the ablation medium throughout the body lumen to contact and ablate tissue within the body lumen.

In some embodiments, a method includes transitioning a first expandable structure disposed on a distal end of an outer shaft of an ablation catheter into an expanded state to retain the access sheath within a body lumen of a subject, the distal end of the outer shaft disposed within the body lumen, the outer shaft defining a first lumen; advancing a distal end of an inner shaft into the body lumen via the first lumen, the inner shaft defining a second lumen and including a nozzle and a second expandable structure disposed on the distal end of the inner shaft; transitioning the second expandable structure into an expanded state to position the nozzle at least a predetermined distance from tissue within the body lumen; conveying, via the second lumen, an ablation fluid to the nozzle; and dispensing the ablation fluid from the nozzle such that the ablation fluid transitions into an ablation gas that contacts and ablates the tissue within the body lumen.

In some embodiments, a system includes an outer shaft including a distal end that is disposable within a body cavity; a first inner shaft disposable within the outer shaft and configured to deliver a dehumidification agent into the body cavity; and a second inner shaft disposable within the outer shaft and configured to deliver a cryogenic ablation medium into the body cavity.

In some embodiments, a system includes an outer shaft including a distal end that is disposable within a body cavity; an inner shaft configured to be inserted into the body cavity via the outer shaft, the inner shaft configured to: aspirate fluids from within the body cavity; deliver a dehumidification agent into the body cavity; and delivery a cryogenic ablation medium into the body cavity.

In some embodiments, a method of ablating a body cavity includes delivering, via a first shaft having a distal end disposed within a body cavity, a dehumidification agent into the body cavity, the first shaft being insertable into the body cavity via an outer shaft; after delivering the dehumidification agent, delivering, via a second shaft having a distal end disposed within the body cavity, a cryogenic ablation medium into the body cavity such that the cryogenic ablation medium contacts and ablates tissue of the body cavity, the second shaft being inserted into the body cavity via the outer shaft; evacuating, via the outer shaft, fluids including at least a portion of the dehumidification agent or the cryogenic ablation medium; after the body cavity has thawed to body temperature, repeating the delivering the dehumidification agent, the delivering the cryogenic ablation medium, and the evacuating.

### BRIEF DESCRIPTION OF THE DRAWINGS

FIG. 1 is a schematic illustration of an ablation system (e.g., cryoablation device), according to an embodiment.

FIG. 2 is a schematic illustration of an outer shaft of a catheter system, according to an embodiment.

FIG. 3 is a schematic illustration of an inner shaft of a catheter system, according to an embodiment.

FIG. 4 is a schematic illustration of a catheter system, according to an embodiment.

FIG. 5 is a schematic illustration of a control unit of an ablation system, according to an embodiment.

FIGS. 6A-6B are flow charts of a method for ablation and monitoring thereof, according to an embodiment.

FIGS. 7A-7C are illustrations of different percutaneous and endoscopic access approaches for ablation systems, according to an embodiment.

FIG. 8 is an illustration of an ablation system, according to an embodiment.

FIG. 9 is a detailed view of an outer shaft and an inner shaft of an ablation catheter, according to an embodiment.

FIG. 10 is an illustration of an ablation catheter deployed into a body lumen, according to an embodiment.

FIG. 11 is an illustration of an inner shaft of an ablation catheter with a fenestrated nozzle, according to an embodiment.

FIGS. 12A-12B are illustrations of an outer shaft of an ablation catheter, according to an embodiment.

FIGS. 13A-13C are illustrations of portions of ablation catheters with expandable structures, according to various embodiments.

FIG. 14 is an illustration of an inner shaft of an ablation catheter with an expandable structure, according to an embodiment.

FIG. 15 is an illustration of an ablation catheter with openings for evacuation, according to an embodiment.

FIGS. 16A-16B are cross-sectional views of ablation catheters including evacuation lumens, according to various embodiments.

FIGS. 17A-17B are illustrations of an outer shaft of an ablation catheter with evacuation openings, according to an embodiment.

FIGS. 18A-18B are illustrations of an ablation catheter including an expandable mechanism and evacuation openings, according to an embodiment.

FIGS. 19A-19B are illustrations of an inner shaft of an ablation catheter with a spherical or rounded nozzle, according to an embodiment.

FIG. 20 is an illustration of an inner shaft of an ablation catheter with a spherical or rounded nozzle deployed into a body lumen, according to an embodiment.

FIG. 21 is an illustration of an ablation catheter with a movable nozzle having a free end, according to an embodiment.

FIG. 22 is an illustration of an ablation catheter with a movable nozzle having a fixed end (e.g., fixed using an occluder), according to an embodiment.

FIG. 23 is an illustration of an ablation catheter with a bowed nozzle, according to an embodiment.

FIG. 24 is an illustration of an ablation catheter with a bowed nozzle deployed in a body lumen, according to an embodiment.

FIG. 25 is an illustration of an ablation catheter with nozzle arms, according to an embodiment.

FIGS. 26A-26B are cross-sectional views of a nozzle arm of an ablation catheter, according to an embodiment.

FIGS. 27A-27B are illustrations of an ablation catheter with a spiral nozzle, according to an embodiment.

FIG. 28 is a cross-sectional view of an ablation catheter with a catheter heating system, according to an embodiment.

FIGS. 29A-29B are illustrations of an ablation catheter with a catheter heating system, according to an embodiment.

FIGS. 30A-30B are illustrations of an outer shaft of an ablation catheter and a dilator, according to an embodiment.

FIGS. 31A-31B are illustrations of an outer shaft of an ablation catheter with an expandable structure in an undeployed state and a deployed state, according to an embodiment.

FIGS. 32A-32C are illustrations of an inner shaft of an ablation catheter with an actuator, according to an embodiment.

FIG. 33 is an illustration of an assembled ablation catheter, according to an embodiment.

FIGS. 34A-34B are illustrations of an ablation catheter, according to an embodiment.

FIGS. 35A-35B are an illustration of a sheath of an ablation catheter, according to an embodiment.

FIG. 36 is an illustration of an ablation catheter, according to an embodiment.

FIG. 37A-37B are illustrations of a nozzle assembly, according to an embodiment.

FIGS. 38A-38E are illustrations of a catheter assembly, according to an embodiment.

FIG. 39 is a depiction of temperature sensor placement locations in the gallbladder, according to an embodiment.

FIGS. 40A-40D are illustrations of an ablation system (e.g., cryoablation device) with a pressure sensor, according to various embodiments.

FIG. 41 is an illustration of an ablation catheter including a heated evacuation pathway, according to an embodiment.

FIG. 42 is an illustration of an ablation catheter including a heated evacuation pathway, according to an embodiment.

FIG. 43 is an illustration of a heated evacuation pathway of an ablation catheter, according to an embodiment.

FIG. 44 is a plot of pressure and temperature in the cavity of a vapor load model used to simulate a cavity undergoing cryoablation according to methods described herein, where an ablation device being used to deliver a cryoablation medium to the cavity includes a heated evacuation lumen.

FIG. 45 is an illustration of a dehumidification system for dehumidifying an internal volume of an organ prior to cryoablation of the organ, according to an embodiment.

FIG. 46 is an illustration of an ablation system that includes a catheter assembly for communicating a dehumidification medium and a cryogenic ablation medium into an internal volume of an organ, according to an embodiment.

FIG. 47 is an illustration of an ablation system that includes a catheter assembly for communicating a mixture of a dehumidification medium and a cryogenic ablation medium into an internal volume of an organ, according to an embodiment.

FIG. 48 is a plot of pressure and temperature in the cavity of a vapor load model used to simulate a cavity undergoing cryoablation according to methods described herein, where the cavity started with water (i.e., the cavity was not dehumidified).

FIG. 49 is a plot of pressure and temperature in the cavity of a vapor load model used to simulate a cavity undergoing cryoablation according to methods described herein, wherein the cavity started with about a 4 to 1 ethanol to water ratio (i.e., 25% ethanol).

FIG. 50 is plot of pressure and temperature in the cavity of a vapor load model used to simulate a cavity undergoing cryoablation according to methods described herein, wherein the cavity started with about a 10 to 1 ethanol to water ratio (i.e., 10% ethanol).

FIGS. 51A-51R are schematic illustrations of various operations of a method for dehumidifying and cryoablating a gallbladder, according to an embodiment.

FIG. 52 schematically depicts an aspiration catheter used with ablation systems described herein, according to embodiments.

FIGS. 53A-53B are images of an aspiration catheter used with ablation systems described herein, according to embodiments.

FIGS. 54A-54B are images of a catheter kit, pre-construction and post-construction, according to an embodiment.

FIG. 55 is a flow diagram of a method of administration of a cryogenic fluid to a body lumen, according to an embodiment.

### DETAILED DESCRIPTION

The present disclosure relates to ablation systems, devices, and methods for ablating a body lumen, such as, for example, a gallbladder lumen. In some embodiments, systems, devices, and methods described herein relate to cryoablation devices for tissue ablation. In some embodiments, systems, devices, and methods described herein relate to ablation medium release valves, e.g., for catheter-based cryoablation devices, that are designed to safely, effectively, and uniformly disperse an ablation medium (e.g., a cryogenic ablation medium) onto an area of interest (e.g., tissue lining a gallbladder lumen). In some embodiments, systems, devices, and methods described herein relate to controlling an operation of an ablation system based on sensor data, e.g., pressure and/or temperature data. In some embodiments, systems, devices, and methods described herein include sensors and/or can be used with sensors (e.g., of one or more probes) to track properties or conditions of a body lumen (e.g., a gallbladder lumen) and/or an ablation medium being delivered into the body lumen. Examples of suitable components of ablation systems, including cryoablation devices, are described in International Patent Application No. PCT/US2019/017112, entitled “GALLBLADDER DEFUNCTIONALIZATION DEVICES AND METHODS,” filed on Feb. 7, 2019, and International Patent Application No. PCT/US2020/045436, entitled “SYSTEMS, DEVICES, AND METHODS FOR ABLATION AND DEFUNCTIONALIZATION OF A GALLBLADDER,” filed on Aug. 7, 2020, each of which is incorporated by reference in its entirety.

Gallstones are one of the most common gastrointestinal disorders amongst Americans. Gallstones form when bile, a fluid secreted by the liver and stored in the gallbladder, becomes supersaturated. While they do not cause a problem for many people, gallstones occasionally block the cystic duct, i.e., an outlet of the gallbladder, preventing the gallbladder from emptying. In some instances, the obstruction results in pain, inflammation, and infection. In otherwise healthy patients, the gallstone disease is treated by surgical removal of the gallbladder. However, the risks associated with surgical treatment are considerably higher in certain patient populations. For example, one in five Medicare patients have been shown to suffer an adverse outcome. Non-surgical treatment options for these patients are limited and focus on relieving acute symptoms, without addressing the underlying cause of the disease. In some instances, the disease is likely to recur, resulting in additional clinical risk and significant cost. There currently is no long-term solution for gallbladder disease in high-risk patients.

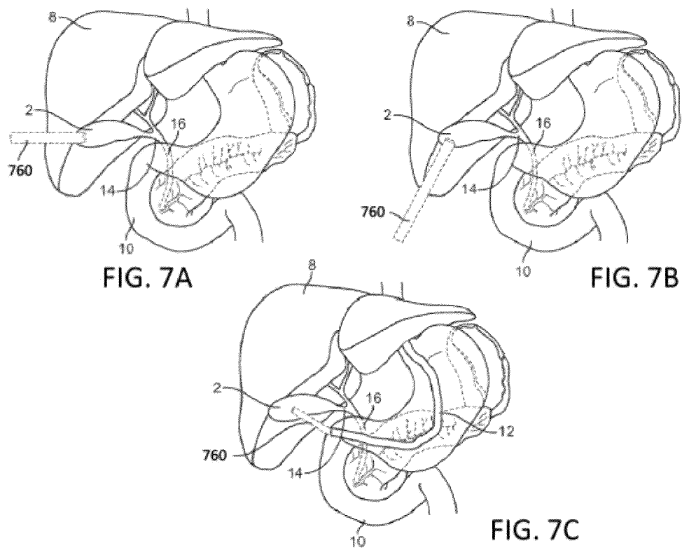

As depicted in FIGS. 7A-7C, the gallbladder 2 is a small hollow organ in the gastrointestinal system. A blind-ended tubular outpouching of the biliary tree, the gallbladder 2 is a pear-shaped organ with a storage capacity of 30 milliliters (mL)—50 mL. The gallbladder is typically 2-3 centimeters (cm) in breadth and 7-10 cm in axial length. The gallbladder is typically divided into three parts: the fundus, body, and neck. The neck contains a mucosal fold, known as Hartmann's Pouch, which is a common location for gallstones to become lodged, resulting in cholecystitis. As shown in FIGS. 7A-7C, the gallbladder 2 opens into a cystic duct 14 and connects to the liver 8 by the common hepatic duct 18 which bifurcates into the right hepatic duct and the left hepatic duct. The gallbladder 2 is connected to the small intestine 10 by the common bile duct 16.

Histologically, the gallbladder has 4 layers, including the serosa (the outermost layer), a muscular layer, lamina propria, and the innermost mucosa layer. The mucosal layer of the gallbladder is the innermost layer of the gallbladder wall and concentrates the bile. The serosa is derived from the visceral peritoneum and covers the anterior fundus, body, and neck of the gallbladder. Inside the serosa, a single muscular layer envelopes the lamina propria. The mucosa that lines the inner lumen of the gallbladder is composed of columnar epithelial cells which secrete mucin and dehydrate bile via the action of multiple ion channels. Occasionally, outpouchings (known as Rokitansky-Aschoff nodules) of the mucosa extend into deeper layers of the gallbladder wall.

The gallbladder stores and concentrates the bile produced by the liver and releases the stored bile into the small intestine, where the bile helps in the digestion of fats in food. Bile is made by hepatocytes in the liver and subsequently secreted into hepatic ductules which coalesce into intrahepatic ducts. These ducts converge to form the right and left hepatic ducts which then combine into the common bile duct. The common bile duct joins with the pancreatic duct just proximal to the ampulla of Vater in the duodenal wall. Bile produced by hepatocytes flows through the biliary system and into the duodenal lumen to aid in digestion.

Flow into the duodenal lumen is regulated at the level of the ampulla of Vater by the sphincter of Oddi. During an unfed state, when bile is not needed for digestion, the sphincter is closed, resulting in routing of bile to the gallbladder for storage. During storage, bile becomes supersaturated, providing a nidus for the formation of gallstones and sludge (very small gallstones). The majority of gallstones are “brown stones,” that are mainly comprised of cholesterol (typically >80%). These stones tend to be brittle and are readily crushed. A minority of stones are predominantly bilirubin (“black stones”; <20% cholesterol) and are often much harder. Mixed stones contain a variable amount of bilirubin and cholesterol.

Mobile gallstones that remain in the lumen of the gallbladder have the potential to cause various pathologies. In some instances, the gallstones become lodged at the neck of the gallbladder, occluding the cystic duct. The lodged gallstones cause gallbladder distension and intermittent right upper quadrant discomfort (likely from intramural muscle spasm at the organ attempts to empty against an increased pressure gradient), a condition known as symptomatic cholelithiasis. In some instances, the gallstones become lodged more permanently at the gallbladder outlet, resulting in inflammation and infection. This is a condition known as cholecystitis, which requires urgent intervention as it can progress to systemic infection.

Alternatively or in combination, gallstones or sludge passes through the cystic duct, becoming lodged in the common bile duct, blocking the flow of bile, resulting in a potentially life threatening condition known as ascending cholangitis. In some embodiments, the debris becomes lodged at the confluence of the pancreatic and common bile ducts, causing stagnation of pancreatic secretions, resulting in pancreatitis (inflammation of the pancreas).

In cholelithiasis, supersaturation of bile in gallbladder leads to the formation of gallstones. In some instances, impacted gallstones leads to inflammation, pain and infection of the gallbladder. When the gallbladder is inflamed, the mucosal layer of the gallbladder becomes more prominent. In some instances, the gallstone disease is diagnosed by ultrasounds or other imaging methods. Provided herein are methods and devices configured to definitively treat benign gallbladder disease in a minimally invasive manner in patients with symptomatic gallstones in order to reduce health care costs and patient morbidity.

Laparoscopic cholecystectomy is a treatment for gallstone disease and is a commonly performed general surgery procedure. During laparoscopic cholecystectomy, small incisions are made in the abdomen, facilitating the removal of the gallbladder with a camera and small instruments. The procedure is safe in otherwise healthy patients, and often does not require hospital admission. In uncomplicated cases, patients are often back to work within two weeks.

In a number of patient populations, the surgical risk associated with laparoscopic cholecystectomy is considerably higher. In some instances, these populations include critically ill patients, patients with intra-abdominal scarring from chronic disease and previous surgery, and elderly patients who tend to have a higher incidence of medical comorbidities. One such population is the Medicare population, which comprises approximately 200,000 laparoscopic cholecystectomies per year in the US. Twenty one percent of these surgeries result in an adverse outcome, including prolonged length of stay and readmission and other perioperative complications. In addition to the direct costs associated with these complications, many elderly patients are at risk of not returning to their baseline level of health, resulting in additional healthcare costs.

There are non-surgical options to treat gallstone disease. These include the administration of antibiotics, or placement of a cholecystostomy tube to drain the gallbladder contents, or a combination of the two. However, the non-surgical options do not provide a long-term solution. These options are effective temporizing measures, and they do not treat the cause of the disease. During a percutaneous cholecystostomy, a cholecystostomy tube is placed through the rib cage into the gallbladder. The percutaneous cholecystostomy can take place in an interventional radiology (IR) suite or at the patient's bedside but does not provide a definite treatment of the gallstone disease. Often times, the non-surgical options lead to recurrence and additional hospitalization costs.

For patients with cholecystitis who have a high risk of surgical complications, the treatment is percutaneous decompression of the gallbladder (via a percutaneously inserted cholecystostomy tube) in conjunction with antibiotics. This treatment provides a temporizing measure to allow the patient to recover from the systemic effects of the ongoing infection (sepsis) and return to their baseline state of health (commonly referred to as “cooling off” by healthcare professionals). The cholecystostomy tube remains in place until the patient has recovered. About 6-8 weeks following placement, a cholangiography by injection of radiopaque contrast through the tube under fluoroscopy is performed to determine if the cystic duct is patent (open). The cholecystostomy tube is removed if the cystic duct is patent. The treatment is interval cholecystectomy as it reduces the rate of recurrence of the gallstone disease. If there is no communication between the cystic duct and the common bile duct, the tube remains in place until cholecystectomy is performed, or patency is demonstrated on subsequent cholangiography. There is no definitive treatment available for high risk patients, placing them at risk for disease recurrence and exposure to the associated clinical risks and healthcare costs.

Ablation technologies have been used to treat other diseases. For example, ablation has been used in treatment of esophageal metaplasia and endometrial hyperplasia. However, ablation technologies are not readily available for treating gallstone disease. Ablation technologies often are applied to a small targeted area, such as a nerve, and are not typically used for applying to a diffuse area or a tissue or organ. Systems, devices, and methods described herein relate to ablating and defunctionalizing a gallbladder, and are specifically designed to safely and efficiently ablate the gallbladder.

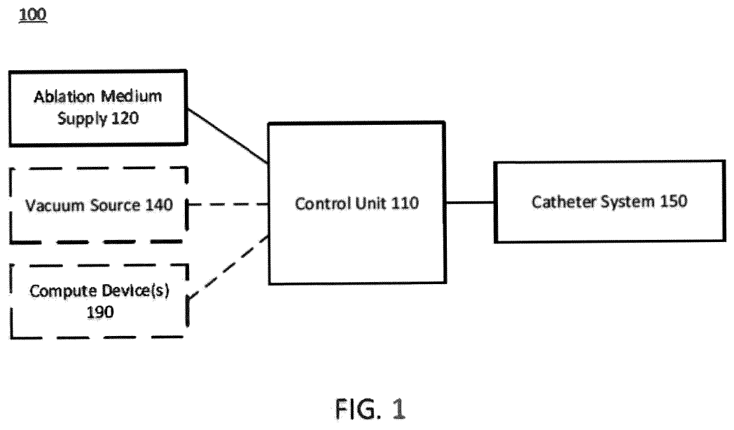

FIG. 1 is a schematic illustration of an example ablation system 100, according to an embodiment. The ablation system 100 includes a control unit 110, a catheter system or ablation catheter 150, and an ablation medium supply 120. The control unit 110 can control the operation of one or more components of the ablation system 100.

The control unit 110 can be operatively coupled to the ablation medium supply 120, which provides a supply of an ablation medium. For example, the control unit 110 can be configured to control delivery of the ablation medium into a body lumen (e.g., gallbladder lumen). In some embodiments, the ablation medium is a cryogenic ablation medium. In some embodiments, the cryogenic ablation medium is a liquid. In some embodiments, the cryogenic ablation medium is a gas. In some embodiments, the cryogenic ablation medium undergoes a liquid-to-gas phase transition when being delivered using the systems and devices disclosed herein. In some embodiments, cryoablation is achieved via the refrigerant property due to the liquid to gas phase change from an ablation medium, such as liquid nitrous oxide, carbon dioxide, and argon. In some embodiments, the cryogenic ablation medium is one or more of nitrous oxide, nitrogen, carbon dioxide, or argon. In some embodiments, the cryogenic ablation medium can transition from a first state (e.g., a liquid) to a second state (e.g., a gas) and increase up to about 600 times an original volume of the cryogenic medium during the transition. In some embodiments, the control unit 110 can control one or more of a temperature, a pressure, etc. of the ablation medium. In some embodiments, an ablation medium such as a cryogenic ablation medium can cool tissue to between about −120 degrees Celsius and about 0 degrees Celsius, including all values and subranges in between, when the cryogenic ablation medium is used with the systems and devices disclosed herein. In some embodiments, the ablation medium supply 120 can be a cryogen cartridge. In some embodiments, the cryogenic ablation medium can be delivered at a predetermined wattage for a predetermined period of time. For example, a cryoablation fluid can be delivered at between about 40 and about 200 Watts, including all values and ranges therebetween. In some embodiments, the cryogenic ablation medium can be delivered for a period of time of between about 10 seconds and about 5 minutes, inclusive of all values and ranges therebetween, including, for example, about 30 seconds.

The control unit 110 can optionally be coupled to a vacuum source 140 (e.g., a vacuum or suction pump, an aspirator, etc.). In some embodiments, the control unit 110 can control the vacuum source 140 to apply a vacuum to a channel or lumen of the catheter system 150, e.g., to remove or evacuate an ablation medium from within a body lumen (e.g., gallbladder lumen). For example, the control unit 110 can activate the vacuum source 140 to apply negative pressure within a lumen of the catheter system 150 to evacuate a portion of an ablation medium, such as a cryogenic ablation medium, that has been delivered to the body lumen. Alternatively, in some embodiments, the ablation system 100 does not include a vacuum source 140, and ablation medium can be evacuated from a body lumen via passive evacuation driven by a pressure differential between an interior of the body lumen and an exterior environment. For example, when an ablation medium such as a cryogenic ablation medium is delivered into a body lumen, the ablation medium can increase pressure within the body lumen relative to an environment exterior to the body lumen (e.g., an exterior atmosphere), and that pressure differential can drive evacuation of a portion of the ablation medium out of the body lumen, e.g., via a lumen defined by the catheter system 150.

In some embodiments, the control unit 110 can include or be operatively coupled to one or more sensors (e.g., pressure sensors, temperature sensors), and can operate or control one or more components of the ablation system 100 based on data collected by the one or more sensors. For example, the control unit 110 can be coupled to a pressure sensor and, based on measurements from the pressure sensor, control delivery of the ablation medium (e.g., from ablation medium supply 120) and evacuation of the ablation medium (e.g., using vacuum source 140) to maintain pressure within a body lumen within a predetermined range of pressures. Stated differently, the control unit 110 can be configured to control insufflation of a body lumen such that pressure within the lumen is maintained within a predetermined range of pressures. In some embodiments, the predetermined pressure range is less than 50 mmHg, or less than 100 mmHg. In some embodiments, the predetermined pressure range is about 0 mmHg to about 40 mmHg, or about 30 mm Hg to about 40 mm Hg. In some embodiments, the control unit 110 can be operatively coupled to one or more valves, which the control unit 110 can control to allow and/or terminate delivery or evacuation of an ablation medium. Examples of suitable valves are described in International Patent Application No. PCT/US2020/045436, incorporated herein by reference.

In some embodiments, the control unit 110 and/or other components of the ablation system 100 can optionally be coupled to one or more additional compute devices 190. Compute device(s) 190 can be any suitable processing device configured to run and/or execute certain functions. The one or more compute device(s) 190 can include, for example, a computer, a laptop, a portable device, a mobile device, or other suitable compute device including a processor, a memory, and/or an input/output device. For example, the control unit 110 can be coupled to a remote compute device, such as a workstation, through which a user (e.g., physician, administrator, etc.) can control one or more operational parameters of the ablation system 100. The control unit 110 and the one or more compute device(s) 190 can be configured to send data and/or receive data from one or more other compute device(s) 190, e.g., via a network. For example, the control unit 110 can send alerts and/or other information to a remote device (e.g., a display, a mobile device) such that the remote device can present that information to a user (e.g., a physician). In some embodiments, the control unit 110 can send data such as patient information, operational status of one or more components of the ablation system 100, etc.

In some embodiments, the control unit 110, ablation medium supply 120, and/or other components of the ablation system 100 can be integrated into a handheld device that is attached to a proximal end of the catheter system 150. The handheld device can include on or more input and/or output devices (e.g. buttons, switches, keyboards, touchscreens, display, etc.) through which an operator of the ablation system 100 can control the operation of the ablation system 100 to perform an ablation procedure. In some embodiments, the control unit 110 can be remote from the catheter system 150, and a remote operator can control one or more components of the ablation system 100 to perform an ablation procedure.

The catheter system 150 can be percutaneously inserted into a body lumen (e.g., a gallbladder lumen) to scar down and defunctionalize portions of anatomy (e.g., the gallbladder) without the need for surgical removal of the anatomy. The catheter system 150 can be used in the interventional radiology (IR) suite and with local anesthesia, eliminating the risks associated with general anesthesia in high risk surgical patients. Placement of the device leverages existing IR workflows and can be deployed in a manner similar to existing devices. For example, for placement in the gallbladder lumen, such placement can be similar to a cholecystostomy tube or percutaneous gallbladder drainage tube.

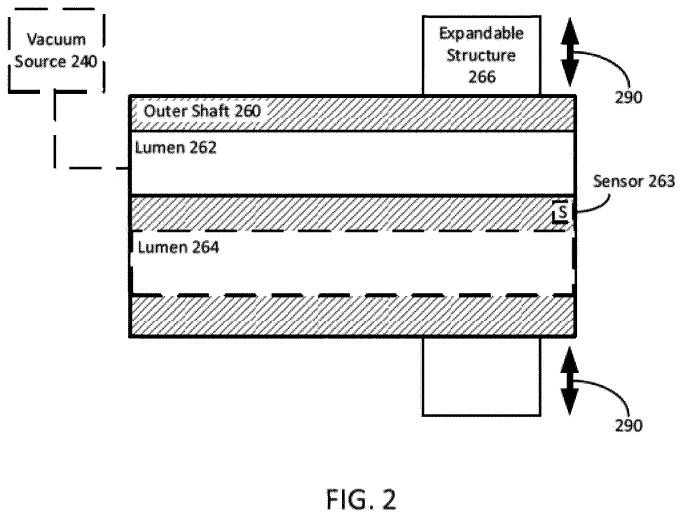

FIGS. 2-4 provide schematic views of portions of an example catheter system 250, according to some embodiments. Catheter system 250 can be structurally and/or functionally similar to catheter system 150. For example, catheter system 250 can be coupled to a control unit (e.g., control unit 110) and/or be configured to receive an ablation medium from an ablation medium supply (e.g., ablation medium supply 120). The catheter system 250 can include an outer shaft 260 and an inner shaft 270. The inner shaft 270 can also be referred to as a first shaft, and the outer shaft 260 can be referred to as a second shaft. In some embodiments, the outer shaft 260 and the inner shaft 270 can be separate components that are used together, e.g., to perform an ablation procedure. For example, the outer shaft 260 can be implemented as an access sheath or introducer, and the inner shaft 270 can be implemented as a catheter that is insertable into a lumen of the introducer. In some embodiments, the outer shaft 260 and the inner shaft 270 can be integrated into a single catheter device, e.g., a device having two concentric shafts.

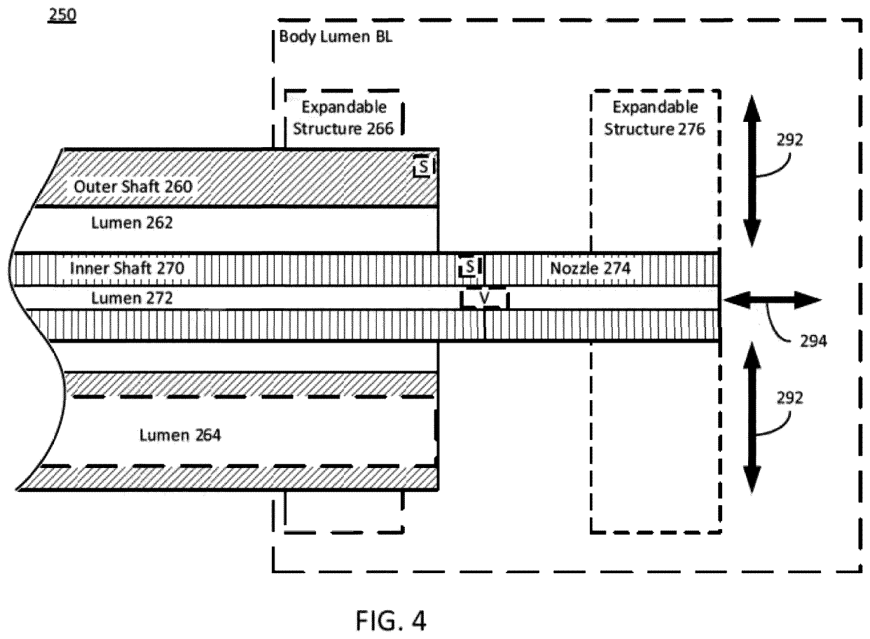

FIG. 2 provides a detailed view of the outer shaft 260. The outer shaft 260 can be, for example, an access sheath. The outer shaft 260 can define a lumen 262. The lumen 262 can be configured to receive one or more instruments, including, for example, the inner shaft 270. The outer shaft 260 can be configured to provide access to a body lumen BL (e.g., a gallbladder lumen). For example, a distal end of the outer shaft 260 can be positioned within a body lumen BL, as schematically depicted in FIG. 4. Once positioned inside the body lumen BL, the outer shaft 260 can enable delivery of one or more instruments into the body lumen BL, e.g., via lumen 262. For example, as depicted in FIG. 4, the inner shaft 270 can be inserted into lumen 262 of the outer shaft 260 and navigated into the body lumen BL such that a distal end of the inner shaft 270 is positioned inside of the body lumen BL.

In some embodiments, the lumen 262 can be configured to evacuate or drain fluids (e.g., liquids or gases) and/or debris (e.g., gallstones or fragments thereof, tissue, etc.) from within the body lumen BL. For example, the lumen 262 can allow an ablation medium (e.g., a cryogenic ablation medium) delivered to the body lumen BL to be evacuated from the body lumen BL. In some embodiments, the lumen 262 can be operatively coupled to a vacuum source 240, which can be activated to apply negative pressure within lumen 262 to evacuate fluid from within the body lumen BL. Alternatively, the lumen 262 can function as a passive evacuation passageway for fluid to exit the body lumen BL. For example, as ablation medium is delivered into the body lumen BL and pressure increases within the body lumen BL relative to an exterior of the shaft 260, such pressure can passively drive a portion of the ablation medium out from the body lumen BL via lumen 262.

In some embodiments, the outer shaft 260 can define one or more additional lumens, e.g., a lumen 264, which can be structurally and/or functionally similar to lumen 262. For example, lumen 264 can also be configured to provide access into the body lumen BL. In some embodiments, lumen 262 can be configured to receive the inner shaft 270 and lumen 264 can be configured to receive a different surgical and/or monitoring device (e.g., a probe, a second ablation device, etc.). In some embodiments, one or more of lumens 262, 264 can be fluidically coupled to a sensor (e.g., a pressure sensor) to allow for pressure measurements of the body lumen BL and/or other portions of the body. For example, a sensor integrated into a control unit (e.g., control unit 110) can be in fluid communication with one or more of lumens 262, 264 and take measurements (e.g., pressure measurements) of an environment within the outer shaft 260 and/or body lumen BL.

In some embodiments, the outer shaft 260 optionally includes a sensor 263. In some embodiments, the sensor 263 can be located in a distal portion of the outer shaft 260 that is configured to be disposed within the body lumen BL. Alternatively, the sensor 263 can be disposed at a different location along the outer shaft 260, including, for example, within a lumen (e.g., lumen 262, 264), at a proximal end at the outer shaft 260, etc. The sensor 263 can be configured to capture information about an environment within the body lumen BL or other environment within and/or surrounding the outer shaft 260. For example, the sensor can be configured to measure a property (e.g., pressure, temperature) of an ablation medium being delivered to the body lumen BL, a property (e.g., pressure, temperature) of the body lumen BL or fluid within the body lumen BL, etc. The sensor 263 can include, for example, a pressure sensor (e.g., pressure transducer, strain gauge transducer, diaphragm displacement sensor, optical fiber pressure sensor, solid state sensor), temperature sensor, light sensors, gas sensors, etc. In some embodiments, sensor 263 can be coupled to a control unit (e.g., control unit 110) and/or other compute device (e.g., compute device 190) via a wired connection, such as, for example, a wire that is coupled to and/or disposed within the outer shaft 260. In some embodiments, the sensor 263 can be configured to wirelessly transmit data, e.g., indicative of one or more measured properties of the body lumen BL, to control unit and/or another compute device.

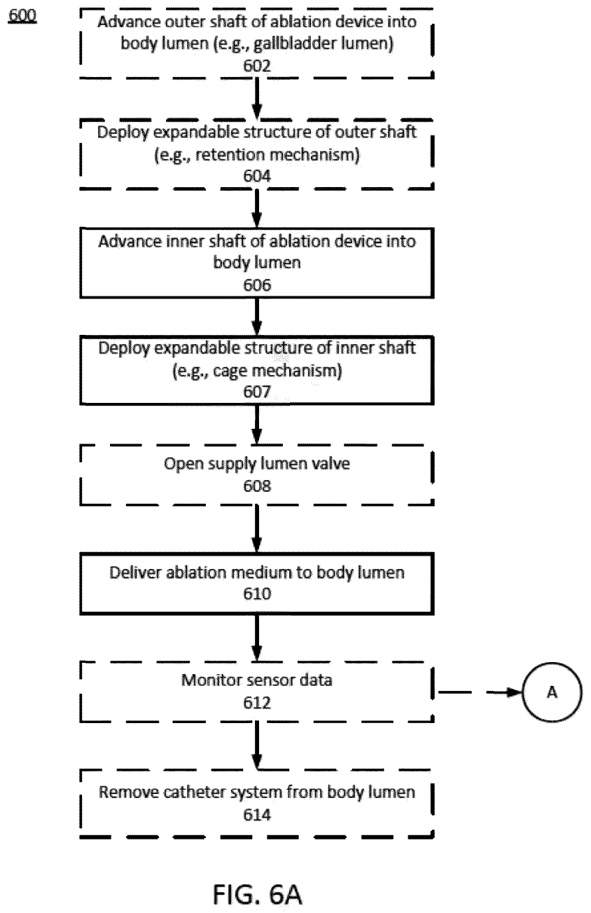

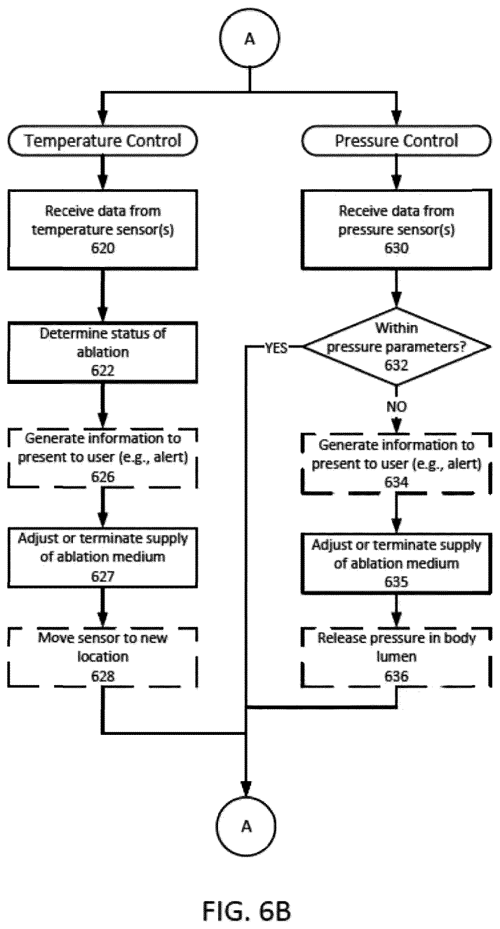

In some embodiments, the outer shaft 260 can include a tapered portion or tapered end at the distal end of the outer shaft 260. In some embodiments, a dilator can be inserted into a lumen of the outer shaft 260 (e.g., lumen 262) to aid in insertion of the outer shaft 260 into the body lumen BL. The dilator can be positioned in the lumen such that a distal end of the dilator extends distally from the outer shaft 260. In such instances, the tapered end of the outer shaft 260 can form a smooth transition from the outer shaft 260 to an outer surface of the dilator to aid in insertion into the body lumen BL, rather than having a sudden step in the profile of the device during insertion into the body lumen BL. Further details with respect to using a dilator with the outer shaft 260 are provided with reference to FIGS. 6A-6B.

In some embodiments, the outer shaft 260 includes an expandable structure or body 266 that can be deployed within the body lumen BL, e.g., transitioned from an undeployed state or configuration to a deployed or expanded state or configuration. The expandable structure 266 can be configured to prevent dislodgement and/or create a seal between the outer shaft 260 and the body lumen BL. In use, the outer shaft 260 can be advanced, e.g., along a guidewire, until a distal end of the outer shaft 260 is positioned within the body lumen BL through an opening. The expandable structure 266 can then be deployed (e.g., expanded, inflated), as schematically shown in FIG. 2 by arrows 290. Once deployed (e.g., once in its deployed state), the expandable structure can have a diameter larger than an diameter of the opening through which the outer shaft 260 has been placed and therefore be configured to retain the outer shaft 260 within the body lumen BL. In some embodiments, the expandable structure 266 includes an inflatable balloon, a shape memory structure (e.g., a deployable nitinol structure), etc. In some embodiments, the expandable structure 266 in its deployed state can have an outer diameter that is about 1.5 times to about 3 times larger than an outer diameter of the outer shaft 260.

In some embodiments, the expandable structure 266 can transition from an undeployed state to a deployed state via compression of a portion of the outer shaft 260 and/or movement of an inner shaft relative to an outer shaft. For example, the expandable structure 266 can be bounded within a region along the length of the outer shaft 260 between two boundary rings, and the expandable structure 266 can deploy (e.g., expand) upon bringing the two boundary rings closer together. In some embodiments, the outer shaft 260 can be formed of or include multiple concentric tubes or tubular members, e.g., an inner tubular member can be translated relative to an outer tubular member to move the ends of the expandable structure 266 closer to one another to expand the expandable structure 266 (e.g., to deploy the expandable structure 266). In such embodiments, at least one end of the expandable structure 266 (e.g., a proximal end) can be coupled to an outer tubular member and the other end of the expandable structure 266 (e.g. distal end) can be coupled to an inner tubular member, and translation of the inner tubular member relative to the outer tubular member can cause expansion or deployment of the expandable structure 266. In some embodiments, the expandable structure 266 can be pre-shaped to expand into its deployed state. For example, the expandable structure 266 can be held in tension (e.g., held in its undeployed state by an outer sleeve or tubular member, or stretched flat along an outer surface of the outer shaft 260 by a tubular member or pull wire), and when released, can self-expand into its deployed state.

In some embodiments, the expandable structure 266 can include elongate members (e.g., bands, fibers, wires, splines) arranged in a woven or braided pattern. In some embodiments, the elongate members can be bent to form a bulb-like shape upon transitioning of the expandable structure 266 from an undeployed state to a deployed state. In some embodiments, linear compression of one end of the elongate members relative to the opposite end of the elongate members, can expand the expandable structure 266 outward, creating a geometry with an expanded diameter. In some embodiments, the expandable structure 266 can have a larger diameter in the deployed state in comparison to the undeployed state. This expansion can aid in inhibiting unintentional removal of the expandable structure 266 from the body lumen BL. In some embodiments, the expandable structure 266 can be composed of nitinol, stainless steel, a polymer, or any suitable material that has a high strain relief. In some embodiments, the expandable structure 266 can be formed of shape-memory material, such as, for example, shape memory Nitinol.

In some embodiments, the expandable structure 266 can function as a seal that seals an opening through which the ablation catheter 250 is disposed. Further details of suitable expandable structures 266 implemented as a seal are described in International Patent Application No. PCT/US2019/017112, incorporated herein by reference.

While two lumens (e.g., lumens 262, 264) are depicted in FIG. 2, it can be appreciated that the outer shaft 260 can include any number of lumens, including a single lumen and/or more than two lumens. The outer shaft 260 can also include additional sensors, expandable structures, etc. according to embodiments described herein.

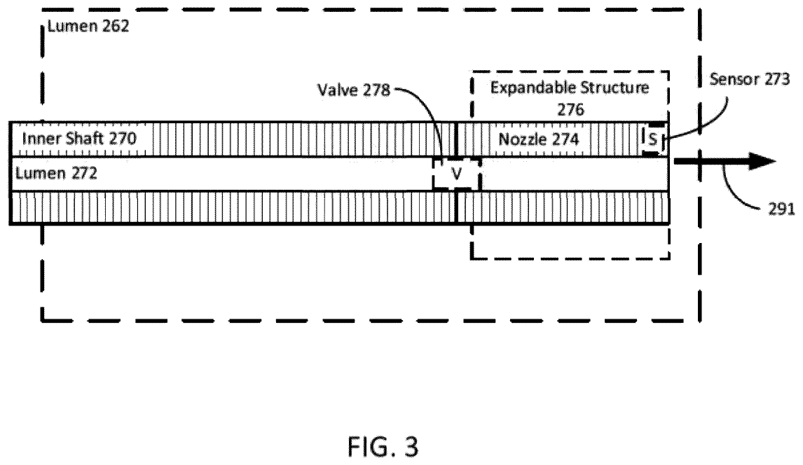

FIG. 3 provides a more detailed view of the inner shaft 270 disposed in the lumen 262. The inner shaft 270 can be deployed from the outer shaft 260 and the lumen 262 via movement in an axial direction (e.g., translation along a longitudinal axis of the outer shaft 260), as depicted by arrow 291. The inner shaft 270 can be, for example, an ablation delivery device or ablation catheter. In some embodiments, the inner shaft 270 can form a portion of a cryoablation device and be configured to deliver a cryogenic ablation medium into the body lumen BL. The inner shaft 270 can be configured to provide an ablative energy or an ablative medium capable of killing cells within the body lumen BL. For example, the inner shaft 270 can be configured to provide an ablative energy or ablative medium capable of killing cells in a mucosal layer of a gallbladder lumen, killing cells lining a cystic duct, or any combination thereof. The ablative energy or medium can include, for example, a chemical agent (e.g., an antibiotic, a liquid sclerosant, sodium tetradecyl sulphate, acetic acid, ethanol, hypertonic sodium chloride, urea), a cryogenic ablation medium (e.g., a cryogenic liquid or gas), thermal ablation, electrical ablation, etc. In some embodiments, the inner shaft 270 can be configured to deliver multiple types of ablative energies or mediums. The inner shaft 270 can be configured to provide ablation that is spatially diffuse. Stated differently, the inner shaft 270 can be configured to provide ablation that ablates a large area of a body lumen BL. In some embodiments, the inner shaft 270 can be configured to deliver ablation for defunctionalizing gallbladder mucosa, for ablating or sclerosis of a cystic duct, or any combination thereof.

In some embodiments, the inner shaft 270 can be configured to deliver thermal ablation, cryoablation, chemical ablation, or any combination thereof. In some embodiments, cryoablation involves delivering a low temperature fluid to wall of the gallbladder, such as liquid nitrogen. In some embodiments, cryoablation involves delivering an ablation medium to the gallbladder wall that induces low temperatures due to phase change, such as nitrous oxide or carbon dioxide. In some embodiments, thermal ablation involves delivering a high temperature fluid to the wall of the gallbladder, such as, for example, hot water or steam. In some embodiments, the ablative medium is delivered in a liquid form, a gaseous form, an aerosol form, a gel form, or any combination thereof.

The inner shaft 270 can define a lumen 272. The lumen 272 can be configured to deliver an ablation medium, e.g., from ablation medium supply 120, to a nozzle 274 that is disposable within the body lumen BL. The nozzle 274 can be configured to release the ablation medium into the body lumen BL. In some embodiments, the nozzle 274 can include a plurality of openings or fenestrations for distributing the ablation medium throughout the body lumen BL. In some embodiments, the lumen 272 and nozzle 274 can be configured to convey a cryogenic ablation medium in a liquid state into the body lumen BL. The lumen 272 and nozzle 274 can be configured with dimensions that maintain a set amount of pressure on the cryogenic ablation medium such that the medium does not undergo a liquid-to-gas transition until the ablation medium exits the openings of the nozzle 274. Stated differently, the lumen 272 and nozzle 274 can be configured to convey a cryogenic ablation medium in a liquid state to the openings of the nozzle 274, at which point the release of the cryogenic ablation medium into the body lumen BL results in the cryogenic ablation medium changing from the liquid state into a gas state. In some embodiments, the lumen 272 of the inner shaft 270 can have a diameter from about 0.001 inches to about 0.1 inches, including all values and subranges in between.

In some embodiments, the inner shaft 270 can include an expandable structure or body 276. In some embodiments, the expandable structure can be disposed about the nozzle 274. In some embodiments, the expandable structure 276 can expand within the body lumen BL, such that the nozzle 274 is centered within the body lumen BL. In other words, the expandable structure 276 can expand outward to a desired diameter, such that a radial distance from the center of the nozzle 274 to the walls of the body lumen BL is consistent or approximately consistent in all radial directions. This consistent spacing or centering can ensure a minimum radial distance between the nozzle 274 and nearby tissue of the body lumen BL and/or more even distribution of the ablation medium through the body lumen BL. This can allow for ablation of luminal tissue, while ensuring that the ablation medium is not too close of a range (e.g., creating a sticking or perforation risk) or too far of a range (e.g., reducing the effectiveness of the ablation) from a section of tissue.

In some embodiments, the profile and/or the thermal mass of the expandable structure 276 can be minimized to allow for more efficient passage of ablation medium from the nozzle 274 to the surfaces of the body lumen BL. In other words, reducing or minimizing both the physical size and the amount of heat energy the expandable structure 276 can absorb or radiate can improve the efficiency of heat transfer during ablation. In some embodiments, the expandable structure 276 can be composed of Nitinol, stainless steel, a polymer, or any suitable material that has a high strain relief. In some embodiments, the material of the expandable structure 276 can be selected based on the material's ability to withstand cryogenic temperatures without significantly altering the cooling performance of the catheter system 250. In some embodiments, the use of an expandable structure 276 can avoid the creation of a significant apposition force between the expandable structure 276 and the body lumen BL, contrary to cryoablation balloon catheters. This can create a more effective cooling method that is less sensitive to the contents and geometry of the body lumen BL.

In some embodiments, the expandable structure 276 can be collapsible or retractable, such that the inner shaft 270 can be removed from the body lumen BL. In some embodiments, the expandable structure 276 can be radially symmetrical in order to ensure equidistant or approximately equidistant radial spacing of the walls of the body lumen BL around the outside of the nozzle 274.

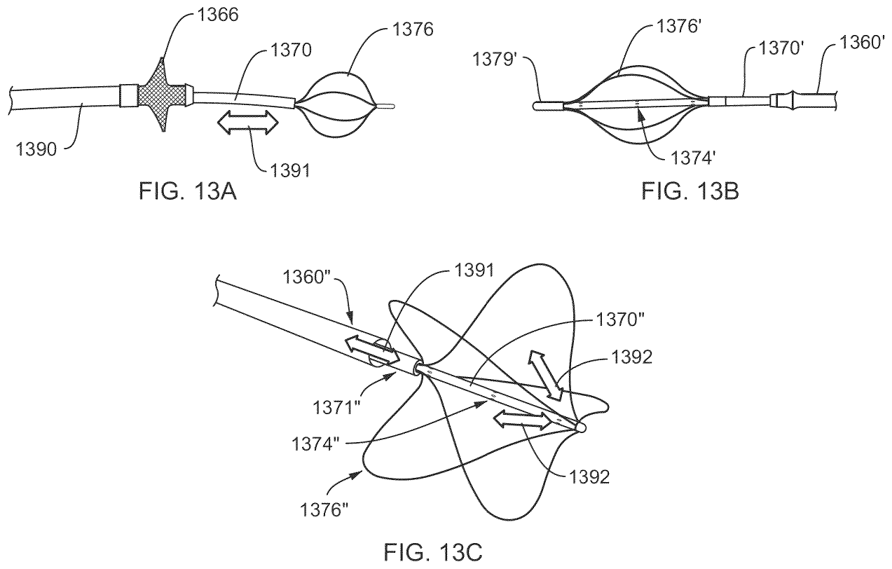

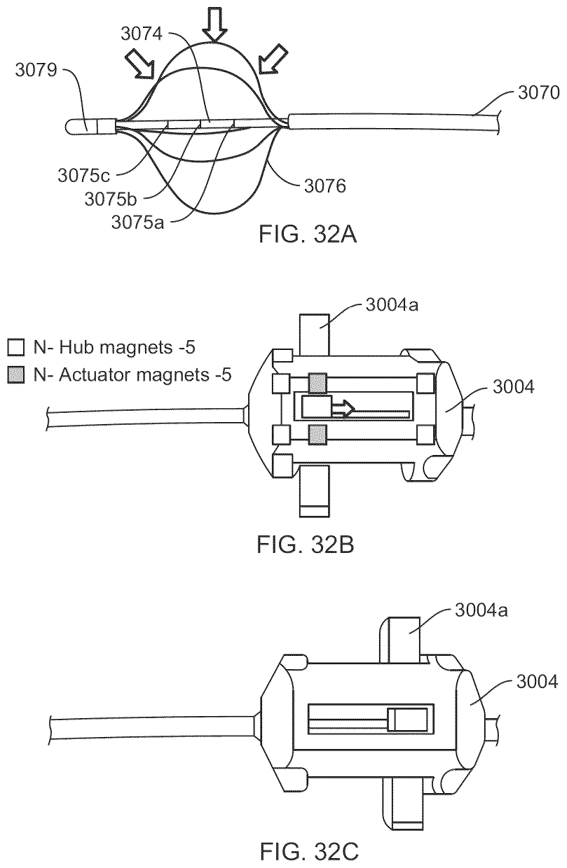

In some embodiments, the expandable structure 276 can transition from an unexpanded state (e.g., undeployed state) to an expanded state (e.g., deployed state) via compression of a portion of the inner shaft 270 and/or movement of one portion of the inner shaft 270 relative to another portion of the inner shaft 270. In some embodiments, the expandable structure 276 can include elongate members (e.g., bands, wires, fibers, splines) arranged in a woven or braided pattern or arranged individually along a length of the inner shaft 270. For example, the expandable structure 276 can include one or more elongate members that generally extend along a length of the inner shaft 270. In some embodiments, the expandable structure 276 can include a single expandable elongate member, while in other embodiments, the expandable structure can include between 2 or 20 elongate members, including all values and subranges in-between. In some embodiments, a distal end of the expandable structure 276 can move toward a more proximal point of the expandable structure 276, causing the expandable structure 276 to expand, i.e., to transition from an undeployed state or configuration to a deployed state or configuration. In some embodiments, the inner shaft 270 can move relative to a sleeve or tubular member (not shown) to cause the expandable structure 276 to expand and contract. For example, the sleeve can be used to hold the expandable structure 276 in an undeployed state or the sleeve can move one end of the expandable structure 276 (e.g., a proximal end) relative to the other end of the expandable structure 276 (e.g., a distal end) to expand the expandable structure 276 into its expanded state. In some embodiments, the expandable structure 276 can include a plurality of wires or bands that extend along the length of the inner shaft 270, such that the wires or bands can be advanced and retracted from a proximal end of the ablation catheter 250. Such advancement and retraction can be used to deploy and undeploy the expandable structure 276. Further details of mechanism of expandable structures 276 are described with reference to later figures, including, for example, FIGS. 13A-13C and FIGS. 32A-32C.

In some embodiments, the inner shaft 270 can optionally include a valve 278. The valve 278 can be configured to control delivery of the ablation medium into the body lumen BL. For example, the valve 278 can be configured to turn on or shut off supply of the ablation medium into the nozzle 274. In some embodiments, a control unit (e.g., control unit 110) can be configured to control opening and/or closing of the valve 278. In some embodiments, a mechanical actuator (e.g., coupled to a handheld device, as described above) can be used to open and/or close the valve 278. In some embodiments, the valve 278 can be configured to close (e.g., automatically and/or via control by a control unit) in response to a pressure within the body lumen BL being greater than a predetermined threshold. In some embodiments, a sensor (e.g., sensor disposed on inner or outer shaft 260, 270 and/or sensor coupled to control unit 110) can be used to measure the pressure within the body lumen BL and control the valve 278 to open and/or close. In some embodiments, the valve 278 can be configured to close in response to a pressure difference between the body lumen BL and an evacuation lumen (e.g., lumen 262), e.g., indicating that a blockage or obstruction has formed along an evacuation pathway. For example, multiple sensors can be configured to measure different pressures associated with the catheter system 250 and/or body lumen BL, and a control unit (e.g., control unit 110) can be configured to analyze when such pressure measurements to determine when an unexpected obstruction has isolated any fluid flow paths into and/or out of the body lumen BL.

As noted above, in some embodiments, the inner shaft 270 can be or form part of a cryoablation device and be configured to deliver a cryogenic ablation medium into the body lumen BL. The cryoablation device can leverage the phase-change properties of certain cryogenic ablation mediums (e.g., liquid nitrous oxide) to induce cryoablation temperatures at a target tissue interface. When such cryogenic ablation mediums transition from liquid to gas, they expand in volume and can cause increase in pressure within the body lumen BL. Therefore, one important consideration in designing systems and devices disclosed herein lies in the monitoring and control of the intraluminal pressure in the body lumen BL during an ablation procedure. For example, systems and devices disclosed herein can be configured to ensure that intraluminal pressure does not increase above a predetermined threshold and/or lies within a predetermined range. In instances where there is an increase in intraluminal pressure (e.g., pressure above a predetermined threshold, or sudden change in pressure above a predetermined rate), systems and devices disclosed herein can be configured to evacuate air, gaseous cryoablation medium, and/or other fluids from within the body lumen BL to reduce the intraluminal pressure. In such instances, it can be important to ensure to any cryogenic ablation medium within the catheter system 250 (e.g., within lumen 272 of the inner shaft 270) and/or supply line into the catheter system 250 does not exit the catheter system 250 (e.g., nozzle 274) into the body lumen BL, further adding to the pressure increase. Accordingly, it can be desirable to minimize or reduce the amount of residual cryogenic ablation medium that is delivered into the body lumen BL in response to detecting a pressure increase event (e.g., pressure above a predetermined threshold, or sudden change in pressure above a predetermined rate). In some embodiments, the valve 278 can be used to reduce the amount of residual cryogenic ablation medium that is delivered into the body lumen 270. The valve 278 can be positioned at or near the nozzle 274 such that the valve 278, upon closing, prevents any residual or excess ablation medium within the lumen 272 and/or other passageways leading to the nozzle 274 from being delivered into the body lumen BL.

The valve 278 can include any range of suitable mechanisms. In some embodiments, the valve can be closed in its resting state but can open to allow ablation medium to be delivered into the body lumen BL. Alternatively, the valve 278 can be open in its resting state and can be closed to prevent additional ablation medium from being delivered into the body lumen BL. In some embodiments, the valve 278 can be biased closed and/or open using a spring mechanism. The valve 278 can have any suitable geometry including, for example, a cube, cone, cylinder, triangular prism, torus, helix, ovoid, or other three-dimensional body with sufficient structure to impede ablation medium flow. In some embodiments, the valve 278 can be seated against a valve seat defined within the inner shaft 270 (e.g., within lumen 272). In some embodiments, the valve 278 can be actuated, either manually or via a control device (e.g., control device 110), with a drive wire or rod, pneumatic or hydraulic pressure, electromagnetic force, and/or motor to open and/or close. Examples of suitable valves are described in International Patent Application No. PCT/US2020/045436, incorporated herein by reference.

In some embodiments, the inner shaft 270 optionally includes a sensor 273. In some embodiments, the sensor 273 can be located in a distal portion of the inner shaft 270 that is configured to be disposed within the body lumen BL. Alternatively, the sensor 273 can be disposed at a different location along the inner shaft 270, including, for example, within a lumen (e.g., lumen 272), at a proximal end at the inner shaft 270, etc. The sensor 273 can be configured to capture information about an environment within the body lumen BL. For example, the sensor 273 can be configured to measure a property (e.g., pressure, temperature) of an ablation medium being delivered to the body lumen BL, a property (e.g., pressure, temperature) of the body lumen BL or fluid within the body lumen BL, etc. The sensor 273 can include, for example, a pressure sensor (e.g., pressure transducer, strain gauge transducer, diaphragm displacement sensor, optical fiber pressure sensor, solid state sensor), temperature sensor, light sensors, gas sensors, etc. Sensor 273 can be capable of communicating data (e.g., sensor measurements) to a control unit (e.g., control unit 110) and/or other compute device (e.g., compute device 190) via a wired or wireless connection.

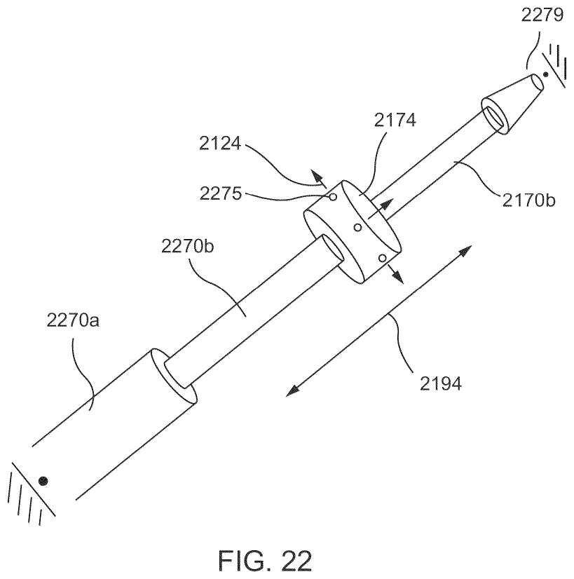

In some embodiments, the inner shaft 270 can optionally include one or more additional lumens. In some embodiments, a lumen can configured as a passageway for relaying pressure information or other conditions (e.g., temperature) from the body lumen BL and/or other portions of the catheter system 250. In some embodiments, the catheter system 250 can optionally include an occluder, as further described with reference to FIG. 22. While a single lumen (e.g., lumen 272) is depicted in FIG. 3, it can be appreciated that the inner shaft 270 can include any number of lumens, including a single lumen and/or more than two lumens. The inner shaft 270 can also include additional sensors, valves, nozzles, etc. according to embodiments described herein.

FIG. 4 provides a detailed view of the inner shaft 270 and the outer shaft 260 positioned within the body lumen BL. The inner shaft 270 can be disposed within the lumen 262 of the outer shaft 260. The spacing between an outer surface of the inner shaft 270 and an inner surface of the lumen 262 can define an evacuation lumen or passageway for removing gas and/or other fluids from the body lumen BL (e.g., ablation medium from the body lumen BL).

The outer shaft 260 and/or the inner shaft 270 can be formed of flexible and/or semi-flexible material that enables each to be navigated to the body lumen BL, e.g., along a guidewire. The material can be a medical grade, biocompatible material. The inner shaft 270 can be deployed into the body lumen BL in an axial direction depicted by arrow 294. The expandable structure 266 of the outer shaft 260 and the expandable structure 276 of the inner shaft 270 can be deployed radially, as depicted by arrows 292.

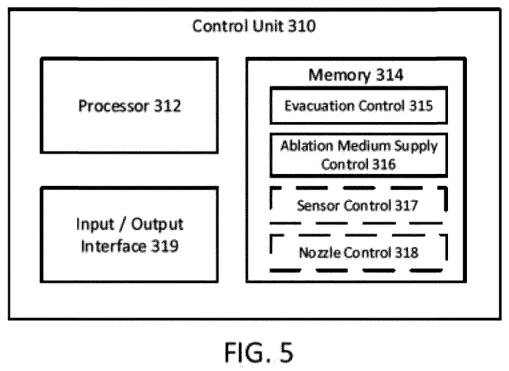

FIG. 5 is a schematic illustration of an example control unit 310, according to some embodiments. Control unit 310 can be structurally and/or functionally similar to control unit 110, as described with reference to FIG. 1. For example, control unit 310 can be configured to control one or more components of an ablation system and/or catheter system (e.g., ablation system 100, catheter system 250). Control unit 310 can include a processor 312, a memory 314, and an input/output interface 319. In some embodiments, the control unit 310 can be coupled to the catheter system, e.g., by being contained in a handheld device that is coupled to a proximal end of the catheter system. In some embodiments, the control unit 310 can be remotely situated, e.g., on a remote compute device or system, and can be used to remotely control the operation of the catheter system.

Processor 312 of control unit 310 can be any suitable processing device configured to run and/or execute functions associated with deploying one or more components of a catheter system (e.g., advancing or retracting a shaft, deploying an expandable structure, opening and/or closing a valve), delivering ablation medium into a body lumen, analyzing sensor data associated with an ablation procedure involving the catheter system, controlling temperature and/or pressure within the body lumen, etc. Processor 312 can be configured to execute modules, functions, and/or processes. Processor 312 can be a general purpose processor, a Field Programmable Gate Array (FPGA), an Application Specific Integrated Circuit (ASIC), a Digital Signal Processor (DSP), and/or the like. In some embodiments, processor 312 is part of a circuit, e.g., such as an integrated circuit. In some embodiments, one or more other components of the ablation system can be integrated into the circuit, including, for example, one or more sensors.

Input/output interface 319 can include a user interface and/or communication interfaces for connecting the control unit 310 to one or more external compute devices. The user interface(s) can include one or more components that are configured to receive inputs and send outputs to other devices and/or a user operating a device, e.g., a user operating a catheter system. For example, the user interface can include a display device (e.g., a display, a touch screen, etc.), an audio device (e.g., a speaker or alarm), and one or more additional input/output device(s) configured for receiving an input and/or generating an output to a user. The communication interface(s) can include one or more wireless and/or wired interfaces, e.g., for communicating with other compute device (e.g., compute device(s) 190) via one or more networks (e.g., a local area network (LAN), a wide area network (WAN), a virtual network, a telecommunications network).

Memory 314 can be, for example, a random access memory (RAM), a memory buffer, a hard drive, a database, an erasable programmable read-only memory (EPROM), an electrically erasable read-only memory (EEPROM), a read-only memory (ROM), and/or so forth. In some embodiments, memory 314 stores instructions that cause processor 312 to execute modules, processes, and/or functions associated with deploying one or more components of a catheter system (e.g., advancing or retracting a shaft, deploying an expandable structure, opening and/or closing a valve), delivering ablation medium into a body lumen, analyzing sensor data associated with an ablation procedure involving the catheter system, controlling temperature and/or pressure within the body lumen, etc. Methods as described herein can be implemented by way of machine (e.g., computer processor) executable code stored on an electronic storage location of the control unit 310, such as, for example, on the memory 314, or a memory operatively coupled to the control unit 310. In some embodiments, the machine executable or machine-readable code is provided in the form of software. In operation, the code can be executed by the processor 312. In some cases, the code is retrieved from the memory 314 to be accessed and/or executed by the processor 312.

As depicted in FIG. 5, memory 314 stores instructions that can cause processor 312 to execute modules, processes, and/or functions, illustrated as evacuation control 315, ablation medium supply control 316, optionally sensor control 317, and/or optionally nozzle control 318. Evacuation control 315, ablation medium supply control 316, sensor control 317, and/or nozzle control 318 can be implemented as one or more programs and/or applications that are tied to hardware components. For example, evacuation control 315, ablation medium supply control 316, sensor control 317, and/or nozzle control 318 can be implemented by one or more components of an ablation system and/or catheter system (e.g., ablation system 100, catheter system 250). In some embodiments, the processor 312 executing evacuation control 315 can control the opening of a valve and/or activation of a vacuum source to evacuate gas or other fluid (e.g., ablation medium) from a body lumen. In some embodiments, the processor 312 executing ablation medium supply control 316 can control the opening of a valve and/or operation of an ablation supply source to deliver an ablation medium into a body lumen via a catheter system. In some embodiments, the processor 312 executing sensor control 317 can receive, process, and/or analyze data from one or more sensors and/or use such data to control the operation of one or more other components of the ablation system or catheter system.

In some embodiments, the nozzle control 318 can be implemented to control positioning or movement of one or more nozzles (e.g., nozzle 274) within a body lumen (e.g., body lumen BL). In some embodiments, the nozzle control 318 can be implemented to rotate an ablation catheter (e.g., inner shaft 270) along its central axis to increase uniform or more distributed delivery of a liquid cryogen medium. In some embodiments, the nozzle control 318 can actuate the ablation catheter and/or the nozzle to move axially or linearly to increase distribution of cryogen from the nozzle. In some embodiments, the nozzle control 318 can be implemented to open and close one or more nozzle openings.

FIGS. 6A-6B depict an example method 600 for ablation and managing pressure and/or temperature during an ablation procedure, according to an embodiment. In some embodiments, the ablation procedure can be a cryoablation procedure that is implemented using an ablation system (e.g., ablation system 100) that includes a cryoablation device. In some embodiments, the ablation procedure is performed in the gallbladder to defunctionalize the gallbladder. The method 600 optionally includes advancing an outer shaft (e.g., outer shaft 260) of a catheter system (e.g., catheter system 150, 250) of the ablation system into a body lumen (e.g., gallbladder lumen), at 602. In some embodiments, ultrasonic imaging can be used to visualize the body lumen. In some embodiments, a standard needle and guidewire can be used to access the body lumen via a trans-hepatic approach, e.g., using the Seldinger technique. When the target body lumen is the gallbladder, visual return of bile through the needle and fluoroscopic confirmation of the guidewire curling inside the body lumen can assist with validating proper placement of a guidewire within the body lumen. In some embodiments, the outer shaft can be advanced along the guidewire with a dilator positioned within a lumen (e.g., lumen 262) of the outer shaft. In some embodiments, a series of progressively larger dilators can be advanced along the guidewire to dilate the tract into the body lumen. After dilating the tract, the outer shaft (e.g., with a dilator inserted within) can be advanced along the guidewire into the body lumen. In some embodiments, the outer shaft of the catheter system can be a separate tubular structure, e.g., an access sheath or an introducer, that is first placed within the body lumen before placing an inner shaft within the body lumen. Alternatively, in some embodiments, the outer and inner shafts can be placed into the body lumen simultaneously. In some embodiments, placement of the outer shaft can be similar to a percutaneous drainage tube placement technique. Once the outer shaft has been positioned in the body lumen, the dilator and guidewire can be removed to enable placement of an inner shaft within the outer shaft, as further described below.

In some embodiments, the catheter system can be placed into a gallbladder lumen. Accessing the gallbladder with the catheter system can be achieved through a percutaneous approach. In some embodiments, an access sheath or outer shaft 760 of the catheter system accesses the gallbladder 2 through a transhepatic, percutaneous approach using ultrasound guidance, as seen in FIG. 7A. In some embodiments, the access sheath 760 of the catheter device accesses the gallbladder 2 through a subhepatic, percutaneous approach using ultrasound guidance, as seen in FIG. 7B. In some embodiments, the percutaneous approach is similar to the method used to place a cholecystostomy drain. In some embodiments, the access sheath 760 provided herein accesses the gallbladder 2 endoscopically, as shown in FIG. 7C. In some embodiments, the access sheath 760 accesses the gallbladder 2 utilizing native anatomy by creating a transmural stoma connecting the inner lumen of the gallbladder to the lumen of the small bowel, as shown in FIG. 7C. In some embodiments, percutaneous access is gained using a hollow bore needle, whereby a guidewire is placed through the needle to create a tract to the desired access location (e.g., a cystic duct, a gallbladder, or a combination thereof). In some embodiments, the access sheath 760 and an inner shaft or ablation catheter are configured with a concentric lumen to enable a guidewire to pass through. In some embodiments, the access sheath 760 and the ablation catheter are configured with a non-concentric lumen to enable a guidewire to pass through.

After positioning the distal end of the outer shaft of the catheter system within the body lumen, the method 600 can optionally include deploying an expandable structure (e.g., expandable structure 266) of the outer shaft, at 604. Deploying the expandable structure within the body lumen can ensure that the outer shaft (e.g., access catheter, introducer) remains or is retained within the body lumen during the ablation procedure. In some embodiments, deploying the expandable structure can involve moving a first tubular member relative to a second tubular member to bring a first end of the expandable structure toward the second end of the expandable structure, thereby causing the expandable structure to expand outwards. In some embodiments, expanding the expandable structure can involve releasing tension placed on the expandable member (e.g., by releasing a sheath or pull wire) and allowing the expandable structure to automatically expand or self-expand into a pre-formed shape.

The method 600 can include advancing an inner shaft (e.g., inner shaft 270) of the catheter system into the body lumen, at 606. In some embodiments, where a dilator was positioned in the outer shaft to advance the outer shaft into the body lumen, the inner shaft can be advanced after removal of the dilator. The inner shaft can be advanced until a nozzle (e.g., nozzle 274) of the inner shaft is disposed within the body lumen distal to a distal end of the outer shaft. The inner shaft can be advanced into the body lumen by inserting the inner shaft into a lumen defined by the outer shaft and advancing the inner shaft through that lumen until a distal portion of the inner shaft is disposed distal to the outer shaft. The distal portion of the inner shaft can include one or more openings (e.g., fenestrations) that can deliver ablation medium into the body lumen. In some embodiments, the method 600 can optionally include deployment of saline to lavage and drain any content within the body lumen, e.g., via inner and/or outer shafts. For example, fluid such as saline can be delivered into the gallbladder via a first lumen (e.g., lumen 272 defined by inner shaft 270) and/or content within the body lumen (e.g., gallbladder content) can be evacuated from the body lumen via a second lumen (e.g., lumen 262 defined by outer shaft 260).

The method 600 includes deployment of an expandable structure (e.g., expandable structure 276) of the inner shaft, at 607. In some embodiments, the expandable structure can include a plurality of wires or bands that extend along a length of the inner shaft. Such wires can be deployed by advancing the wires distally out of a sheath. In some embodiments, the expandable structure can be deployed by moving inner and outer tubular members relative to one another. Once the expandable structure is deployed, the expandable structure can center the nozzle within the body lumen or ensure that the nozzle is at least a predetermined distance away from a tissue surface. The method 600 can optionally include opening a supply lumen valve (e.g., valve 278), at 608. For example, as discussed above with reference to FIG. 3, a valve can be positioned along an ablation medium delivery passageway (e.g., along lumen 272 defined by inner shaft) to control delivery of the ablation medium. The valve in its open state can allow ablation medium to flow past the valve and into the body lumen, while the valve in its closed state can block the flow of ablation medium into the body lumen. In some embodiments, the valve can naturally be in a closed state, and therefore method 600 can include opening the valve such that ablation medium can be delivered into the body lumen. In some embodiments, the valve can naturally be in an open state, and therefore 608 can be omitted.

The method 600 can include delivering the ablation medium to the body lumen, at 610. In some embodiments, a cartridge (e.g., ablation medium supply source 120, 220) of a cryogenic ablation medium (e.g., nitrous oxide) or any other suitable ablation medium can be loaded into a handle (e.g., handheld device) of the ablation device. In the case of using a cryogenic ablation medium, 1, 2, 3, 4, 5, 6, 7, 8, 9, 10 or more than 10 freeze-thaw cycles can be delivered to the gallbladder, at 610, to ensure complete hypothermic death of the gallbladder mucosa. While delivering the ablation medium, method 600 can include events and/or steps associated with pressure and/or temperature monitoring at 612, as further described below with reference to FIG. 6B. For example, a control unit (e.g., control unit 110, 310) can monitor pressure, temperature, and/or other conditions to ensure safe delivery of the ablation medium. After delivering the ablation medium, the method 600 can optionally include removing the catheter system (e.g., inner and outer shafts) from the body lumen, at 614.

FIG. 6B illustrates events and steps associated with a temperature and pressure monitoring protocol performed during an ablation procedure, such as the ablation procedure described with reference to FIG. 6A. The control unit (e.g., control unit 110, 310) of the ablation system or another compute device can be configured to receive data from a temperature sensor, at 620, and determine a status of ablation, at 622. In some embodiments, the status of the ablation can be determined by data received from the temperature sensor. In some embodiments, the temperature sensor can be integrated into one of the inner or outer shafts (e.g., as sensor 263, 273). In other embodiments, the temperature sensor can be operatively coupled to a lumen that extends into the body lumen, which the sensor can use to measure a temperature associated with the body lumen. In still other embodiments, the temperature sensor can be mounted to a probe that is separately insertable into the body lumen, e.g., via a separate lumen (e.g., lumen 264) and/or the same lumen that houses the inner shaft (e.g., lumen 262). In some embodiments, the temperature sensor can be implanted or inserted into tissue within the body lumen such that a temperature of the tissue can be measured.

The method 600 can optionally include generating information to present to a user, at 626. In some embodiments, the information presented to the user can be presented via a compute device, such as control unit 110 and/or 310, or other compute device in network communication with the ablation system (e.g., a tablet, smartphone, or any other suitable communication device). Based on the status of the ablation determined at 622, the supply of ablation medium can be adjusted or terminated at 627. In some embodiments, the supply of ablation medium can be reduced or terminated. In some embodiments, the supply of ablation medium can be increased. In some embodiments, to evaluate the temperature of tissue at a different location, the temperature sensor can optionally be moved to a new location, at 628. For example, the temperature sensor can be retracted from tissue a first location, moved to a second location, and inserted into tissue at a second location. In some embodiments, the method 600 can include determining whether the ablation is completed, e.g., based on sensor data, and in response to determining that the ablation has completed, the supply of ablation medium can be terminated (e.g., by closing valve 278), and the method 600 can continue to 612, where the catheter system is removed from the body lumen.

The control unit (e.g., control unit 110, 310) or another compute device can also receive pressure data from one or more pressure sensors, at 630. In some embodiments, a first pressure reading can be from inside the body lumen (e.g., measuring intraluminal pressure), while second pressure reading can be from inside the outer shaft (e.g., measuring pressure within the evacuation lumen (e.g., lumen 262)). In other embodiments, more or less pressure readings can be received at 630. At 632, at least one of the pressure measurements (e.g., intraluminal pressure within body lumen) is evaluated to determine if the pressure reading is within desired pressure parameters (e.g., within a desirable pressure range). If the pressure readings are substantially different from one another (e.g., the different pressure readings differ more than a predetermined amount or percentage from one another, or have a percentage (e.g., 30%) increase or decrease from a nominal operating pressure), or if one or more pressure readings are not within one or more desired pressure parameters (632: NO), information (e.g., an alert) can optionally be presented to the user, at 634, and the supply of ablation medium can be adjusted or terminated, at 635. The information presented to the user can indicate to the user that an error has occurred with the ablation delivery and/or operation of the device. For example, a substantial difference (e.g., difference above a predetermined amount or percentage) between an intraluminal pressure within the body lumen and a pressure within the evacuation lumen (such as the intraluminal pressure being greater than the evacuation lumen pressure) can indicate that a blockage has occurred at some point between the body lumen and the evacuation lumen. With cryogenic delivery systems, such can occur when ice or other solid content blocks a portion of an evacuation lumen. Such blockage can cause a pressure buildup in the body lumen and can result in injury to a patient. Therefore, in such cases, the control unit or other compute device can terminate supply of an ablation medium into the body lumen until the blockage is removed (e.g., via heating coils). In some embodiments, when a pressure measurement is outside of certain pressure parameters (e.g., a predetermined threshold value or range), the control unit can control one or more valves and/or a vacuum source (e.g., vacuum source 130) to evacuate ablation medium from the body lumen so as to reduce pressure buildup within the body lumen, at 636.

At 614, the catheter system (e.g., introducer and ablation catheter) can be removed from the body lumen. For a time period (e.g., a few weeks) after the removal of the ablation system, the body's chronic inflammatory response can scar the ablated gallbladder tissue, leading to involution of the lumen and occlusion of the cystic duct. Bile flow can be shut off to the gallbladder, while its blood supply remains uncompromised, resulting in an inert organ.

In some embodiments, as described above, the ablation procedures described herein use a cryogenic ablation medium. In some embodiments, the cryogenic ablation medium is a liquid. In some embodiments, the cryogenic ablation medium is a gas. In some embodiments, the cryogenic ablation medium undergoes a liquid-to-gas phase transition when being delivered using the catheter devices and nozzles disclosed herein. In some embodiments, cryoablation is achieved via the refrigerant property due to the liquid to gas phase change from an ablation medium, such as liquid nitrous oxide, carbon dioxide, and argon. In some embodiments, the phase change of the cryogenic ablation medium is triggered by a sudden reduction in pressure. In some embodiments, the phase change of the cryogenic ablation medium occurs when the liquid ablation medium contacts a wall of the body lumen (e.g., wall of the gallbladder). As such, the liquid ablation medium can be delivered into the body lumen and contact the wall of the body lumen and phase change into a liquid ablation medium. Ablation can happen at the phase change interface.

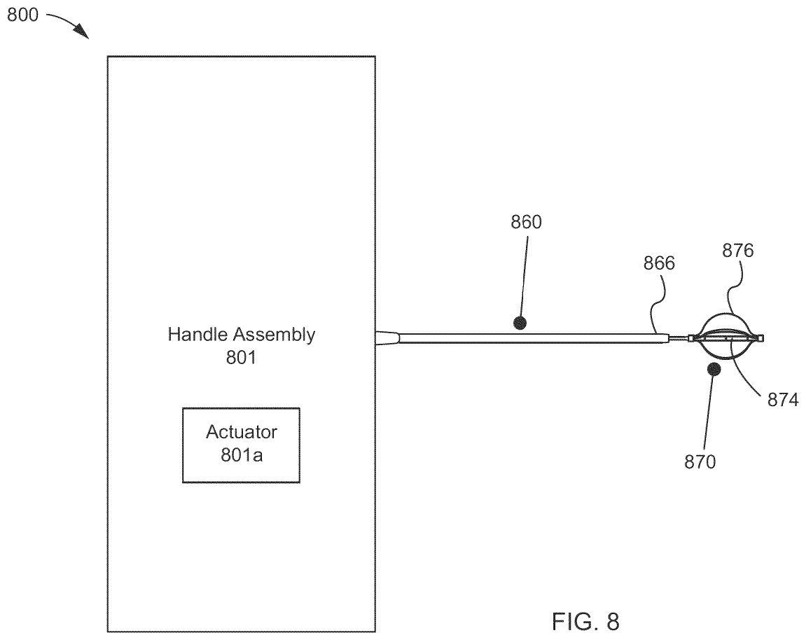

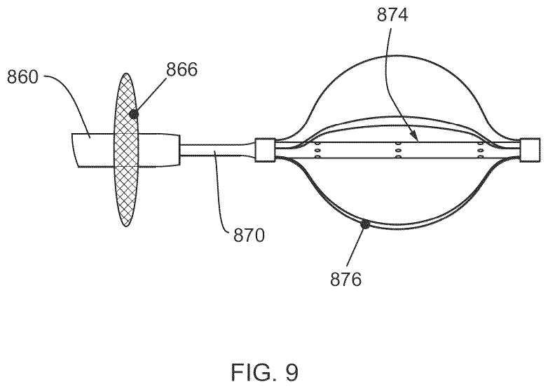

FIGS. 8-9 illustrate an ablation system implemented as a cryoablation device 800, according to an embodiment. The cryoablation device 800 can be configured to ablate or defunctionalize a gallbladder cavity. The cryoablation device 800 can include components that are structurally and/or functionally similar to other ablation systems and components thereof described herein (e.g., ablation system 100, catheter system 250, control unit 310, etc.) As shown the cryoablation device 800 includes a handle assembly 801, an outer shaft 860, and an inner shaft 870. The outer shaft 860 includes an expandable structure 866. The inner shaft 870 includes a nozzle 874 and an expandable structure 876. In some embodiments, the handle assembly 801 can include or house a control unit (e.g., control unit 110). In some embodiments, the handle assembly 801 can include an actuator 801a (e.g., a button) or multiple actuators 801a. In some embodiments, actuators can be used to control deployment of the expandable structure 866 on the outer shaft 860, the expandable structure 876 on the inner shaft 870, deployment of ablation medium through the nozzle 874, actuation of nozzle 874 (e.g., translation of rotation of nozzle 874), etc. In some embodiments, the handle assembly 801 can be fluidically coupled to an ablation medium supply (e.g., ablation medium supply 120). In some embodiments, the handle assembly 801 can include a user interface (e.g., input/output interface 319) to communicate information to the user and/or receive inputs from the user.

FIG. 9 is a detailed view of the outer shaft 860 and the inner shaft 870 of the cryoablation device 800, according to an embodiment. The outer shaft 860 and the inner shaft 870 can include components that are structurally and/or functionally similar to other ablation systems and components thereof described herein (e.g., outer shaft 260 and the inner shaft 270, etc.). As shown, the expandable structure 876 can be implemented as an expandable cage mechanism. As shown, the expandable structure 876 has a “closed” design, e.g., the bands or wires that form the expandable structure 876 come together and are closed on both the proximal end and the distal end of the expandable structure 876 such that the expandable structure 876 forms an enclosed basket or cage.

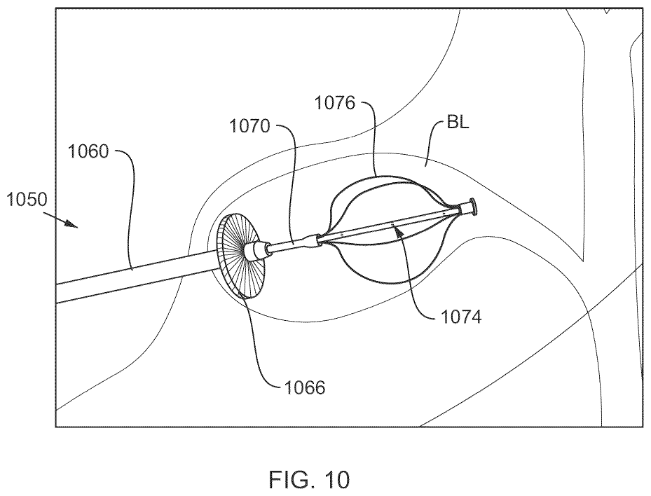

FIG. 10 shows an ablation catheter 1050 deployed in a body lumen BL, according to an embodiment. The ablation catheter 1050 can include components that are structurally and/or functionally similar to other ablation systems and components thereof described herein (e.g., ablation system 100, catheter system 250, control unit 310, cryoablation device 800, etc.). As shown, the ablation catheter 1050 includes an outer shaft 1060 with an expandable structure 1066 and an inner shaft 1070 with a nozzle 1074 and an expandable structure 1076. As shown, the expandable structure 1066 on the outer shaft 1060 is in the deployed state, such that the ablation catheter 1050 is inhibited from unintentionally exiting the body lumen BL (e.g., is maintained in position within the body lumen BL). The expandable structure 1076 on the inner shaft 1070 is in the deployed state to approximately center the nozzle 1074 within the body lumen BL, e.g., to ensure uniform and/or minimum spacing (e.g., a predetermined amount of spacing) between the nozzle 1074 and a tissue wall of the body lumen BL. As described above, such placement of the nozzle 1074 increases effectiveness of the ablation delivery and reduces potential undesirable effects (e.g., injury, attachment between nozzle and tissue, etc.). The expandable structure 1066 and the expandable structure 1076 are both collapsible (e.g., transitionable back into an undeployed state), such that the ablation catheter 1050 can be retracted from the body lumen BL.

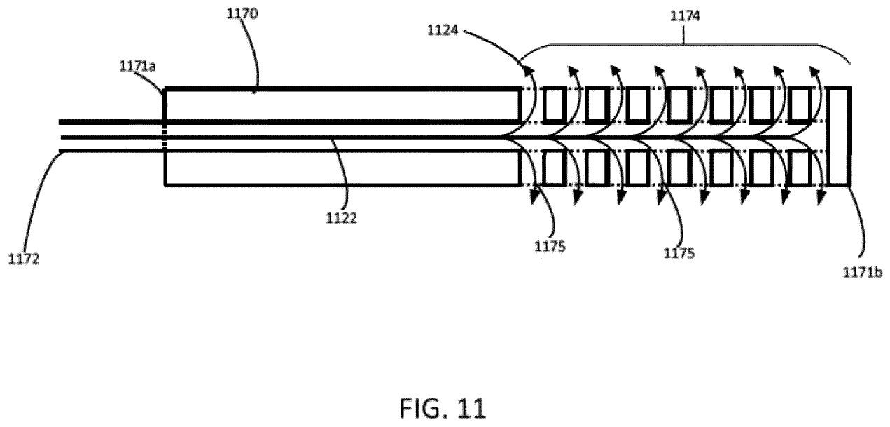

FIG. 11 illustrates an example of an inner shaft 1170 comprising a lumen 1172 and a fenestrated nozzle 1174. The inner shaft 1170 can include components that are structurally and/or functionally similar to other ablation systems and components thereof described herein (e.g., the inner shaft 270, inner shaft 870, etc.). In some embodiments, the inner shaft 1170 can include a proximal end 1171a and a distal end 1171b. In some embodiments, the lumen 1172 is sufficiently small to keep the cryogenic liquid ablation medium 1122 in a liquid state, with the cryogenic liquid ablation medium 1122 transitioning into a cryogenic gas ablation medium 1124 (i.e., a liquid-to-gas phase transition) as it exits the inner shaft 1170 via a plurality of fenestrations 1175, as shown in FIG. 11 (e.g., due to a pressure drop between inside of lumen 1172 and an inside of the gallbladder). In some embodiments, the cryogenic gas ablation medium 1124 exits the fenestrated nozzle 1174 via the plurality of fenestrations 1175 and ablates the outer surface of the gallbladder lumen once the cryogenic gas ablation medium 1124 upon contact with the tissue.

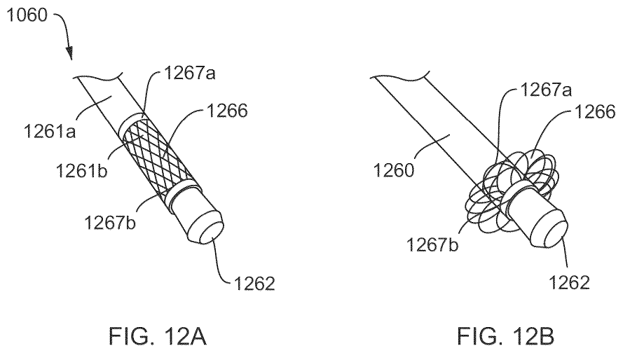

FIGS. 12A-12B are illustrations of an outer shaft 1260 of an ablation system (e.g., a cryoablation device), according to an embodiment. The outer shaft 1260 can include components that are structurally and/or functionally similar to other outer shafts of ablation systems described herein (e.g., outer shaft 260, outer shaft 860, outer shaft 1060, etc.). As shown, the outer shaft 1260 can include an outer tubular member 1261a and an inner tubular member 1261b that are arranged concentrically. Also shown are a lumen 1262 and an expandable structure 1266. FIG. 12A shows the expandable structure 1266 in an undeployed state, while FIG. 12B shows the expandable structure 1266 in a deployed state. As shown, the expandable structure 1266 includes wires arranged in a braided configuration with a proximal ring 1267a and a distal ring 1267b. In some embodiments, at least one of the proximal ring 1267a and the distal ring 1267b can be moved toward the other to induce outward expansion (e.g., deployment) of the expandable structure 1266. In some embodiments, the proximal ring 1267a and the distal ring 1267b can be moved by sliding the tubular members 1261a, 1261b.

In some embodiments, the rings 1267a, 1267b and/or wires of the expandable structure 1266 can be radiopaque to aid in visualizing actuation of the expandable structure 1266 under image guidance (e.g., fluoroscopic imaging, ultrasonic imaging). In some embodiments, the proximal ring 1267a and the distal ring 1267b can be moved via a pull wire, a spring, a sheath, and/or any other suitable mechanism. For example, one or more pull wires can be actuated to move at least one of the proximal ring 1267a and the distal ring 1267b toward the other. In some embodiments, the expandable structure 1266 can be under tension when in the undeployed state (FIG. 12A) and in a relaxed state when in the deployed state (FIG. 12B). In particular, the expandable structure 1266 can be held in tension along an outer surface of the outer shaft 1260, and can be released (e.g., by releasing the hold on one or both ends of the expandable structure 1266, such as by releasing a pull wire, a sheath, etc.) to allow the expandable structure 1266 to self-expand into a deployed state. In some embodiments, the expandable structure 1266 can be composed of a shape memory material, such that they maintain their shape in the deployed state, unless subject to outside force. In some embodiments, the bands can be under tension when in the deployed state and in a relaxed state when in the undeployed state. For example, pushing or moving the outer tubular member 1261a in a distal direction relative to the inner tubular member 1261b can cause the expandable structure 1266 to transition from an undeployed state to a deployed state. As another example, pulling or moving the inner tubular member 1261b in a proximal direction relative to the outer tubular member 1261a can cause the expandable structure 1266 to transition from an undeployed state to a deployed state.

FIGS. 13A-13C show portions of ablation devices with different arrangements of expandable structures and nozzles. The ablation devices described in FIGS. 13A-13C can include components that are structurally and/or functionally similar to other ablation systems and components thereof described herein (e.g., ablation system 100, catheter system 250, control unit 310, cryoablation device 800, cryoablation catheter 1050, etc.). FIG. 13A shows an outer shaft 1360 with an expandable structure 1366 and an inner shaft 1370 with an expandable structure 1376. The inner shaft 1370 is slidable within the outer shaft 1360, such that the inner shaft 1370 can move axially in a direction along arrow 1391 (e.g., in a direction along a longitudinal axis of the outer shaft 1360). The expandable structure 1376 can be coupled to distal portion of the inner shaft 1370. The inner shaft 1370 can be advanced through the outer shaft 1360 until the distal portion of the inner shaft 1370 (e.g., including the expandable structure 1376) is disposed distal to a distal end of the outer shaft 1360. When so positioned, the expandable structure 1376 can be configured to expand into its expanded state, as depicted in FIG. 13A. While not depicted in FIG. 13A, a nozzle can be separately advanced distally from the outer shaft 1360. For example, a separate shaft supporting a nozzle can be advanced through the lumen of the outer shaft 1360 and into a space proximate to the expandable structure 1376. The nozzle can be advanced separately from the expandable structure 1376 such that a user can manipulate a position of the nozzle relative to the expandable structure 1376. Alternatively, in some embodiments, the inner shaft 1370 can include a nozzle. The expandable structure 1376 can include a plurality of bands or wires. In some embodiments, the bands can be composed of a shape memory material, such that they maintain their shape in the deployed state, unless subject to outside force. As described above, in some embodiments, the bands can be under tension when in the undeployed state and in a relaxed state when in the deployed state. Alternatively, the bands can be in a relaxed state when in the undeployed state and under tension when in the deployed state.

FIG. 13B shows an ablation device with an outer shaft 1360′, an inner shaft 1370′, a nozzle 1374′, an expandable structure 1376′, and a hub 1379′. As shown, the expandable structure 1376′ includes a plurality of wires or bands that extends outward and distally from the inner shaft 1370′ and couples to a distal hub 1379′. In some embodiments, the bands extend through the length of the inner shaft 1370′, such that the bands can be advanced and retracted from the proximal end of the inner shaft 1370′. Advancement of the bands can cause expansion of the expandable structure 1376′ to deploy the expandable structure 1376′, and retraction of the bands can pull the expandable structure 1376′ back toward the inner shaft 1370 to return the expandable structure 1376′ to its undeployed state. In some embodiments, the bands of the expandable structure 1376′ can be under tension when in an undeployed state and in a relaxed state when in the deployed state. In other words, the bands can be held in tension such that the expandable structure 1376′ is held in a undeployed or unexpanded state, and releasing the bands can allow the bands to self-expand into the deployed state. In some embodiments, the bands can be moved via an actuator located at a proximal end of the inner shaft 1370′. In some embodiments, the actuator can be activated by pushing a button, moving a slider, releasing a spring, or actuating any other suitable mechanism. In some embodiments, the bands of the expandable structure 1376′ can be under tension when in the deployed state and in a relaxed state when in the undeployed state. In other words, the bands can be advanced or pushed from the proximal end of the inner shaft 1370′ to expand the expandable structure 1376′ into its expanded or deployed state. As shown, the nozzle 1374′ can be located within the expandable structure 1376′. The nozzle 1374′ can be coupled to a lumen that extends through the inner shaft 1370′, such that the nozzle 1374′ can receive and delivery an ablation medium into the body lumen. The nozzle 1374′ can terminate proximal of the hub 1379′. In some embodiments, the nozzle 1374′ can be advanced independently of the expandable structure 1376′ such that its position within the expandable structure 1376′ can be adjusted.

In some embodiments, a sensor can be disposed in the hub 1379′. In some embodiments, the sensor can be a temperature sensor. In some embodiments, the sensor can be a pressure sensor. In some embodiments, when the inner shaft 1370′ is positioned within a gallbladder lumen to deliver the ablation medium (e.g., cryogenic ablation medium), the hub 1379′ can be positioned at or proximate to a cystic duct and can measure a temperature and/or a pressure within the cystic duct. Such measurements can be used to monitor a progress of the ablation procedure and/or operational conditions during the ablation procedure (e.g., for safety).

FIG. 13C shows an ablation device (e.g., cryogenic catheter) with an outer shaft 1360″, an inner shaft 1370″, a nozzle 1374″, an expandable structure 1376″. The ablation device can include a sleeve 1371″ that defines a set of one or more lumens for receiving one or more bands or wires that form the expandable structure 1376″. The bands that form the expandable structure 1376″ can extend from a proximal end beyond a distal end of the sleeve 1371″, such that the bands can be advanced and/or retracted from the proximal end of the sleeve 1371″. In some embodiments, the bands of the expandable structure 1376″ can be under tension when in an undeployed state and in a relaxed state when in the deployed state. In other words, pulling the bands from the proximal end of the sleeve 1371″ can flatten the expandable structure 1376″ and releasing the bands can allow the bands to self-expand into the deployed state. In some embodiments, the bands can be pulled or released from the proximal end of the secondary inner shaft 1371″ via an actuator. In some embodiments, the bands of the expandable structure 1376″ can be under tension when in the deployed state and in a relaxed state when in the undeployed state. In other words, the bands can be advanced or pushed from the proximal end of the secondary inner shaft 1371″ to expand. In some embodiments, the movement of the bands of the expandable structure 1376″ can be caused by an actuator (e.g., a button, slider, motor, spring, etc.).

In some embodiments, the sleeve 1371″ can move in proximal and distal directions along line 1391. In some embodiments, the sleeve 1371″ can act as a pushing mechanism, e.g., to deploy the expandable structure 1376″. For example, in response to pushing the sleeve 1371″ toward the distal end of the inner shaft 1370″, the expandable structure 1376″ can expand outward to a deployed state in a first direction along arrows 1392. In response to pulling the sleeve 1371″ away from or proximally from the distal end of the inner shaft 1370″, the expandable structure 1376″ contracts inward to an undeployed state in the opposite direction along arrows 1392.

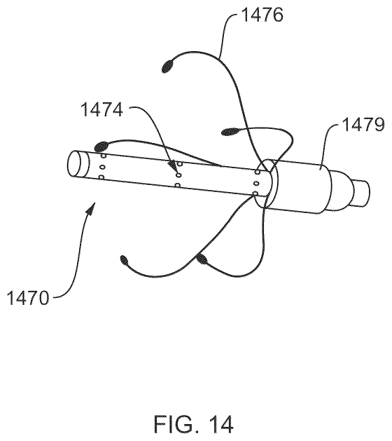

FIG. 14 shows an inner shaft 1470 of an ablation catheter or catheter system with an alternative example of an expandable structure 1476, according to an embodiment. The expandable structure 1476 can include a plurality of wires or splines that are coupled to a hub or shaft at a first end (e.g., a proximal end) and uncoupled at a second, opposite end (e.g., a distal end). In other words, the expandable structure 1476 has an “open” configuration. The inner shaft 1470 can include components that are structurally and/or functionally similar to other ablation systems and components thereof described herein (e.g., the inner shaft 270, inner shaft 870, inner shaft 1170, inner shafts 1370, 1370′, 1370″, etc.). The inner shaft 1470 also includes a nozzle 1474 and a hub 1479. The bands of the expandable structure 1476 can be coupled to the hub 1479 at their proximal end. As shown, the hub 1479 is located on the proximal to the nozzle 1474. While the hub 1479 is depicted being proximal to the nozzle 1474, it can be appreciated that in other embodiments, the hub 1479 can be located distal to the nozzle 1474. Similar to other expandable structures described herein, the expandable structure 1476 can be configured to self-expand (e.g., after being released from being in tension, or after the bands are advanced distal of the hub 1479). In some embodiments, the expandable structure 1476 can be composed of a shape memory material, such that the expandable structure 1476 remains in the expanded state during cryoablation.

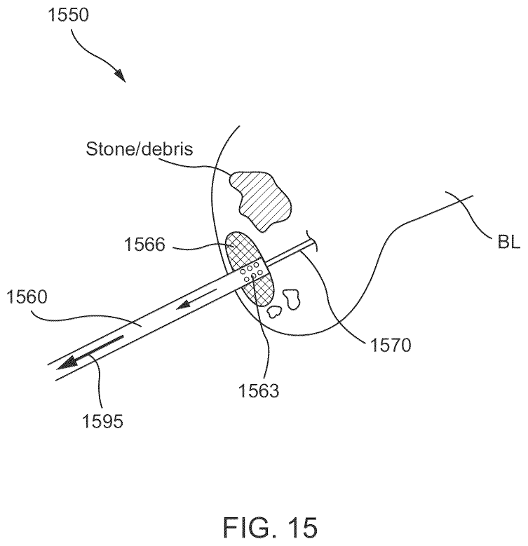

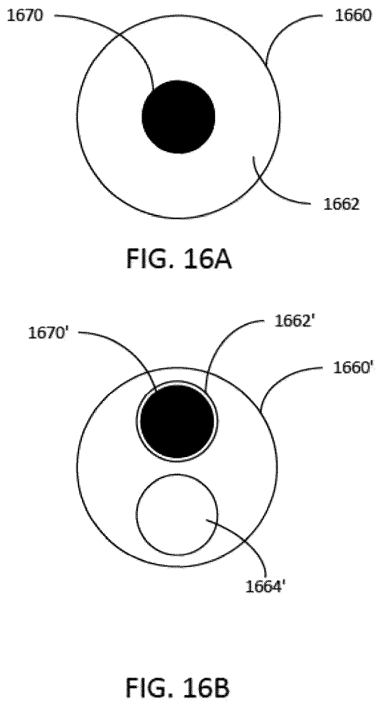

FIGS. 15-16B show catheter systems with evacuation lumens for evacuation of fluids (e.g., gas, liquid) or smaller debris from a body lumen BL, according to various embodiments. FIG. 15 shows an ablation catheter 1550 partially disposed in a body lumen BL, according to an embodiment. The ablation catheter 1550 can include components that are structurally and/or functionally similar to other ablation systems and components thereof described herein (e.g., ablation system 100, catheter system 250, control unit 310, cryoablation device 800, ablation catheter 1050, etc.). The ablation catheter 1550 includes an outer shaft 1560 and an inner shaft 1570. The outer shaft 1560 includes evacuation holes 1563 (e.g., evacuation openings) and an expandable structure 1566. In some embodiments, the evacuation holes 1563 can be inserted into the body lumen BL during ablation, such that fluids or smaller debris can exit the body lumen BL through the evacuation holes 1563 and flow out of the body lumen BL via the outer shaft 1560 along a passageway indicated by arrow 1595. In some embodiments, the expandable structure 1566 can be disposed around the outside of the evacuation holes 1563, such that the expandable structure 1566 acts as a filter to prevent larger pieces of debris from clogging the evacuation holes 1563. In some embodiments, the expandable structure 1566 can have a mesh structure that can aid in filtering debris (e.g., stones, sludge, bile) from entering an evacuation pathway in the outer shaft 1560, creating a reliable pathway to relieve ablation gas and pressure in the body lumen BL. In some embodiments, the debris that exits the body lumen via the evacuation holes 1563 can be solid, liquid, and/or gas. In some embodiments, the evacuation holes 1563 can be fluidically coupled to a lumen that runs along the arrow 1595 inside the outer lumen 1560 and outside the inner lumen 1570. In some embodiments, the expandable structure 1566 can create a reliable pocket for evacuation of ablation gas.

FIGS. 16A-16B show cross sections of outer shafts 1660, 1660′ with evacuation lumens, according to various embodiments. FIG. 16A includes an outer shaft 1660 and an inner shaft 1670. The outer shaft 1660 and inner shaft 1670 can include components that are structurally and/or functionally similar to other ablation systems and components thereof described herein (e.g., outer shaft 260, inner shaft 270, outer shaft 860, inner shaft 870, outer shaft 1060, inner shaft 1070, outer shaft 1260, etc.). As shown in FIG. 16A, the outer shaft 1060 can include a lumen 1662 within which the inner shaft 1070 is disposed. During an ablation procedure, the space between an outer surface of the inner shaft 1070 and an inner surface of the outer shaft 1060 can define an evacuation channel or passageway. In some embodiments, debris can flow into evacuation holes (e.g., evacuation holes 1563) within a body lumen and flow through the evacuation channel to exit the body lumen.

FIG. 16B depicts an alternative arrangement of lumens within an outer shaft 1670′ of an ablation catheter (e.g., a cryoablation device). As shown, the outer shaft 1670′ can include a separate lumen 1664′ that is designated for evacuation of content (e.g., solids, fluids, etc.) from within the body lumen BL. In some embodiments, the lumen 1664′ can be fluidically coupled to evacuation holes (e.g., evacuation holes 1563) that can be disposed in the body lumen. In some embodiments, the lumen 1664′ can provide a flow path for fluid and/or debris to exit the body lumen. Similar to other outer shafts, the outer shaft 1660′ can define a lumen 1662′ that can receive an inner shaft 1670′ and be used to guide the inner shaft 1670′ into the body lumen.

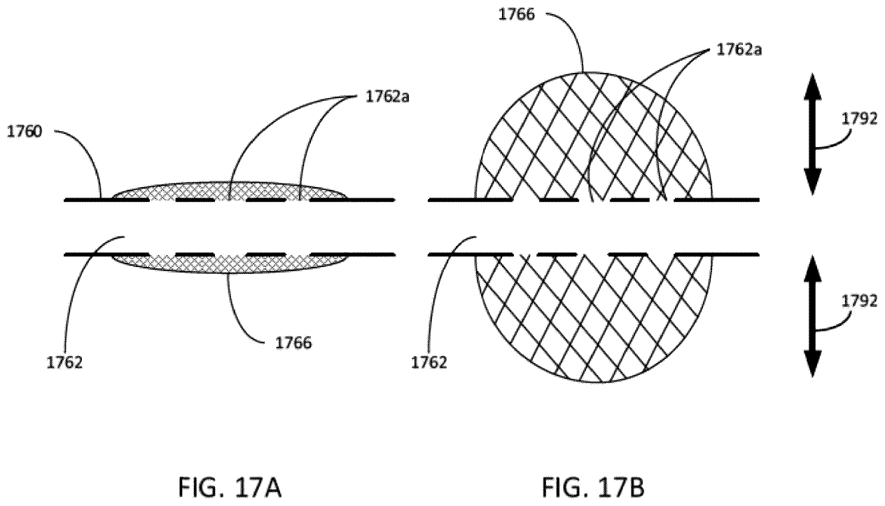

FIGS. 17A-17B show an outer shaft 1760 of an ablation catheter (e.g., a cryoablation device) with an expandable structure 1766, according to an embodiment. The outer shaft 1760 includes evacuation holes 1762a and an evacuation lumen 1762. The outer shaft 1760 can include components that are structurally and/or functionally similar to other ablation systems and components thereof described herein (e.g., outer shaft 260, outer shaft 860, outer shaft 1060, inner shaft 1070, outer shaft 1660, etc.). FIG. 17A shows the expandable structure 1766 in an undeployed (i.e., unexpanded) state, while FIG. 17B shows the expandable structure 1766 in a deployed state. As shown, the expandable structure 1766 is disposed on the outer shaft 1760, such that the expandable structure 1766 covers the evacuation holes 1762a. Upon deployment of the expandable structure 1766, the expandable structure 1766 expands outward in a direction indicated by arrows 1792. In some embodiments, the expandable structure 1766 can create a mesh or include perforations or openings, such that in its expanded state, the expandable structure 1766 can act as a filter for fluids and/or debris entering the evacuation holes 1762a. In such a case, the expandable structure 1766 can act as a filter and prevent any debris large enough to clog the evacuation holes 1762a from entering the evacuation holes 1762a.

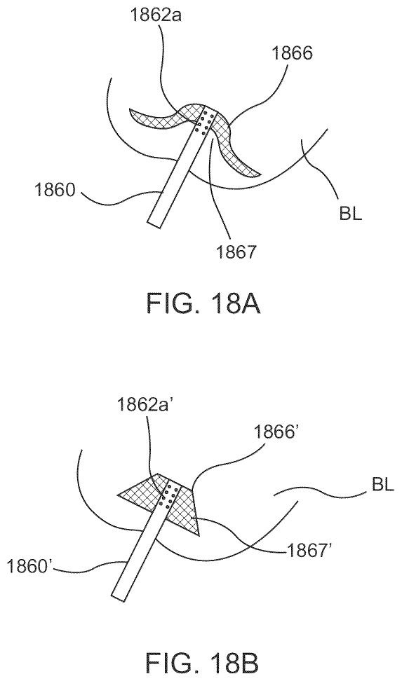

FIGS. 18A-18B show different arrangements of expandable structures of outer shafts of an ablation catheter, according to embodiments. FIGS. 18A-18B depict outer shafts 1860, 1860′ of an ablation catheter (e.g., a cryoablation device), disposed in a body lumen BL. The outer shaft 1860, 1860′ can include components that are structurally and/or functionally similar to other ablation systems and components thereof described herein (e.g., outer shaft 260, outer shaft 860, outer shaft 1060, inner shaft 1070, outer shaft 1660, outer shaft 1760, etc.). FIG. 18A shows the outer shaft 1860 with an expandable structure 1866, evacuation holes 1862a, and an evacuation pocket 1867. As shown, the evacuation holes 1862a are disposed in the body lumen BL when the outer shaft 1860 is positioned for an ablation procedure. The expandable structure 1866 can be configured to expand to form a curved or concave structure that defines an evacuation pocket 1867 around the evacuation holes 1862a. Stated differently, the expandable structure 1866 can form a shape similar to an umbrella that blocks debris from entering evacuation holes 1862a.

FIG. 18B shows the outer shaft 1860′ with an expandable structure 1866′, evacuation holes 1862a′, and an evacuation pocket 1867′. As shown, the evacuation holes 1862a′ are disposed in the body lumen BL. The expandable structure 1866′ partially covers the evacuation holes 1862a′ forming the evacuation pocket 1867′. As shown, the evacuation pocket 1867′ is expanded such that it has a flat or substantially flat proximal side. This shape can form a secure engagement with the walls at the entry point of the body lumen BL to stabilize the outer shaft 1860′ within the body lumen. This shape can ensure better retention of the outer shaft 1860′ within in a body lumen such as, for example, a gallbladder.

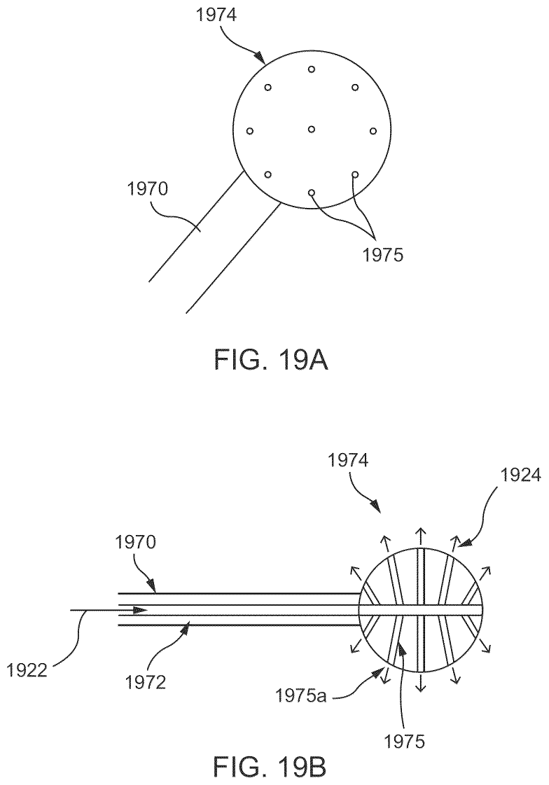

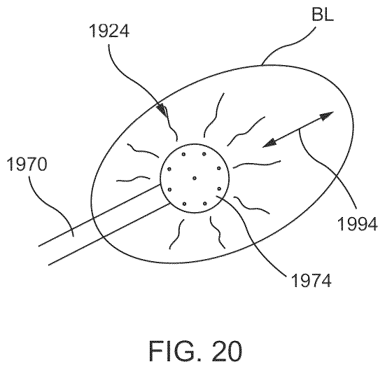

FIGS. 19A-19B and FIG. 20 show example views of an inner shaft 1970 with a nozzle 1974 (e.g., a dispersion nozzle) located at the distal end of the inner shaft 1970, according to various embodiments. FIG. 19A illustrates a perspective view of the inner shaft 1970 while FIG. 19B illustrates a cross-sectional side view of the inner shaft 1970. The inner shaft 1970 can include components that are structurally and/or functionally similar to other ablation systems and components thereof described herein (e.g., inner shaft 270, inner shaft 870, inner shaft 1070, inner shaft 1670, etc.).

In some embodiments, the inner shaft 1970 can include a long catheter body with at least one delivery lumen 1972 that carries liquid ablation medium 1922 and terminates into the dispersion nozzle 1974. In some embodiments, the geometry of the dispersion nozzle 1974 is spherical and includes a series of holes 1975 that span from the outer diameter of the dispersion nozzle 1974 to the supply lumen 1972. In some embodiments, the dispersion nozzle 1974 uses liquid nitrous oxide as the ablation medium that undergoes a phase-change where the geometry of each hole 1975 intersects the outer surface of the dispersion nozzle 1974 (a phase-change interface 1975a). In other words, the liquid ablation medium 1922 transitions into a gas ablation medium 1924 near the outer surface of the dispersion nozzle 1974. In some embodiments, the holes 1975 are sufficiently small in size (e.g., on the order of about 0.0005″-0.004″) to withstand the high pressures needed to keep a nitrous oxide in its liquid form until the cryogen reaches the desired phase-change interface 1975a. In some embodiments, the phase-change interface 1975a is controlled by a pressure drop (e.g., atmospheric venting) relative to the supply pressure of the liquid ablation medium 1922. In some embodiments, the phase change occurs when the liquid nitrous oxide is exposed to the near atmospheric pressure in a body lumen BL or other desired ablation area. In some embodiments, the phase change occurs at the wall of the body lumen BL; therefore fluid ablation medium can be delivered into the body lumen BL and contact the wall of the body lumen BL and phase change into a gas ablation medium. In such cases, ablation can occur at the liquid-gas phase change interface. As shown, the holes 1975 that are located on the proximal side of the nozzle 1974 are angled (e.g., angled proximally relative to a longitudinal axis of the inner shaft 1970), such that the gas ablation medium 1924 is dispensed at an angle toward a proximal region of a body lumen. The holes 1975 that are located on the distal side of the nozzle 1974 are angled (e.g., angled distally relative to a longitudinal axis of the inner shaft 1970), such that the gas ablation medium 1924 is dispersed at an angle toward a distal region of a body lumen. This angled configuration of the holes 1975 can aid in increasing distribution of the gas ablation medium 1924 throughout the body lumen.

Although illustrated as a spherical configuration, the geometry of the dispersion nozzle 1974 can be a cube, cone, cylinder, triangular prism, torus, helix, ovoid, or any other three dimensional (3D) body with sufficient structure to enable the delivery of ablation medium. In some embodiments, the dispersion nozzle 1974 can be made from metal, polymer, ceramic, or other structural material. In some embodiments, the maximum diameter of the distal geometry is sufficiently small to slide through an access catheter. In some embodiments, the dispersion nozzle 1974 can be expanded (e.g. inflated) to achieve a larger shape than the diameter of the access catheter.

FIG. 20 illustrates how the gas ablation medium 1924 is uniformly dispersed from the dispersion nozzle 1974 to the walls of the body lumen BL during use. In some embodiments, the inner shaft 1970 can move freely in an axial direction (i.e., along the axis indicated by line 1994) in order to sufficiently treat the walls of the body lumen BL with gas ablation medium 1924 throughout the axial length of the body lumen BL.

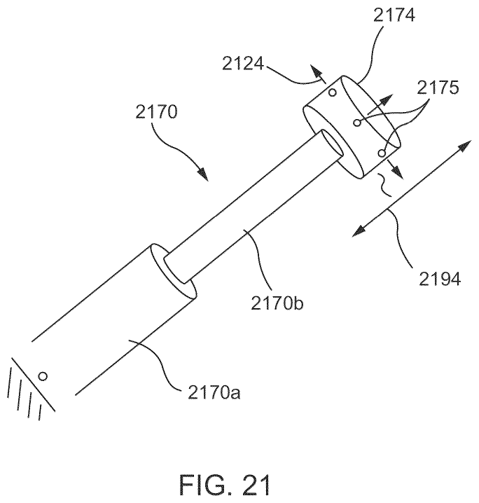

FIG. 21 shows an inner shaft 2170 with an actuated nozzle 2174 located near the distal end of the inner shaft 2170, according to an embodiment. The inner shaft 2170 can include components that are structurally and/or functionally similar to other ablation systems and components thereof described herein (e.g., inner shaft 270, inner shaft 870, inner shaft 1070, inner shaft 1670, inner shaft 1970, etc.). In some embodiments, gas ablation medium 2124 can be expelled via holes 2175 on the actuated nozzle 2174. In some embodiments, the inner shaft 2170 can include an inner shaft body 2170a and a linear rail component 2170b. In some embodiments, the actuated nozzle 2174 can be attached to the linear rail component 2170b. In some embodiments, the linear rail component 2170b allows the actuated nozzle 2174 to move axially (i.e., along the line indicated by arrow 2194) in response to a driving force. In some embodiments, the linear rail component 2170b allows the actuated nozzle 2174 to move non-linearly along its central axis in response to a driving force.

In some embodiments, the driving force is either manually or automatically applied such as via a control unit (e.g., control unit 110). In some embodiments, the driving force can be manually or automatically be applied using a stiff drive wire system, a flexible drive cable system, a mating gear drive system, a rack-and-pinion system, a screw-drive mechanism, a pneumatic actuator system, an electromagnetic coil system, a hydraulic actuator system, or any other type of system as can be appreciated. In some embodiments, the driving force is the user's grip force, pull force, twist force or squeeze force. In some embodiments, the driving force can be electromechanical, such as the use of electrical current to drive an AC/DC motor or the use of electromagnetic fields.

In some embodiments, the linear rail component 2170b can be fixed or nearly fixed by a distal and proximal feature to the linear rail component 2170b. In some embodiments, the linear rail component 2170b can be fixed or nearly fixed by only a proximal feature to the rail. In some embodiments, the linear rail component 2170b can be fixed or nearly fixed by only a distal feature to the linear rail component 2170b. In some embodiments, the distal feature can be a cystic duct occlusion mechanism. In some embodiments, the proximal feature is an access catheter lumen.

In some embodiments, the actuated nozzle 2174 can be similar to the nozzles described in FIGS. 19A, 19B, and FIG. 20, according to various embodiments. In some embodiments, the diameter of the hole or holes 2175 located on the actuated nozzle 2174 can vary in diameter, relative to their position. In some cases, the holes 2175 can be “tapered” or increase/decrease in diameter, along the geometry, to deliver a constant mass flow rate of gas ablation medium 2124 and combat the effects of pressure drop in an ablation supply lumen.

In some embodiments, the size, shape, and number of holes 2175 emanating from the supply lumen will determine the spray pattern, spray velocity, and spray uniformity of the ablation medium. In some embodiments, some of the holes 2175 are optimized to target close targets (e.g., 0-0.5 cm). In some embodiments, some of the holes 2175 are optimized to target distant targets (e.g., greater than 0.5 cm).

In some embodiments, the actuated nozzle 2174 is able to spin along its central axis, rotating the holes 2175 relative to their starting position. In some embodiments, the actuated nozzle 2174 is able to spin between 0-360 degrees or any inclusive range. In at some embodiments, the rotating actuated nozzle 2174 allows for greater coverage of ablation medium delivery.

In some embodiments, the actuated nozzle 2174 can be fixed relative to the distal end of the linear rail component 2170b and can move with the displacement of the linear rail component 2170b by the driving force.

In some embodiments, the linear rail component 2170b can facilitate either concentric or non-concentric movement of the actuated nozzle 2174 between about 0-10 cm or any inclusive range in response to a driving force.

FIG. 22 shows an inner shaft 2270 with an actuated nozzle 2274 located near the distal end of the inner shaft 2270, according to an embodiment. The inner shaft 2270 can include components that are structurally and/or functionally similar to other ablation systems and components thereof described herein (e.g., inner shaft 270, inner shaft 870, inner shaft 1070, inner shaft 1670, inner shaft 1970, inner shaft 2170, etc.). In some embodiments, gas ablation medium 2224 can be expelled via holes 2275 on the actuated nozzle 2274. In some embodiments, the inner shaft 2270 can include an inner shaft body 2270a and a linear rail component 2270b. In some embodiments, the actuated nozzle 2274 can move in an axial direction (i.e., along the line indicated by arrow 2294) along the linear rail component 2270b in response to a driving force. In some embodiments, the inner shaft 2270, the actuated nozzle 2274, the holes 2275, the inner shaft body 2270a, and the linear rail component 2270b can be the same or substantially similar to the inner shaft 2170, the actuated nozzle 2174, the holes 2175, the inner shaft body 2170a, and the linear rail component 2170b, as described above with reference to FIG. 21.

The inner shaft 2270 also includes an occluder 2279. The occluder 2279 can be configured to occlude or close an opening or lumen outlet into nearby anatomical structures from a body lumen. For example, in the case where the body lumen is a gallbladder lumen, the occluder 2279 can be configured to occlude a cystic duct. The occluder 2279 can be coupled to and/or detachable from the inner shaft 2270. In operation, the occluder 2279 can be coupled to a distal end of the inner shaft 2270. In some embodiments, the occluder 2279 can be coupled to the linear rail component 2270b. The inner shaft 2270 can be navigated into the body lumen. The inner shaft 2270 can be manipulated to position the occluder 2279 at an opening out of the body lumen (e.g. outlet lumen such as a cystic duct). The occluder 2279 can then be decoupled or ejected from the inner shaft 2270, allowing the occluder 2279 to be placed in the opening. The occluder 2729 can subsequently be fixed in place, e.g., via volume expansion of the occluder 2279, external threads, friction fit, adhesion, or other suitable fixation mechanism. Further details of suitable occluders such as, for example, plugs, are described in International Patent Application No. PCT/US2019/017112, incorporated herein by reference.

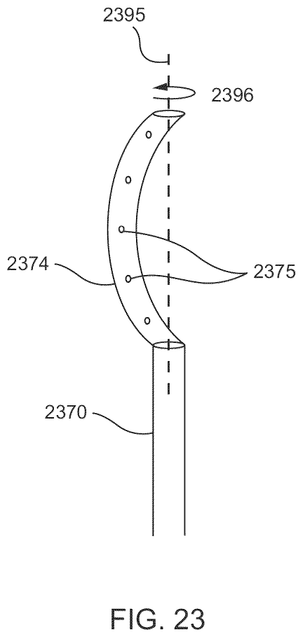

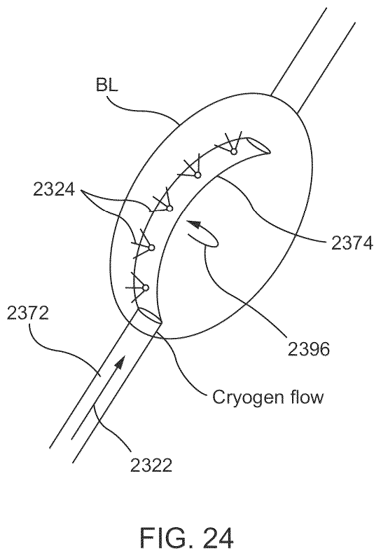

FIGS. 23-24 show an inner shaft 2370 having a nozzle 2374 with a bowed design that can increase the effective spray area of an ablation medium. The inner shaft 2370 can include components that are structurally and/or functionally similar to other ablation systems and components thereof described herein (e.g., inner shaft 270, inner shaft 870, inner shaft 1070, inner shaft 1670, inner shaft 1970, inner shaft 2170, inner shaft 2270, etc.). In some embodiments, the nozzle 2374 has at least one bowed segment that contains at least one hole 2375 emanating from a supply lumen. In some embodiments, at the nozzle 2374 can rotate along its central axis 2395 (i.e., along arrow 2396) to uniformly deliver an ablation medium to the surface of a body lumen. FIG. 24 shows the inner shaft 2370 disposed in a body lumen BL. As shown, liquid ablation medium 2322 exits the nozzle 2374 via the holes 2375 and undergoes a phase change to become a gas ablation medium 2324.

In at least one embodiment, the diameter of the one or more holes 2375 located on the nozzle 2374 can vary in diameter, relative to their distance along the nozzle 2374. In some cases, the holes can be “tapered” or increase/decrease in diameter, between the proximal and distal end of the nozzle 2374, to deliver a constant mass flow rate of ablation medium and combat the effects of pressure drop in the supply lumen.

In some embodiments, the size, shape, and number of holes 2375 emanating from the supply lumen will determine the spray pattern, spray velocity, and spray uniformity of the ablation medium. In some embodiments, the entire nozzle 2374 can rotate and/or slide longitudinally, relative to its central axis 2395. In some embodiments, some of the holes 2375 are optimized to target close targets. In some embodiments, some of the holes 2375 are optimized to target distant targets.

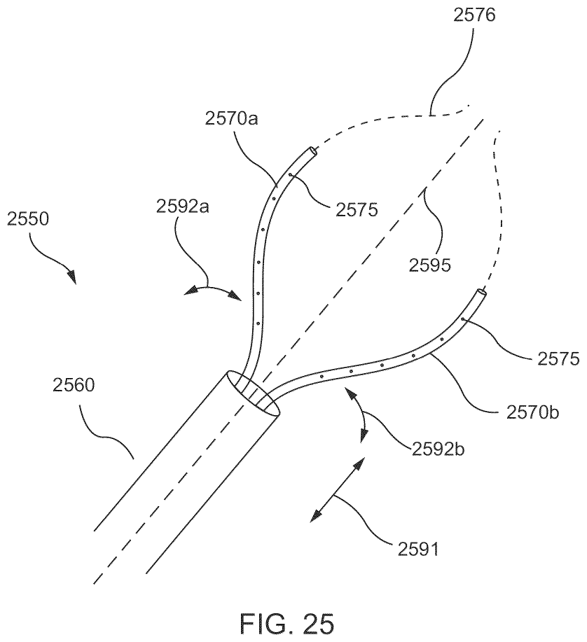

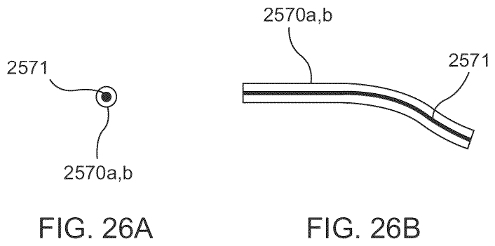

FIGS. 25-26B show an ablation catheter 2550 with a collapsible cryogen dispersion nozzle, according to an embodiment. The ablation catheter 2550 can include components that are structurally and/or functionally similar to other ablation systems and components thereof described herein (e.g., ablation system 100, catheter system 250, control unit 310, cryoablation device 800, ablation catheter 1050, etc.). The ablation catheter 2550 includes an outer shaft 2560, a first inner shaft 2570a, and a second inner shaft 2570b. In some embodiments, the ablation catheter 2550 can include an expandable structure 2576. In some embodiments, the inner shafts 2570a, 2570b (collectively referred to as inner shafts 2570) can be coupled to the expandable structure 2576. In some embodiments, the inner shafts 2570 can be uncoupled at their distal ends. In other words, the ablation catheter 2550 can be without an expandable structure 2576. The inner shafts 2570 include holes 2575 for the delivery of ablation medium. In some embodiments, the inner shafts 2570a can move along the line indicated by arrow 2591. In some embodiments, the inner shafts 2570 can be rotated around a central axis 2595 (i.e., along paths indicated by arrows 2592a, 2592b). As shown, the ablation catheter 2550 includes two inner shafts 2570. In some embodiments, the ablation catheter 2550 can include 3, 4, 5, 6, 7, 8, 9, 10, 11, 12, 13, 14, 15, 16, 17, 18, 19, 20, or more inner shafts 2570. As shown, the inner shafts 2570, extend a partial length of the expandable structure 2576. In some embodiments, the inner shafts 2570 can extend the entire length of the expandable structure 2576, such that the inner shafts 2570 are coupled together at a distal end of the ablation catheter 2550.

In some embodiments, at least of the inner shafts 2570 forms a bowed shape along the central axis 2595 that extends to a maximum radial dimension and converges back towards the central axis 2595 to bring the holes 2575 closer to the target ablation site. In some embodiments, the ablation catheter 2550 uses liquid nitrous oxide as an ablation medium and is configured such that the phase-change interface of the ablation medium is located on the outer surface of the inner shafts 2570.

In some embodiments, the inner shafts 2570 can be spring-loaded and can collapse to be delivered through a smaller diameter delivery lumen, relative to the nominal expanded diameter of the inner shafts 2570.

FIGS. 26A-26B show the inner shafts 2570 in greater detail. FIG. 26A shows a cross-sectional view of the inner shafts 2570, while FIG. 26B shows a side view of the inner shafts 2570. In some embodiments, the ablation catheter 2550 can be constructed with a pre-shaped core 2571 within the inner shafts 2570 that exerts a return force when subjected to mechanical stress, thermal energy, electrical current, or light. In some embodiments, the pre-shaped core 2541 can be made from an alloy metal, such as Nitinol or spring steel. In some embodiments, the pre-shaped core 2571 can be made from a polymer, such as acrylonitrile butadiene styrene (ABS). In some embodiments, the inner shafts 2570 can be driven to an expanded conformation by a mechanical driving force, such as rack and pinion gear system, a cable drive system, or electromechanical control system. In some embodiments, the inner shafts 2570 can be actuated along a linear or radial pathway to increase distribution of cryogen from the inner shafts 2570.

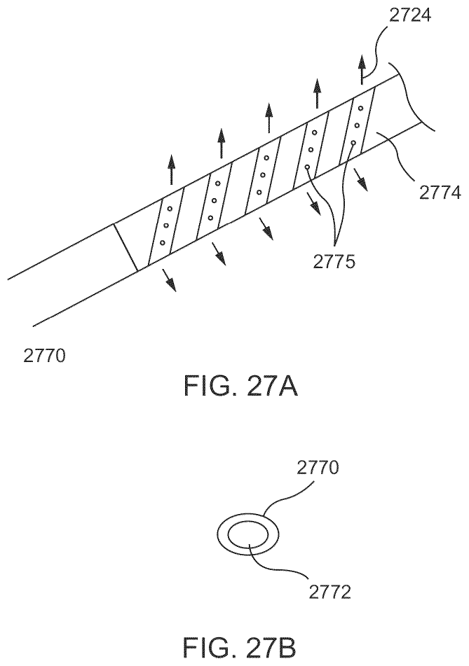

FIGS. 27A-27B show an inner shaft 2770 having a spiral nozzle 2774 with a number of holes 2775 emanating from at least one continuous supply lumen 2772. FIG. 27A shows a side view of the inner shaft 2770, while FIG. 27B shows a cross-sectional view of the inner shaft 2770. The inner shaft 2770 can include components that are structurally and/or functionally similar to other ablation systems and components thereof described herein (e.g., inner shaft 270, inner shaft 870, inner shaft 1070, inner shaft 1670, inner shaft 1970, inner shaft 2170, inner shaft 2270, etc.). Gas ablation medium 2724 is shown exiting the inner shaft 2770 via the holes 2775. In some embodiments, the spiral nozzle 2774 can be mounted around a structural body that holds the spiral conformation of the spiral nozzle 2774. In some embodiments, the holes 2775 can vary in size, shape, and location depending on the desired spray pattern. In some embodiments, the holes 2775 can “taper” or increase/decrease along the nozzle 2774, relative to their distance along the nozzle 2774, to maintain a desired mass flow rate along each of the holes 2775. In some embodiments, the aforementioned design allows for a minimal distance between the phase change surface and the supply lumen 2772, so as to minimize variability in spray patterns between holes.

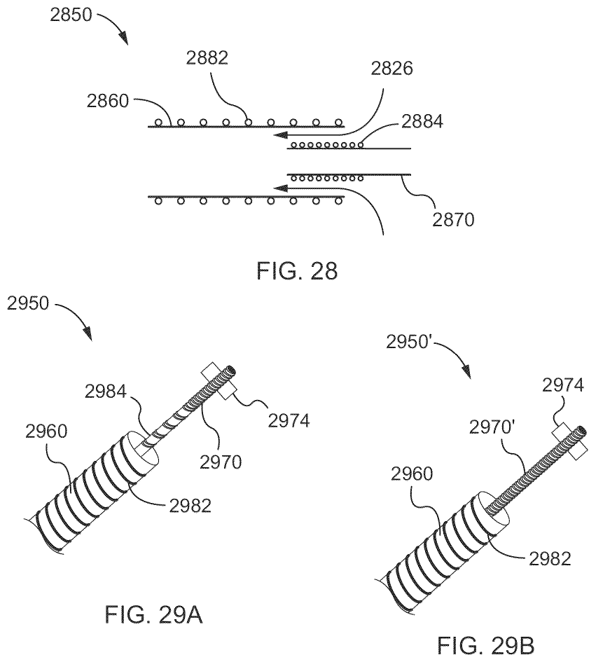

FIG. 28 shows an ablation catheter 2850 with an outer shaft 2860 and an inner shaft 2870. The ablation catheter 2850 can include components that are structurally and/or functionally similar to other ablation systems and components thereof described herein (e.g., ablation system 100, catheter system 250, control unit 310, cryoablation device 800, ablation catheter 1050, etc.). Ablation medium 2826 can be evacuated from a body lumen through the outer shaft 2860. In some embodiments, heating coils 2882 can be deployed around the outside of the outer shaft 2860. In some embodiments, heating coils 2884 can be deployed around the outside of the inner shaft 2870. According to various embodiments of the present disclosure, the cryoablation devices of the present disclosure are designed to leverage the phase-change properties of liquid nitrous oxide (N₂O or LN₂O), to induce cryoablation temperatures (e.g., about −80 C) at the target tissue interface. N₂O is a clear liquid at ambient temperatures and high pressures (>650 psi), but undergoes a phase change from liquid to gas when it experiences a sufficient pressure drop, resulting in an endothermic reaction that produces a refrigerant property. Further, while liquid nitrous oxide affords a unique refrigerant property that is well suited for cryoablation applications, it can present safety issues as the volume of the gas can increase 600-fold or more during the phase change, creating a source of pressure build up within the lumen. In order to combat this risk, the cryoablation devices of the present disclosure can be designed to utilize a passive evacuation management system to vent cryogen gas out of the body during the procedure. For example, systems, devices, and methods described herein can allow flow of ablation medium through a concentric lumen space between an (e.g., inner shaft 2870) and an outer shaft (e.g., outer shaft 2860) of an ablation catheter. Pressure driven flow can cause the ablation medium to enter the lumen space between the inner shaft and the outer shaft and exit out an exhaust port at a proximal end of ablation catheter.

In addition, liquid nitrous oxide has a melting point within a few degrees Celsius of its boiling point, i.e., a small margin exists between its gas phase and solid phase. Such can lead to solid nitrous oxide ice buildup if the pressure and temperatures within the outer shaft and the inner shaft are not controlled properly. Solid nitrous ice buildup, in conjunction with remnant fluid within the gallbladder, can lead to clogging of the evacuation lumen in certain circumstances. This can cause pressure build-up within the gallbladder lumen and is a safety concern. To directly combat icing of the evacuation lumen, the heating coils 2882, 2884 can be applied to melt or evaporate ice build-up.

FIGS. 29A-29B show views of an ablation catheter 2950 with a catheter heating system configured to combat ice build-up in the evacuation lumen, according to various embodiments of the present disclosure. The ablation catheter 2950 can include components that are structurally and/or functionally similar to other ablation systems and components thereof described herein (e.g., ablation system 100, catheter system 250, control unit 310, cryoablation device 800, ablation catheter 1050, etc.). As shown, the ablation catheter 2950 includes an outer shaft 2960 and an inner shaft 2970. The inner shaft 2970 includes a nozzle 2974. In some embodiments, a heating coil 2982 can be wrapped around (e.g., disposed around) the outside of the outer shaft 2960. In some embodiments, a heating coil 2984 can be wrapped around (e.g., disposed around) the outside of the inner shaft 2970. In particular, FIG. 29A illustrates an example view of a catheter heating system designed to defrost cryogen ice build-up within a lumen. The catheter heating system of FIG. 29A includes a multi-surface heating system in which the outer face of the outer shaft 2960 and the outer face of the inner shaft 2970 are heated to vaporize ice-build up and maintain the patency of the annular space between the two shafts. FIG. 29B shows a ablation catheter 2950′ with a single surface heating system in which the outer face of the outer shaft 2960 is heated via heating coil 2982 to vaporize ice-build up and maintain the patency of the annular space between the inner shaft 2970 and the outer shaft 2960.

In some embodiments, the heating coils 2982, 2984 can include a resistive heating element, such as, for example, a resistive wire, that transfers electrical energy into heat, thereby conductively heating nearby bodies. In some embodiments, the resistive heating wire is wrapped around the outer circumference of the outer shaft 2960. In some embodiments, the resistive heating wire is wrapped around the inner circumference of the outer shaft 2960. In at least one embodiment, the resistive heating wire is embedded within the outer shaft 2960 wall material.

In some embodiments, the resistive heating wire is wrapped in a helical coil configuration with about 0-1″ pitch spacing, including all subranges and values in between. In some embodiments, the resistive heating wire is wrapped in a helical coil configuration with a fixed pitch. In another embodiment, the resistive heating wire is wrapped in a helical coil configuration with a variable or “progressive fix” such that the sections of tighter coil pitch are located closer to the distal end of the outer shaft 2960. In the aforementioned configuration, the tighter pitch section enables greater heat density, compared to looser pitch sections, thereby heating the bodies surrounding the tighter pitch section more. Such can localize the heating energy of the coil and minimize competing effects on the therapy.

In some embodiments, the outer shaft 2960 can be polymer, metal, ceramic, or composite or any combination of. In some embodiments, a metal or high thermal conductance material can span parts and the entirety of the outer shaft 2960 wall thickness and circumference to increase the heat transfer rate to the desired heating target. In some embodiments, the delivery lumen can have a metal segment near the distal end of the outer shaft 2960 to concentrate the effect of the heating coil 2982. In some embodiments, an insulating material can be used to electrically and/or thermally insulate the heating coil 2982 from surrounding bodies.

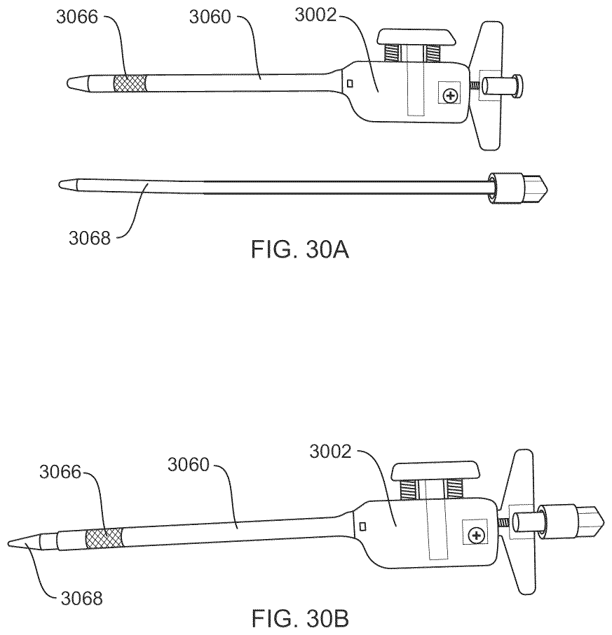

FIGS. 30A-33 show an ablation catheter 3050 in various stages of assembly. FIGS. 30A-30B show an outer shaft 3060 and a dilator 3068, according to an embodiment. The ablation catheter 3250 can include components that are structurally and/or functionally similar to other ablation systems and components thereof described herein (e.g., ablation system 100, catheter system 250, control unit 310, cryoablation device 800, ablation catheter 1050, etc.). The outer shaft 3060 includes a handle assembly 3002 and an expandable structure 3066. In some embodiments, the deployment of the expandable structure 3066 can be controlled by the handle assembly 3002. FIG. 30A shows the outer shaft 3060 separated from the dilator 3068. FIG. 30B shows the dilator 3068 secured in the outer shaft 3060. In some embodiments, the dilator 3068 can be used to securely fit the outer shaft around a guidewire during insertion into a body lumen, as described above with reference to FIGS. 6A-6B. In some embodiments, the dilator 3068 can be secured in the outer shaft 3060 via a threading.

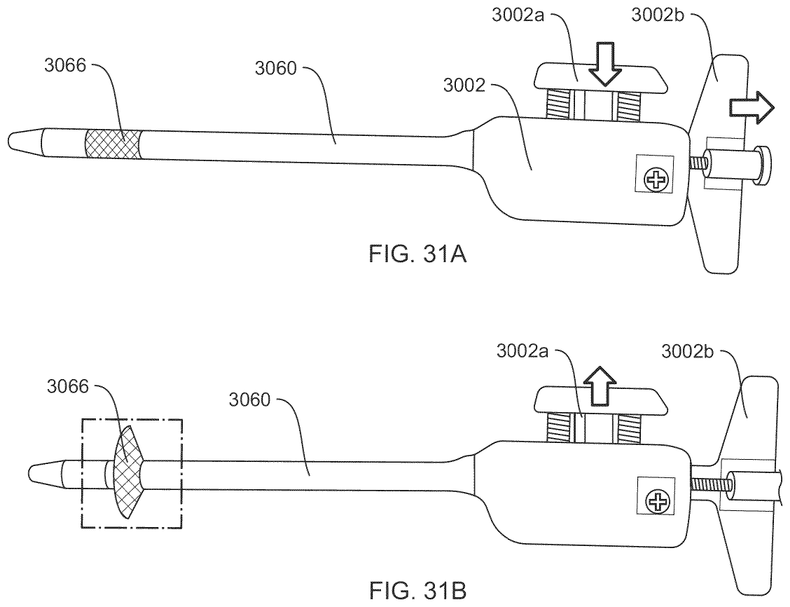

FIGS. 31A-31B show the outer shaft 3060 with an expandable structure 3066 in an undeployed state (FIG. 31A) and a deployed state (FIG. 31B), according to an embodiment. As shown, the handle assembly 3002 includes a button 3002a and a handle 3002b. In some embodiments, pushing the button 3002a can unlock the mechanism that controls the deployment of the expandable structure 3066. After the mechanism is unlocked, the handle 3002b can be pulled to actuate the expandable structure 3066 into the deployed state. Once the expandable structure 3066 is in the deployed state, the button 3002a can be released to lock the expandable structure 3066 in the deployed state.

FIGS. 32A-32C are illustrations of an inner shaft 3070 with an actuator handle assembly 3004, according to an embodiment. The actuator handle assembly 3004 includes an actuator handle 3004a. The inner shaft 3070 includes a nozzle 3074, an expandable structure 3076, and a hub 3079. The nozzle 3074 includes holes 3075a, 3075b, 3075c (collectively referred to as holes 3075). As shown, the holes 3075a in a proximal position are angled, such that an ablation medium exits the nozzle 3074 in a slightly proximal direction. As shown, the holes 3075c in a distal position are angled, such that an ablation medium exits the nozzle 3074 in a slightly distal direction. Advantages of such an angled configuration of the holes 3075 are described above with reference to the holes 1975 in FIGS. 19A-19B.

As shown, FIG. 32A shows detail of the expandable structure 3276, while FIG. 32B shows the actuator handle 3004a positioned such that the expandable structure 3076 is in a deployed state, and FIG. 32C shows the actuator handle 3004a positioned such that the expandable structure 3076 is in an undeployed state. In some embodiments, the actuator handle 3004a can be fixed in position by magnets. For example, the actuator handle assembly 3004 can include magnets on both a proximal side and a distal side, and the actuator handle 3004a can include magnets such that the actuator handle 3004a is attracted to the proximal side and the distal side of the actuator handle assembly 3004. In such a case, the actuator handle 3004a can be more attracted to whichever side of the actuator handle assembly 3004 is closer.

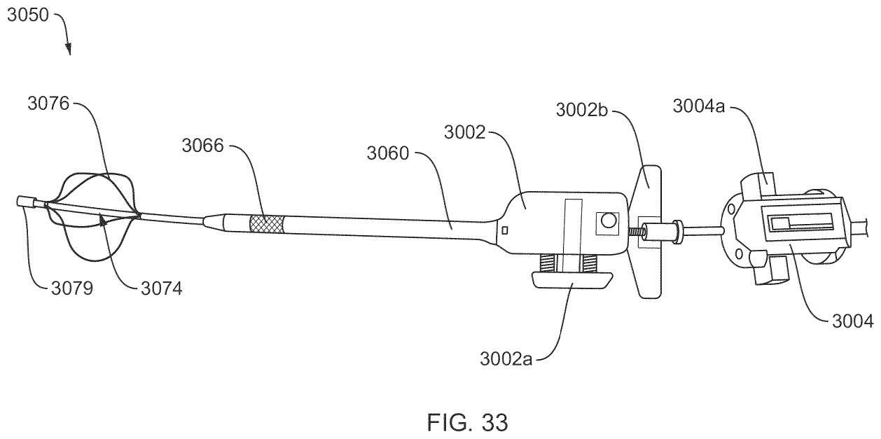

FIG. 33 shows an assembled ablation catheter 3050, with the inner shaft 3070 disposed within and/or coupled to the outer shaft 3060. As shown, the expandable structure 3066 is in the undeployed state and the expandable structure 3076 is in the deployed state. As shown, the actuator handle 3004a is coupled to the actuator handle assembly 3004. In some embodiments, the actuator handle 3004a can be coupled to the actuator handle assembly 3004. In some embodiments, the button 3002a can be disposed in the handle assembly 3002. In some embodiments, the button 3002a can be coupled to the handle assembly 3002. In some embodiments, the handle 3002b can be disposed in the handle assembly 3002. In some embodiments, the handle 3002b can be coupled to the handle assembly 3002.

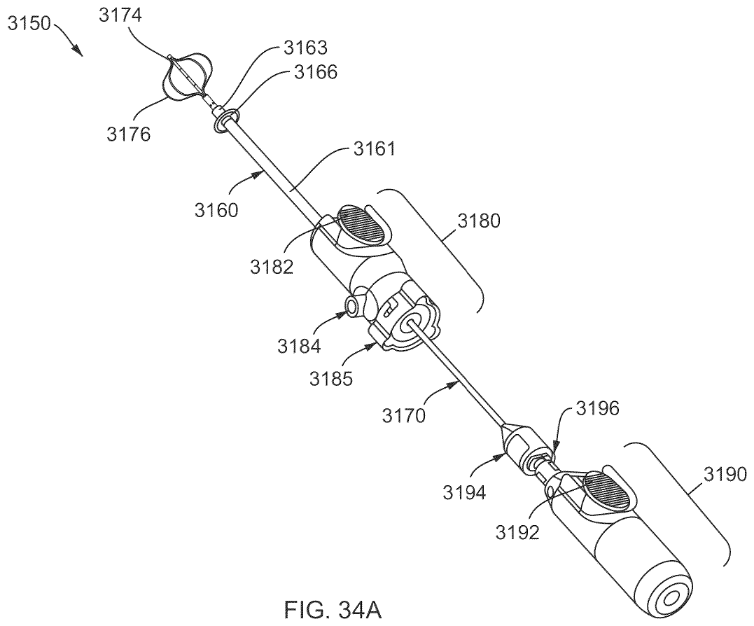

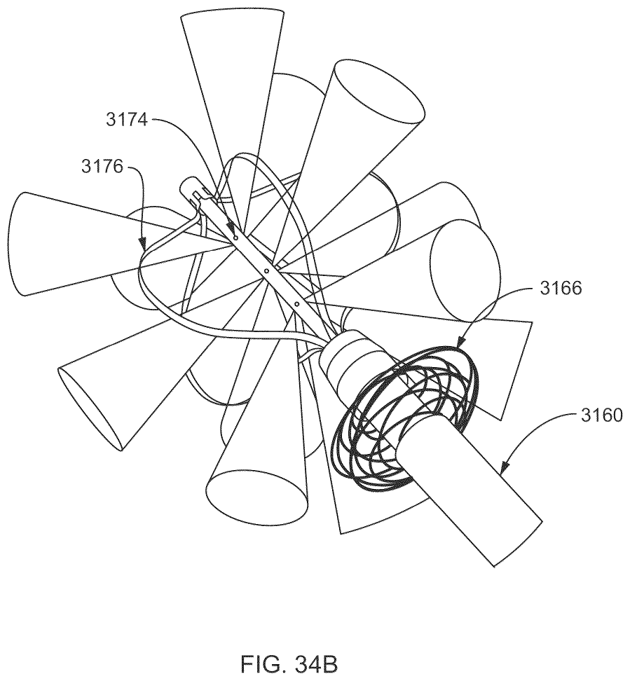

FIGS. 34A-34B shows an ablation system 3150 with a handle assembly including multiple handles, according to an embodiment. FIG. 34A shows the ablation system 3150, while FIG. 34B shows a spray pattern of nozzles of the ablation system 3150. As shown, the ablation system 3150 includes an outer shaft 3160, an expandable structure 3166 (e.g., retention mechanism), an inner shaft 3170, a nozzle 3174, an expandable structure 3176 (e.g., an expandable body or cage), an outer shaft (introducer or access sheath) handle 3180, an outer shaft (introducer or access sheath) handle button 3182, an evacuation chamber port 3184, an evacuation chamber collar 3185, an inner shaft (catheter) handle 3190, an inner shaft (catheter) handle button 3192, a wire termination collar 3194, and a heated sheath plug 3196. In some embodiments, the outer shaft 3160, the expandable structure 3166, the inner shaft 3170, the nozzle 3174, and the expandable structure 3176 can be the same or substantially similar to the outer shaft 3060, the expandable structure 3066, the inner shaft 3070, the nozzle 3074, and the expandable structure 3076, as described above with reference to FIG. 33. Thus, certain aspects of the outer shaft 3160, the expandable structure 3166, the inner shaft 3170, the nozzle 3174, and the expandable structure 3176 are not described in greater detail herein.

In some embodiments, the user can push the outer shaft handle button 3182 to advance an outer liner or sheath 3161 of the outer shaft 3160 distally. Advancement of the outer liner 3161 relative to a tip 3163 of the outer shaft 3160 can cause the expandable structure 3166 to expand (e.g., transition into an expanded configuration), such that the expandable structure 3166 can hold the tip 3163 in position inside the gallbladder. In some embodiments, the outer shaft handle 3180 can include a locking mechanism (not shown), such that the liner 3161 of the outer shaft 3160 can lock into position relative to the tip 3163. The evacuation chamber port 3184 is in fluidic communication with the interior of the outer shaft 3160. Cryoablation medium can flow through the interior of the outer shaft 3160 and exit the ablation system 3150 via the evacuation chamber port 3184. In some embodiments, the evacuation chamber port 3184 can be connected to a hose and/or a vacuum line, such that cryoablation medium can be evacuated from the outer shaft 3160 and the handle assembly 3180 on demand.

The evacuation chamber collar 3185 fits around the outside of the inner shaft 3170. In some embodiments, the evacuation chamber collar 3185 can create a seal with the inner shaft 3170, such that the evacuation chamber collar 3185 can prevent liquid and/or gas (e.g., of the ablation medium) from leaking or flowing further along the inner shaft 3170.

The inner shaft handle 3190 includes an inner shaft handle button 3192. Pressing the inner shaft handle button 3192 can advance a portion of the inner shaft 3170 relative to the inner handle assembly 3190. In some embodiments, pressing the inner shaft handle button 3192 can advance one or more outer layers of the inner shaft 3170 relative to an inner ablation lumen of the inner shaft 3170. In some embodiments, the inner handle assembly 3190 can include a locking mechanism (not shown), such that the portion of the inner handle assembly 3180 that has been advanced can lock into position relative to the other portions of the inner shaft 3170. This movement of the portion of the inner shaft 3170 can be used to deploy the expandable structure 3176.

The wire termination collar 3194 couples to the inner shaft 3170 and can serve as a connection point between one or more heating elements, sensors, lumens, etc. and external sources. Alternatively, in some embodiments, the collar 3194 can be omitted and connections can be formed between one or more components of the inner shaft 3170 and external sources via another section of the handle 3190. In some embodiments, the collar 3194 can be configured to couple one or more heating wires of the inner shaft 3170 to an external heat source. In some embodiments, the wire termination collar 3194 can provide heat to the inner shaft 3170 via the internal heat source to prevent clogging due to freezing. Cryoablation medium can cause materials passing through the inner shaft 3170 and/or outer shaft 3160 to freeze, thereby clogging the pathway through the inner shaft 3170. By activating heating (e.g., via the external heat source coupled to one or more heating wires that extend along the inner shaft 3170), the heat applied to the inner shaft 3170 can melt frozen materials, allowing flow through the inner shaft 3170 and/or outer shaft 3160. In some embodiments, the wire termination collar 3194 can be coupled to the inner handle assembly 3190. The collar 3194 can include a heated sheath plug 3196 that is used to couple to an external heat source.

While not shown in detail in FIGS. 34A and 34B, the inner shaft 3170 defines a lumen that can deliver an ablation medium, such as, for example, cryoablation medium, to openings of the nozzle 3174. In some embodiments, the nozzle 3174 can be rotatable to adjust the location of the openings of the nozzle 3174 and where the ablation medium is being delivered. In some embodiments, the nozzle 3174 can actuate independently of the expandable structure 3176, e.g., be moved relative to the expandable structure 3174, to adjust the locations of the openings of the nozzle 3174 and where the ablation medium is being delivered.

In some embodiments, a pressure sensing lumen (not shown) can be disposed in one or more of the outer shaft 3160 and/or the inner shaft 3170. In some embodiments, the pressure sensing lumen can be fluidically coupled to a pressure sensor at a proximal end of the ablation system 3150 (not shown). In some embodiments, the pressure sensing lumen can terminate at an orifice that is disposed in the gallbladder cavity, while the pressure sensor is located outside of the gallbladder cavity. In other words, the pressure sensing lumen can fluidically couple an interior of the gallbladder cavity to the pressure sensor. In some embodiments, the pressure sensing lumen can be disposed about the inner shaft 3170. In some embodiments, the pressure sensing lumen can be disposed about the outer shaft 3160. In some embodiments, the pressure sensing lumen can be disposed in the inner shaft 3170. In some embodiments, the ablation system 3150 can include multiple pressure sensing lumens.

FIG. 34B shows spray patterns of the ablation system 3150, according to an embodiment. As described above, the ablation system 3150 can be used to deliver an ablation medium, such as a cryoablation medium, via openings of the nozzle 3174. In some embodiments, the medium can be delivered as a fluid. In some embodiments, the medium can be delivered as a gas. In some embodiments, the medium can be delivered as a fluid that transitions into a gas at a point along the length of the ablation system 3150 and/or within the gallbladder lumen. As shown schematically in FIG. 34B, the spray pattern of the ablation medium exiting the ablating catheter 3150 via the openings of the nozzle 3174 can be conical. In other words, the ablation medium can be delivered via multiple spray zones that may or may not overlap with one another.

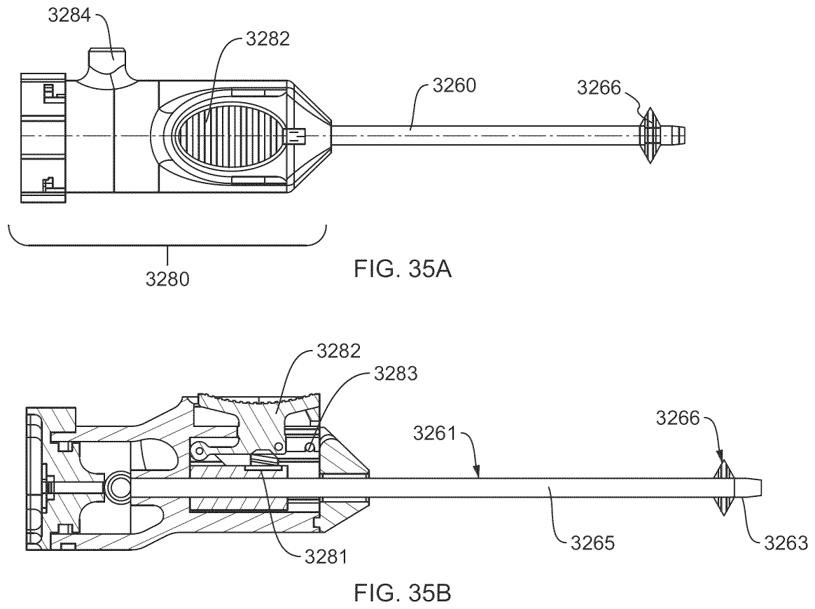

FIGS. 35A-35B provide more detailed views of an outer shaft 3260 and an outer shaft handle 3280 of an ablation system, according to an embodiment. FIG. 35A depicts a side view of the outer shaft 3260 and the outer shaft handle 3280, and FIG. 35B depicts a cross-sectional view of the outer shaft 3260 and the outer shaft handle 3280. As shown, the outer shaft 3260 includes an expandable structure 3266 (e.g., retention mechanism). The outer shaft 3260 is coupled to an outer shaft handle 3280. The outer shaft handle 3280 includes an outer shaft handle button 3282 and an evacuation chamber port 3284. In some embodiments, the outer shaft 3260, the expandable structure 3266, the outer shaft handle 3280, the outer shaft handle button 3282, and the evacuation chamber port 3284 can be the same or substantially similar to the outer shaft 3160, the expandable structure 3166, the outer shaft handle 3180, the outer shaft handle button 3182, and the evacuation chamber port 3184, as described above with reference to FIGS. 34A-34B. Thus, certain aspects of the outer shaft 3260, the expandable structure 3266, the outer shaft handle 3280, the outer shaft handle button 3282, and the evacuation chamber port 3284 are not described in greater detail herein. The outer shaft 3260 can define a lumen 3265 that can receive an inner shaft (e.g., an inner shaft of an ablation catheter, such as any of those described herein).

The handle 3280 can have a button 3282 that can be moved (e.g., slid) distally to advance an outer liner 3261 of the outer shaft 3260 relative to a tip 3263 of the outer shaft 3260. This advancement can be used to deploy the expandable structure 3266, e.g., transition the expandable structure 3266 from a collapsed state where it extends generally parallel to a longitudinal axis of the outer shaft 3260 to an expanded state where it bows radially outwards from the longitudinal axis. The expandable structure 3266 once deployed can be configured to retain the distal end of the outer shaft 3260 within an gallbladder lumen. In other words, the expandable structure 3266 can be configured to have a diameter in its expanded state that is larger than an opening through which the distal end of the outer shaft 3260 has used to gain access to the gallbladder lumen. As such, the expanded structure 3266 in its expanded state can rest against the walls of the gallbladder near that opening to retain the distal end of the outer shaft 3260 within the gallbladder lumen. The button 3282 can be locked by a spring 3281. The button 3282 can be depressed to unlock the button 3282 and then slid to advance the liner 3161. Once the button has slid its maximum distance (e.g., along a track), the button 3282 can be locked once again via a notch 3283 and the spring 3282 that presses the button 3282 into the notch. While a button is described as an example of an actuator (e.g., actuator 801a), it can be appreciated that any type of actuation mechanism can be used to advance and/or retract various components of the outer shaft 3260.

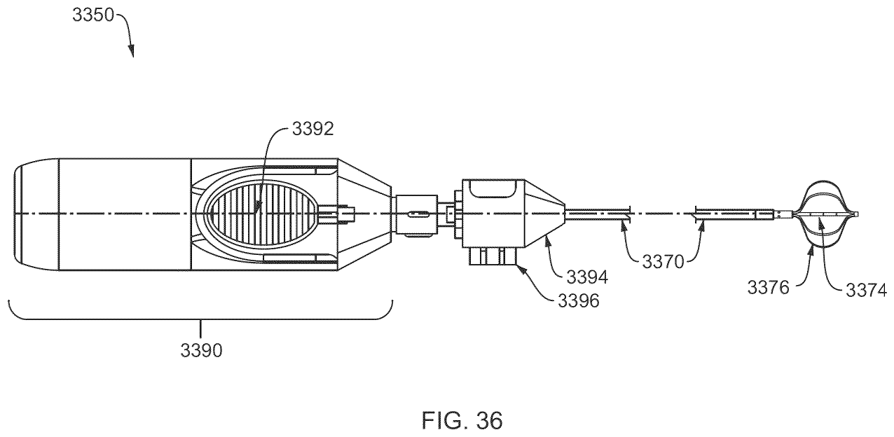

FIG. 36 provides a more detailed view of an ablation catheter 3350 of an ablation system, according to an embodiment. As shown, the ablation catheter 3350 includes an inner shaft 3370, a nozzle 3374, an expandable structure 3376 (e.g., expandable cage), an inner shaft handle 3390 with an inner shaft handle button 3392, a wire termination collar 3394, and an inner shaft plug 3396. In some embodiments, the inner shaft 3370, the nozzle 3374, the expandable structure 3376, the inner shaft handle 3390, and the inner shaft handle button 3392, the wire termination collar 3394, and the inner shaft plug 3396 can be the same or substantially similar to the inner shaft 3170, the nozzle 3174, the expandable structure 3176, the inner shaft handle 3190, the inner shaft handle button 3192, the wire termination collar 3194, and the inner shaft plug 3196, as described above with reference to FIGS. 34A-34B. Thus, certain aspects of the inner shaft 3370, the nozzle 3374, the expandable structure 3376, the inner shaft handle 3390, the inner shaft handle button 3392, the wire termination collar 3394, and the inner shaft plug 3396 are not described in greater detail herein.

In some embodiments, the distal end of the ablation catheter 3350 can be inserted through a lumen of an outer shaft or introducer, e.g., as depicted in FIG. 34A. For illustrative purposes in FIG. 36, the inner shaft 3370 is shown with a discontinuity to indicate that a length of the inner shaft is longer than that shown in FIG. 36. The expandable structure 3376 can be transitioned between an undeployed configuration and a deployed configuration. When the expandable structure 3376 is in the undeployed configuration, the expandable structure 3376 can have elongate members that extend substantially parallel to a longitudinal axis of the catheter 3350 and, in particular, a longitudinal axis of the shaft 3370. In such configuration, the distal portion of the shaft 3370 (including the nozzle 3374 and the expandable structure 3376) can be inserted through a lumen of an outer shaft or introducer, e.g., into a gallbladder lumen. After the distal portion of the shaft 3370 has been inserted past a distal end of the outer shaft, then the expandable structure 3376 can be transitioned into the deployed configuration, where the elongate members of the expandable structure 3376 extend outward (e.g. bow out radially) from the longitudinal axis.

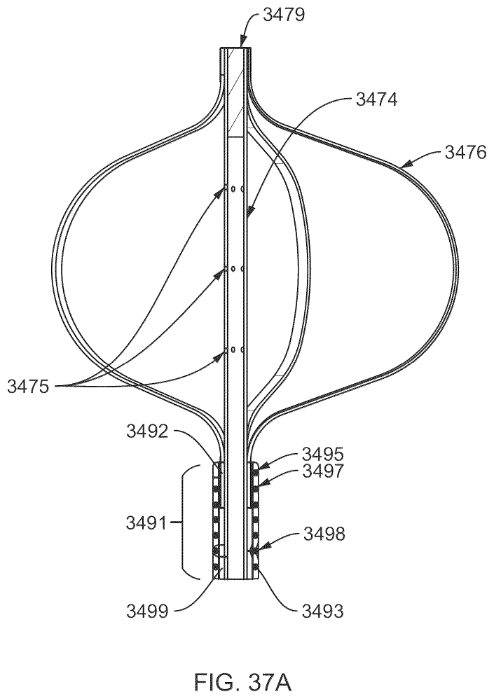

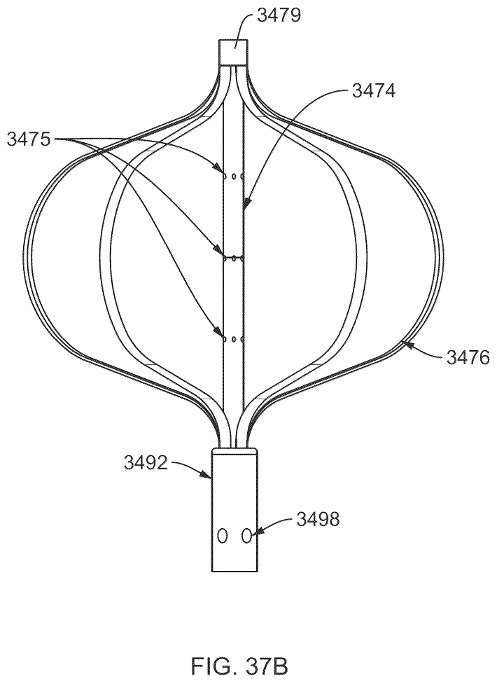

FIGS. 37A-37B are detailed viewed of a distal portion of an ablation catheter with heating elements, according to an embodiment. FIG. 37A shows a cross-sectional view of the distal portion, with portions of the interior shown, while FIG. 37B is an exterior view of the distal portion. As shown, the distal portion of the ablation catheter includes a nozzle 3474 including a plurality of nozzle openings or fenestrations 3475, an expandable structure 3476 (e.g., expandable cage), a hub 3479, and a heated sheath 3491. The heated sheath 3491 includes a jacket 3492, a liner 3493, a sensor wire or lead implemented as a thermocouple wire 3495, a heating element implemented as a heating wire 3497, and vent openings or holes 3498. In some embodiments, the fenestrations 3475 can be the same or substantially similar to the fenestrations 1175, as described above with reference to FIG. 11, or other nozzle openings described herein. In some embodiments, the expandable structure 3476 can be the same or substantially similar to other expandable structures described herein, including, for example, expandable structure 876, as described above with reference to FIG. 9, and/or expandable structure 3176, as described above with reference to FIG. 34A. Thus, certain aspects of the fenestrations 3475 and the expandable structure 3476 are not described in greater detail herein.

The expandable structure 3476 can be formed of a plurality of elongate members. The plurality of elongate members are transitionable between an undeployed configuration in which the elongate members extend substantially parallel to a longitudinal axis of the catheter and a deployed configuration in which the elongate members extend outward (e.g., bow out radially) from the longitudinal axis. In some embodiments, each of the elongate members can have a proximal end that is coupled to a distal end of the heated sheath 3491 and a distal end that is coupled to a hub 3479. In such embodiments, deployment of the elongate members can be made by moving the hub 3479 or the heated sheath 3491 relative to the other of the hub 3479 and the heated sheath 3491. For example, the heated sheath 3491 can be advanced distally toward the hub to cause the elongate members to extend outward (e.g., bow out radially) and to deploy the expandable structure 3476.

The jacket 3492 and the liner 3493 of the heated sheath 3491 insulate the thermocouple wire 3495 and the heating wire 3497. In some embodiments, the jacket 3492 and/or the liner 3493 can be extruded over the wires 3495, 3497. The jacket 3492 is positioned exterior to the thermocouple wire 3495 and the heating wire 3497, while the liner 3493 is positioned interior to the thermocouple wire 3495 and the heating wire 3497. In some embodiments, the thermocouple wire 3495 and the heating wire 3497 can be wound together. In some embodiments, the thermocouple wire 3495 can be laid straight under the heating wire 3497.

The vent holes 3498 are configured to communicatively couple a pressure sensing lumen 3499 with an exterior of the catheter. As such, the vent holes 3498 can be configured to couple the pressure sensing lumen 3499 with a lumen of a gallbladder such that an intraluminal pressure of the gallbladder can be measured via the pressure sensing lumen 3499. The pressure sensing lumen 3499 can be an annular space that is disposed between an inner shaft defining a lumen for delivering the ablation medium and the heated sheath 3491. As shown, the catheter includes two vent holes 3498. Inclusion of multiple vent holes 3498 can allow the maintain coupling between the pressure sensing lumen 3499 and a body lumen when one of the vent holes is clogged. In some embodiments, the catheter can include 3, 4, 5, 6, 7, 8, 9, 10, or more than about 10 vent holes 3498.

The thermocouple wire 3495 can be configured to couple a temperature sensor (e.g., thermocouple) with a control unit or processor (e.g., control unit 110) at a proximal end of the ablation catheter (or operatively coupled to a proximal end of the ablation catheter). The temperature sensor can be disposed near the vent holes 3498 and/or outside of the ablation catheter to measure a temperature near the distal portion of the ablation catheter.

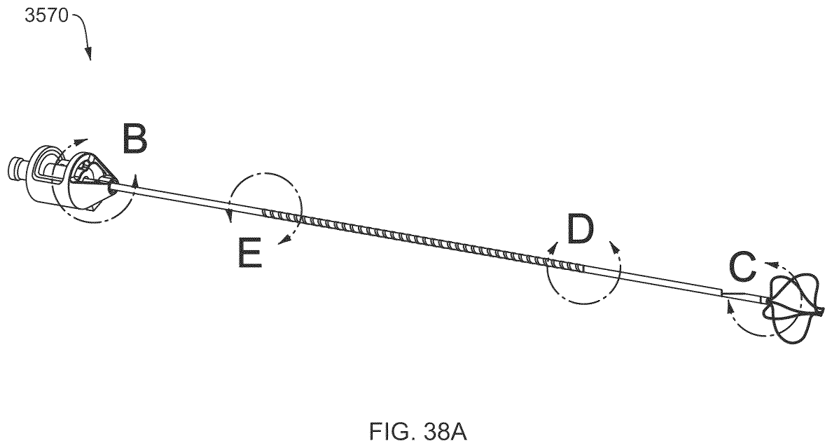

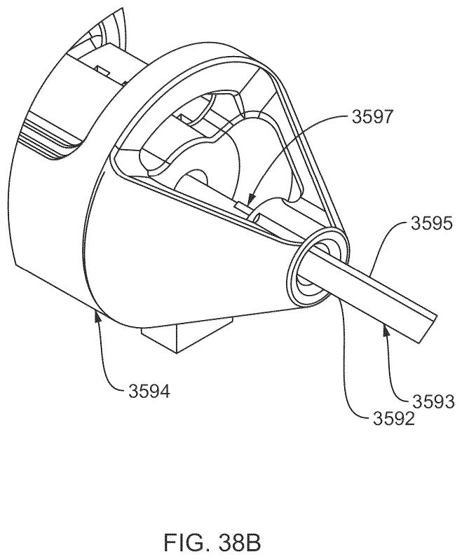

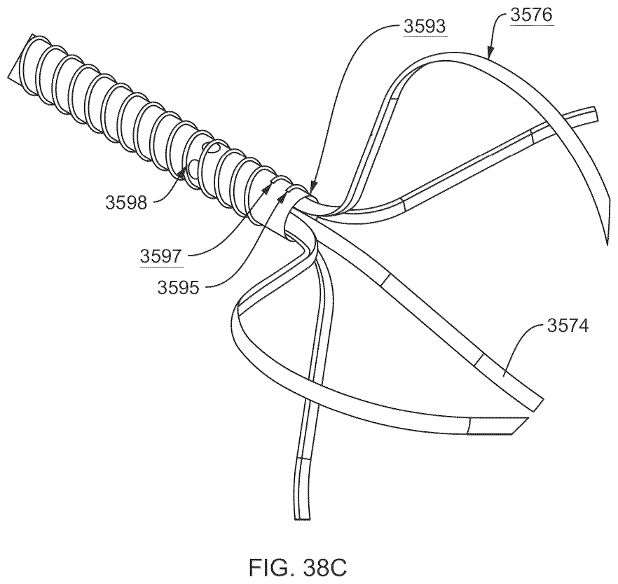

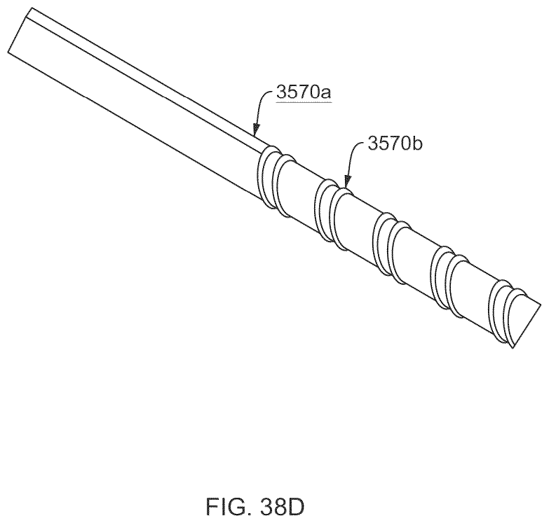

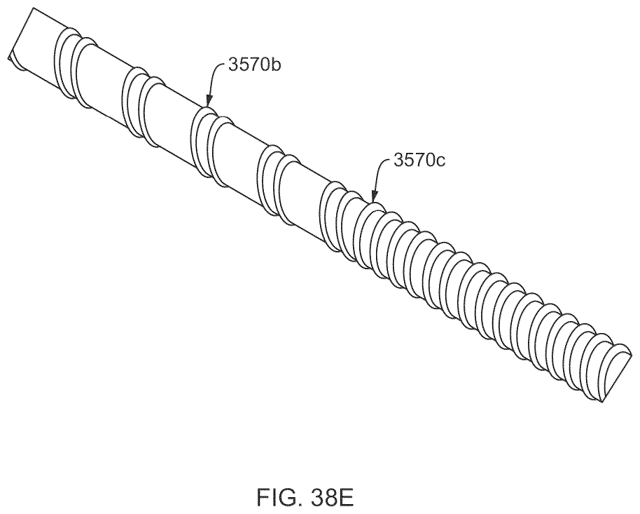

FIGS. 38A-38E show an ablation catheter assembly 3570, and details thereof, according to an embodiment. FIG. 38A shows the full catheter assembly 3570, while FIG. 38B shows details of section B, as marked in FIG. 38A, FIG. 38C shows details of section C, as marked in FIG. 38A, FIG. 38D shows details of section D, as marked in FIG. 38A, and FIG. 38E shows details of section E, as marked in FIG. 38E. As shown, the catheter assembly 3570 includes a nozzle 3574, an expanded structure 3576, a jacket 3592, a liner 3593, a wire termination collar 3594, a thermocouple wire 3595, a heating wire 3597, and vent holes 3598. In some embodiments, the nozzle 3574, the expanded structure 3576, the jacket 3591, the liner 3593, the thermocouple wire 3595, the heating wire 3597, the wire termination collar 3594, and the vent holes 3598 can be the same or substantially similar to like components described in other embodiments herein, including, for example, the nozzle 3174, the expandable structure 3176, the wire termination collar 3194, and the inner shaft plug 3196, as described above with reference to FIGS. 34A-34B and/or the nozzle 3474, the expanded structure 3476, the jacket 3492, the liner 3493, the thermocouple wire 3495, the heating wire 3497, and the vent holes 3498, as described above with reference to FIGS. 37A-37B. Thus, certain aspects of the nozzle 3574, the expanded structure 3576, the jacket 3591, the liner 3593, the wire termination collar 3594, the thermocouple wire 3595, the heating wire 3597, and the vent holes 3598 are not described in greater detail herein. The heating wire 3597 and/or thermocouple wire 3595 can be wound around portions of the sheath, the shaft, and/or an annular space between the sheath and the shaft to heat those portions.

In some embodiments, the thermocouple wire 3595 and/or the heating wire 3597 can couple to one or more connections in the wire termination collar 3595. For example, the thermocouple wire 3595 can be configured to couple via the collar 3595 to an external processor or control unit (e.g., control unit 110), e.g., for monitoring temperature, pressure, and/or other conditions and/or controlling the delivery and/or evacuation of the ablation medium. The heating wire 3597 can be configured to couple to an external heat source via the collar 3595, e.g., for receiving energy from the external heat source and to generate heat for heating portions of the ablation catheter. In some embodiments, the wire termination collar 3595 can be coupled to a proximal handle for operating the ablation catheter, as described with reference to FIG. 34A. In some embodiments, the collar 3595 can be omitted, and the thermocouple wire 3595 and/or the heating wire 3597 can be configured to couple to the handle (including any onboard components, such as, for example, an onboard processor and/or microcontroller, a power source, etc.).

As shown in FIGS. 38D and 38E, the thermocouple wire 3595 and the heating wire 3597 are divided into a straight section 3570a, a coarse wound section 3570b, and a finely wound section 3570c. A tighter pitch of the heating wire 3597 (i.e., a finer wound heating wire) can increase the energy density of a particular region. In some embodiments, the finely wound section 3570 c can have a length of about 0.5 cm, about 1 cm, about 1.5 cm, about 2 cm, about 2.5 cm, about 3 cm, about 3.5 cm, about 4 cm, about 4.5 cm, or about 5 cm, inclusive of all values and ranges therebetween. In some embodiments, the finely wound section 3570 c can cover about 5%, about 10%, about 15%, about 20%, about 25%, or about 30% of the full length of the full catheter assembly 3570, inclusive of all values and ranges therebetween. In some embodiments, the transition from the coarse wound section 3570b to the finely wound section 3570c can be gradual, or the spacing between the thermocouple wire 3595 and the heating wire 3597 can change as a gradient. In some embodiments, the transition from the coarse wound section 3570b to the finely wound section 3570c can be immediate.

In some embodiments, adjacent turns of the wires can be spaced apart in the coarse wound section by about 0.5 mm, about 1 mm, about 1.5 mm, about 2 mm, about 2.5 mm, about 3 mm, about 3.5 mm, about 4 mm, about 4.5 mm, or about 5 mm, inclusive of all values and ranges therebetween. In some embodiments, the wires can be spaced apart in the finely wound section 3570 c by about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, or about 2 mm, inclusive of all values and ranges therebetween.

Power delivered to the thermocouple wire 3595 and the heating wire 3597 can be a function of operating voltage and wire resistance. Operating voltage and wire resistance can be tuned to achieve a desired energy density through the cross-sectional area of the heating wire, thus determining the thermal flux generated by the heating wire 3597 and the temperature response to cooling.

While a single heating element is depicted in FIGS. 38A-38A, it can be appreciated that any number of heating elements can be used. For example, multiple heating wires that extend along different portions of the ablation catheter (e.g., different portions of a sheath, a shaft, or annular space therebetween). In some embodiments, multiple heating wires can be selectively activated, e.g., using a processor (e.g., control unit 110), to maintain substantially uniform temperature along an entire length of the sheath, the shaft, or the annular space therebetween. Substantially uniform temperature can be, for example, temperatures along the entire length that do not deviate more than 10% from an average or median temperature. In some embodiments, one or more sensors (e.g., coupled to thermocouple wires) can be disposed at one or more locations along the length of the shaft and used to measure temperatures at different points along the length of the shaft. These measured temperatures can be received at the processor and used to control the one or more heating elements (e.g., selectively activate or adjust amount of power being delivered to the heating elements).

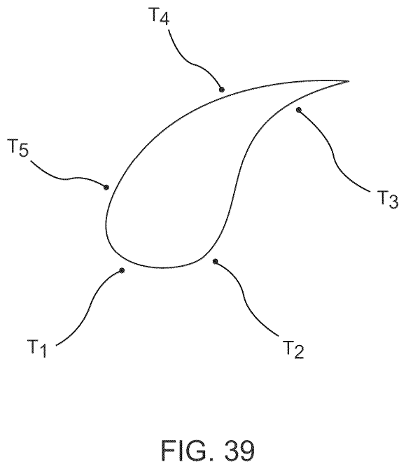

In some embodiments, the thermocouple wire 3595 can be coupled to a temperature sensor disposed near a distal end of the ablation catheter. The thermocouple wire 3595 can carry the temperature signal to a processor (e.g., an onboard processor and/or external processor) for monitoring of temperature and/or control of ablation delivery and/or evacuation based on temperature. In some embodiments, ablation devices and/or systems described herein can be used with external temperature probes. FIG. 39 is a depiction of placement of temperature sensors (e.g., probes or needle-like temperature sensors) throughout a length of a gallbladder, according to an embodiment. As shown, the gallbladder includes a neck region, a body region, and a fundus region. Temperature probes can be placed outside of the wall of the gallbladder to monitor how temperature changes along the length of the gallbladder, e.g., from neck through fundus. The placement of temperature probes can confirm that temperature changes have pervaded from the inside to the outside of the gallbladder, which can facilitate determination of the efficacy of the ablation. In other words, the temperature probes can also aid in confirming that cryoablation has occurred, i.e., the gallbladder wall has been ablated. In some embodiments, a first temperature probe T1 can be placed at a distal end of the gallbladder. Measuring the temperature at the fundus end of the gallbladder can confirm that the cryoablation medium has penetrated to that end of the gallbladder. In some embodiments, a temperature probe can be placed at a point between the fundus end and the neck of the gallbladder (e.g., temperature probe T2). In some embodiments, a temperature probe can be placed in the neck region near the opening of the gallbladder into the cystic duct (e.g., temperature probe T3). In some embodiments, an additional temperature probe can be placed in the neck region of the gallbladder (e.g., temperature probe T4). In some embodiments, an additional temperature probe can be placed at a point between the fundus and the neck of the gallbladder (e.g., temperature probe T5). In some embodiments, a combination of at least three temperature sensors (e.g., one placed at neck, one placed at body, one placed at fundus) can be used to measure the temperature of the tissue wall along a length of the gallbladder. In some embodiments, depending on the temperature measurements, a location of the nozzles of the ablation catheter can be adjusted, e.g., translated distally and/or proximally to target regions of tissue having higher temperatures. As such, the placement of the temperature probes can be used to confirm even distribution of ablation medium within the gallbladder and/or provide feedback for controlling further delivery of ablation medium.

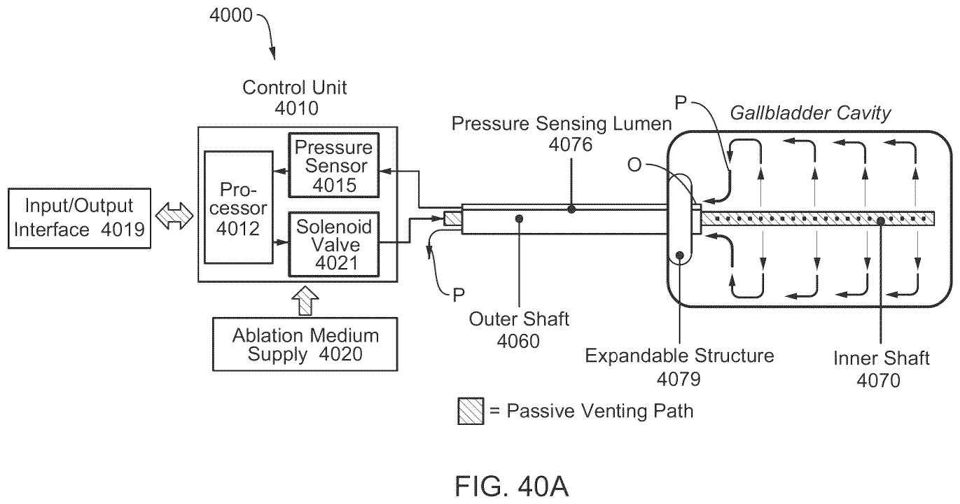

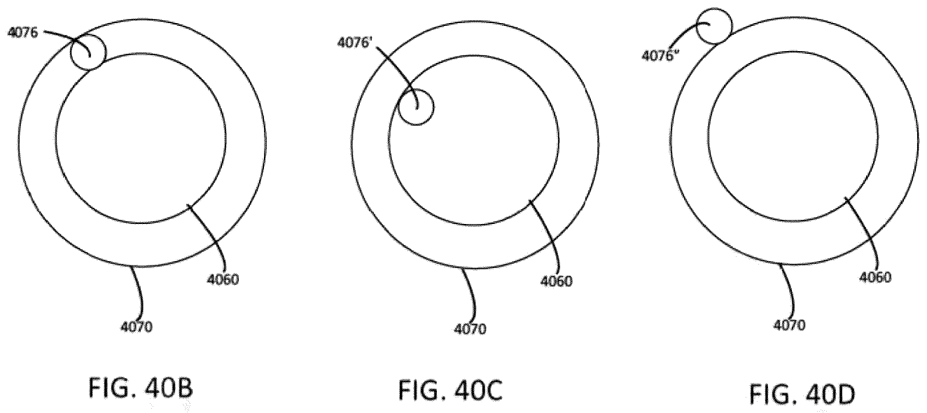

FIGS. 40A-40D are illustrations of ablation system implemented as a cryoablation device 4000. The cryoablation device 4000 can be configured to ablate or defunctionalize a gallbladder cavity. The cryoablation device 4000 can include components that are structurally and/or functionally similar to other ablation systems and components thereof described herein. FIG. 40A shows a side profile view of the cryoablation device 4000, while FIGS. 40B-40D show cross sectional views of various configurations of lumens of inner and outer shafts of the cryoablation device 4000. The cryoablation device 4000 includes a control unit 4010, an outer shaft 4060, an inner shaft 4070, and a pressure sensing lumen 4076. The control unit 4010 includes a processor 4012, a pressure sensor 4015, an input/output interface 4019, and a solenoid valve 4021. In some embodiments, an expandable structure 4079 can hold the inner shaft 4070 in the gallbladder cavity during deployment of an ablation medium. The control unit 4010 is fluidically coupled to an ablation medium supply 4020. In some embodiments, ablation medium can flow from the ablation medium supply to the inner shaft 4070, passing by the solenoid valve 4021.

In some embodiments, the pressure sensing lumen 4076 can terminate at an orifice O that is disposed in the gallbladder cavity, while the pressure sensor 4015 is located outside of the gallbladder cavity. In other words, the pressure sensing lumen 4076 can fluidically couple an interior of the gallbladder cavity to the pressure sensor 4015. The pressure sensor 4015 can be disposed at the control unit 4010 and/or operatively coupled to the control unit 4010. In such a configuration, the pressure sensor 4015 can measure the pressure inside of the gallbladder cavity while being positioned outside of the gallbladder cavity.

The pressure sensing lumen 4076 can be disposed about the inner shaft 4070 or outer shaft 4060 of the catheter system according to one of several different arrangements. In some embodiments, the pressure sensing lumen 4076 can have a circular cross-section and be disposed to one side of a shaft. For example, in an embodiment, the pressure sensing lumen 4076 can be affixed to the inner shaft 4070, as shown in FIG. 40B. Alternatively, a pressure sensing lumen 4076′ can be disposed or defined within the inner shaft 4070, as shown in FIG. 40C. As yet another alternative, a pressure sensing lumen 4076″ can be disposed outside the outer shaft 4060, as shown in FIG. 40D. As additional alternatives, a pressure sensing lumen can be integrated into a wall of the inner or outer shaft, coupled to both inner and outer shafts, etc. In some embodiments, a pressure sensing lumen 4076 can be an annular space that is formed between outer and inner concentric sheaths and/or shafts of the inner shaft 4070, such as described with reference to FIG. 37A. Pressure can be relieved from the gallbladder cavity via venting paths P that pass through the outer shaft 4060 and to the outside of the cryoablation device 4000. The pressure sensing lumen may also be referred to as a pressure lumen or a sensor lumen.

While not expressly identified in FIGS. 40A-40D, the cryoablation device 4000 includes a lumen defined by inner and outer shafts 4060, 4070 for delivery of an ablation medium and/or evacuation of an ablation medium from the gallbladder cavity. For example, similar to other catheter systems described herein, the inner shaft 4070 can define a lumen for delivery of a cryogenic ablation medium to the gallbladder cavity, e.g., via one or more nozzle openings. The outer shaft 4060 can define a for evacuating the cryogenic ablation medium from the gallbladder cavity.

Systems, devices, and methods described herein can implement a passive evacuation channel and cryogen control system to safely vent cryogen gas from the gallbladder cavity, while ensuring safe operating conditions. During cryogen delivery, a cryogenic ablation medium (e.g., nitrous oxide) expands and evacuates to an external environment (e.g., atmosphere) through an annular space between an inside surface of the outer shaft 4060 and an outer surface of the inner shaft 4070. Resistance in the evacuation channel can cause the gallbladder cavity to distend, to facilitate exposure of tissue within the lumen to the cryogenic ablation medium. The solenoid valve 4021 can be configured to control or regulate delivery of the ablation medium, e.g., from the ablation medium supply 4020, into the gallbladder lumen. For example, the control unit 4010 can control the solenoid valve 4021 to transition from an open state in which ablation medium can be delivered into the gallbladder lumen to a closed state in which ablation medium can be prevented from being delivered into the gallbladder lumen. While a solenoid valve is provided as the example valve herein, it can be appreciated that other types of valves, including mechanically actuated valves, magnetically actuated valves, etc. can be used to control the delivery of the ablation medium into the gallbladder lumen. The control unit 4010, pressure sensor 4015, and solenoid valve 4021 can produce a closed-loop pressure feedback system for maintaining safe operating pressures within the gallbladder cavity. In particular, in response to detecting a pressure within the gallbladder cavity that is greater than a predetermined maximum threshold, the control unit 4010 can control the solenoid valve 4021 to terminate supply of the ablation medium into the gallbladder cavity and/or evacuate via the outer shaft 4060 the ablation medium from the gallbladder cavity to an external environment. Additionally or alternatively, in response to detecting a pressure within the gallbladder cavity that is less than a predetermined minimum threshold, the control unit 4010 can control the ablation medium supply 4020 and/or solenoid valve 4021 to provide additional ablation medium into the gallbladder cavity to sufficiently distend the gallbladder for cryoablation.

When cryoablating a body cavity such as the gallbladder, high relative humidity within the cavity can lead to freezing of the moisture present within the internal organ during cryoablation and result in formation of ice particles or other solids. These ice particles or other solids can clog an evacuation pathway of a catheter assembly that is being used to ablate the internal organ. With a cryoablation medium, where the phase translation of the medium from a liquid to a gas can bring about high volumetric changes and therefore pressure buildup absent a functional evacuation pathway, clogging of the evacuation pathway can lead to undesirable high pressures within the cavity and potential injury to a patient. As previously described (e.g., with reference to ablation catheter assembly 3570), one approach to prevent blockade of such catheters is to heat portions of the catheter assembly so as to prevent freezing within the evacuation pathway.

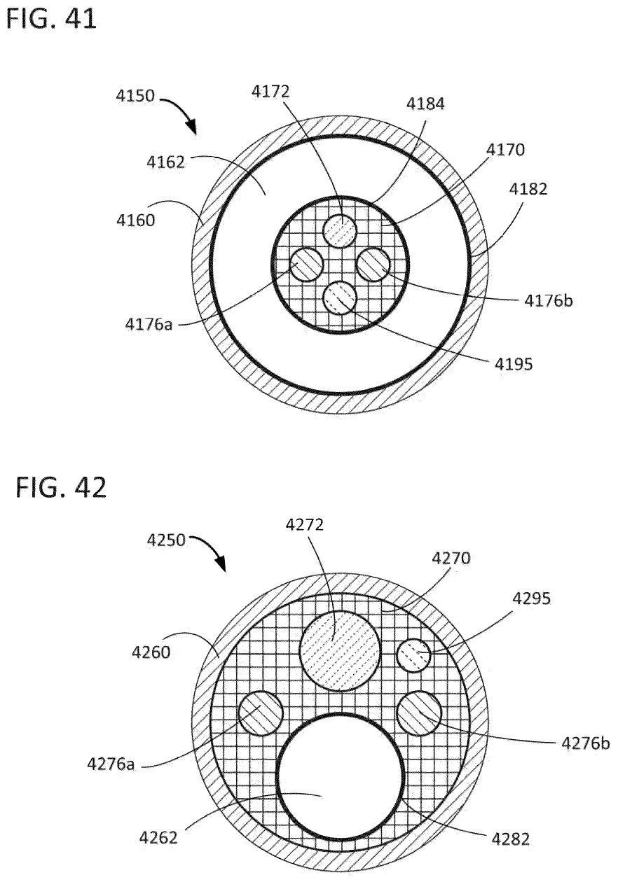

For example, FIG. 41 is an illustration of an ablation system 4150, according to an embodiment. The ablation system 4150 includes one or more heating elements configured to combat ice build-up in an evacuation lumen of the ablation system 4150. As shown, the ablation system 4150 includes an outer shaft 4160 and an inner shaft 4170 with a space between the two shafts defining an evacuation pathway 4162. The inner shaft 4170 can be disposed concentrically or co-axially with the outer shaft 4160. In some embodiments, the outer shaft 4160 can be an access sheath, and the inner shaft 4170 can be the shaft or elongate body of an ablation catheter.

The inner shaft 4170 defines a first lumen 4172 configured to deliver a cryogenic ablation medium to the internal volume or cavity, for example, the gallbladder. The inner shaft 4170 can also define a pair of pressure sensing lumens 4176a and 4176b that can be in fluidic communication with pressure sensors, or have a pressure sensor disposed therein. The inner shaft 4170 can also define a temperature sensing lumen 4195 that can be in thermal communication with a temperature sensor, or have a temperature sensor disposed therein, which is configured to measure a temperature of the internal volume or cavity defined by the internal organ.

During cryoablation of the internal volume or cavity, the evacuation pathway 4162 can be configured to evacuate gases or other fluid from the cavity. In some embodiments, the evacuation via the evacuation pathway 4162 can be passive, e.g., driven by a pressure differential between atmosphere and the cavity. In some embodiments, a negative pressure or vacuum may be applied to the evacuation pathways 4162 to aspirate the internal volume. As previously described, introduction of the cryogenic ablation medium into the internal volume can cause moisture to freeze and form ice particles that can block or clog the evacuation pathway 4162 leading to deterioration in evacuation performance, failure of the ablation system 4150, and/or dangerous pressure buildup within the cavity.

To inhibit clogging of the ablation system 4150, the outer shaft 4160 can include a first heating element 4182 disposed on an inner radial surface of the outer shaft 4160 and the inner shaft 4170 can include a second heating element 4184 disposed on an outer radial surface of the inner shaft 4170. The heating elements 4182, 4184 can include heating coils formed of a resistive heating element, such as, for example, a resistive wire, that transfers electrical energy into heat, thereby conductively heating nearby bodies. In some embodiments, the first heating element 4182 is disposed or wrapped around the inner circumference of the outer shaft 4160, but in other embodiments, may be disposed or wrapped around an outer circumference of the outer shaft 4160. In some embodiments, the first heating element 4182 is embedded within the outer shaft 4160 wall material. Similarly, the second heating element 4184 may be disposed or wrapped around an outer circumference of the inner shaft 4170 or embedded within the inner shaft 4170 (e.g., proximate to the outer radial surface of the inner shaft 4170).

In some embodiments, the first heating element 4182 and/or the second heating element 4184 may include resistive heating wires wrapped in a helical coil configuration with about 0 inch to about 1 inch pitch spacing, including all subranges and values in between. In some embodiments, the resistive heating wires are wrapped in a helical coil configuration with a fixed pitch. In some embodiments, the resistive heating wire is wrapped in a helical coil configuration with a variable or “progressive fix” such that the sections of tighter coil pitch are located closer to the distal end of the outer shaft 4160 and/or the inner shaft 4170. As described above, such progressively closer wounding may be necessary to account for lower temperatures at or near a distal end of the ablation system 4150.

In some embodiments, the first heating element 4182 and/or the second heating element (e.g., resistance wires) can be configured to deliver heat energy at an energy density in a range of about 0.1 W/cm² to about 1.0 W/cm² (e.g., about 0.1, 0.2, 0.3, 0.4, 0.5, 0.6, 0.7, 0.8, 0.9, or 1.0W/cm², inclusive of all ranges and values therebetween).

In some embodiments, the heating elements 4182, 4184 can include a liquid medium with a high heat capacity (e.g., water) that can be circulated through contained channels on an ablation catheter 4150 and/or an introducer (not shown) to prevent fluidic pathways from freezing. In addition, the fluid can be heated before circulation to improve the freezing resistance of the fluidic pathways.

In some embodiments, the temperature sensor (e.g., a thermocouple) that is in thermal communication with the temperature sensing lumen 4195 or disposed therewithin can be used to provide temperature feedback to a controller (e.g., the control unit 310) used to control operation of the heating elements 4182, 4184. The controller may be configured to dynamically control power generated by the heating elements 4182, 4184 in response to dynamic cryogenic ablation medium so as to maintain a temperature in the evacuation pathway 4162 and/or adjust the temperature to melt ice particles and/or inhibit ice particle formation.

The heated annular evacuation design as depicted in FIG. 41, however, is complex. If not implemented appropriately, the heating provided by such a setup may not be sufficiently concentrated or cover all portions of the evacuation pathway. Any unheated locations along the evacuation pathway may result in freezing. Given the annular positioning of the evacuation pathway, more energy may also be required to heat the pathway. As such, it may be desirable to have a multi-lumen evacuation design with a dedicated lumen for evacuation. Such a multi-lumen configuration may reduce complexity in manufacturing the heated evacuation pathway 4162, thus reducing manufacturing costs.

FIG. 42 is an illustration of an ablation system 4250 with a multi-lumen evacuation design including a heated evacuation pathway 4262, according to an embodiment. The ablation system 4250 includes an outer shaft 4260, and an inner shaft 4270 disposed within a lumen defined by the outer shaft 4260. In some embodiments, an outer diameter of the inner shaft 4270 may be substantially equal to or in close tolerance with an inner diameter of the outer shaft 4260 such that an outer radial surface of the inner shaft 4270 is proximate to or substantially flush with the inner radial surface of the outer shaft 4260. In some embodiments, the outer shaft 4260 can be an access sheath, and the inner shaft 4270 can be the shaft or elongate body of an ablation catheter. Optionally, in some embodiments, a multi-lumen ablation catheter having a shaft 4270 can be used without an access sheath.

The inner shaft 4270 defines a first lumen 4272 configured to deliver a cryogenic ablation medium to the internal volume of an internal organ, for example, the gallbladder. The inner shaft 4270 can also define a pair of pressure sensing lumens 4276a and 4276b that can be in fluidic communication with pressure sensors, or have a pressure sensor disposed therein. The inner shaft 4270 can also define a temperature sensing lumen 4295 that can be in thermal communication with a temperature sensor, or have a temperature sensor disposed therein, which is configured to measure a temperature of the internal volume or cavity defined by the internal organ.

Different form the ablation catheter 4150, the inner shaft 4270 of the ablation catheter 4250 also defines an evacuation pathway 4262 configured to aspirate the internal volume of the internal organ. A heating element 4282 is disposed on, in, or about the evacuation pathway 4262 (e.g., an inner or an outer surface of the lumen that defines the evacuation pathway 4262). In some embodiments, the heating element 4282 may be embedded in the inner shaft 4260 proximate to the inner radial surface of the lumen that defines the evacuation pathway 4262. The heating element 4282 may be substantially similar to the heating elements 4182, 4184 and configured to heat the evacuation pathway 4262 to inhibit ice formation or melt ice particles that enter the evacuation pathway 4262 so as to inhibit clogging or blockage of the evacuation pathway 4262.

In some embodiments, the heating element 4282 can be a resistance wire wrapped around the lumen that defines the evacuation pathway 4262. In some embodiments, the heating element 4282 can be configured to deliver heat energy at an energy density in a range of about 0.1 W/cm² to about 1.0 W/cm² (e.g., about 0.1, 0.2, 0.3, 0.4, 0.5, 0.6, 0.7, 0.8, 0.9, or 1.0W/cm², inclusive of all ranges and values therebetween). In some embodiments, a temperature sensor (e.g., a thermocouple) can be used to provide temperature feedback to a controller (e.g., the control unit 310) used to control operation of the heating element 4282. In some embodiments, the temperature sensor can be is in thermal communication with the temperature sensing lumen 4295 or disposed therewithin. The controller may be configured to dynamically control power generated by the heating elements 4182, 4184 in response to dynamic cryogenic ablation medium so as to maintain a temperature in the evacuation pathway 4262 and/or adjust the temperature to melt ice particles and/or inhibit ice particle formation.

The multi-lumen construction depicted in FIG. 42 can simplify the heated evacuation construction, making it more commercially viable. In the multi-lumen construction, distinct lumens for evacuation (e.g., lumen defining evacuation pathway 4262), for cryogen delivery (e.g., lumen 4272), and for pressure sensing (e.g., lumens 4276a, 4276b) are used. In some embodiments, a separate aspiration catheter can be inserted through the evacuation pathway 4262 while the ablation system 4250 is deployed, e.g., to remove fluids and/or other substances from the body cavity and/or to actively evacuate the body cavity.

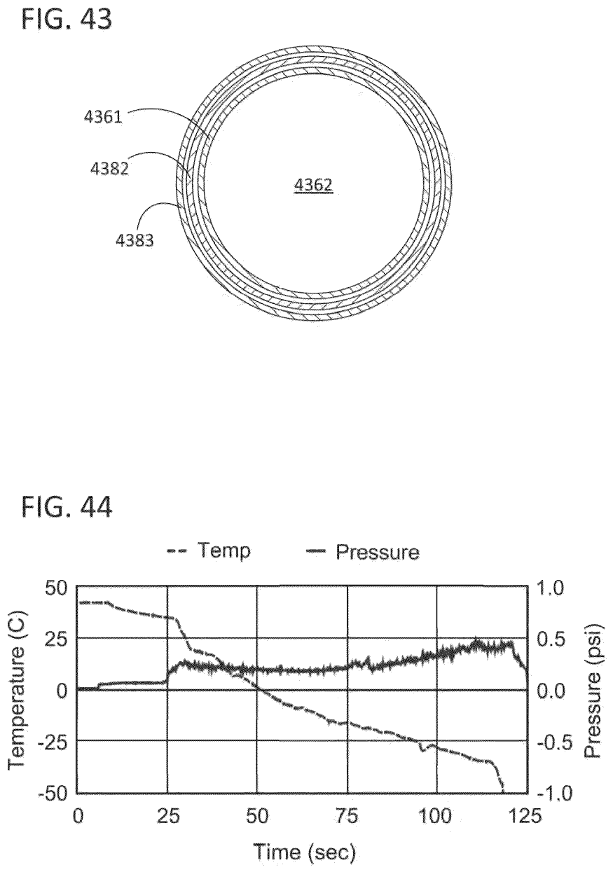

FIG. 43 is an illustration of a heated evacuation pathway 4362 that may be included in an ablation catheter, for example, the ablation catheter 4250, according to an embodiment. The evacuation pathway 4362 is configured to allow for evacuation of fluids or other substances from within an internal volume of an internal organ, as previously described. The evacuation pathway 4362 can have three layers. A first or innermost layer 4361 can be formed of a thermally conductive material (e.g., metals such as copper, alloys, etc.). A second layer can include a heating element 4382. The heating element 4382 may be substantially similar to the heating elements 4182, 4184 and configured to generate heat, thereby heating the evacuation pathway 4362. An outermost or insulating layer 4383 (e.g., an insulating polymer, fiberglass, mineral wool, glass wool, spray foam, polyvinyl chloride, melamine foam, cellulose, thermoplastic, polyurethane, silicone dioxide, phenol formaldehyde, any other suitable insulation material or any suitable combination thereof) can be disposed around the heating element 4382, and configured to inhibit heat transfer away from the evacuation pathway 4362. Moreover, the insulating layer 4383 may additionally inhibit unintended heating of surrounding components of the ablation catheter.

The heated evacuation pathway 4362 can be included in an ablation catheter, such as that depicted in FIG. 42. In some implementations, multiple heated evacuation pathways 4362 can be included in an ablation catheter to provide redundancy and achieve desired flow characteristics of the evacuated fluids. Having an inner lumen that is formed of a highly thermal conductive material (e.g., copper, stainless steel, aluminum, alloys, etc.) can allow efficient heat transfer from the heating element 4382 to the evacuation pathway 4362. The heating element 4382 may include a resistive heating wire wrapped around the inner lumen 4361 to achieve a desired energy density, and electrical power is applied to the resistive heating wire, converting electrical energy to heat. In some embodiments, the heating element 4382 may include a heating coil with a larger density or concentration of heating coils disposed near a tip of the inner lumen 4361 that is located within the internal organ (e.g., the gallbladder), wherein higher cryogen ablation medium loads may be experienced. In some embodiments, a temperature feedback loop may be used to maintain a temperature of the evacuation pathway 4362 at or about a predetermined temperature, for example, greater than about 0 degrees Celsius (e.g., greater than 0, greater than 1, greater than 1, greater than 2, greater than 3, or greater than 4 degrees Celsius, including all values and sub-ranges therebetween). In some embodiments, an embedded thermocouple in proximity to the heating element can be used to control lumen temperature in response to dynamic cryogen load. The thermocouple can be coupled to a proportional integral derivative (PID) controller, which can be configured to control the heating provided by the heating element 4382 based on temperature data captured by the thermocouple.

FIG. 44 is a plot of pressure and temperature of a cavity of a vapor load model used to simulate a body cavity undergoing a cryoablation process, according to embodiments. An ablation system including a heated evacuation lumen, such as, for example, ablation system 4250, was used to deliver a cryogenic ablation medium to the cavity of the vapor load model to perform the cryoablation. During the cryoablation process, the ablation system allowed fluid within the cavity to evacuate via a 16W heated evacuation pathway. The pressure and temperature of the cavity was then monitored over time, and shown in FIG. 44. As shown in FIG. 44, introduction of the cryogenic ablation medium causes a substantial drop in temperature in the cavity. As the cryoablation process continues, moisture in the internal volume can freeze to form ice particles that can become trapped in the evacuation pathway. The heating of the evacuation pathway can work to prevent significant clogging of the evacuation pathway such that pressure does not build up within the cavity. Nevertheless, as temperatures within the cavity drop further during the cryoablation process, the pressure within the cavity sees a slight increase. This slight increase, however, may be within an acceptable range that does not cause complications during the cryoablation process. Thus, heating the evacuation pathway can substantially prevent clogging of the evacuation pathway, thereby avoiding complications associated with a cryoablation process.

While heating the evacuation pathway as described herein can reduce the likelihood or extent of clogging of an evacuation pathway, a heated lumen construction can increase the complexity of the ablation catheter design and require a larger diameter catheter. In some embodiments, additional or alternative methods to reduce the likelihood of clogging an evacuation pathway can be used. For example, in some embodiments, fluids and moisture can be removed from a cavity or the freezing point of such fluids can be reduced such that, during cryoablation, such fluids do not freeze and lead to particle formation and clogging. Such may involve the introduction of a dehumidification medium or anti-freeze into the cavity before cryoablation or during cryoablation. In some embodiments, a dehumidification or anti-freeze agent may be used in combination with a heated evacuation pathway. Alternatively, a dehumidification or anti-freezing agent may be used in lieu of having a heating evacuation pathway, which can reduce the complexity of the design of an ablation catheter.

Any suitable dehumidification or anti-freezing agent may be used. In some embodiments, the agent may include a solid dehumidification medium or desiccant such as, for example, silica gel, activated charcoal, activated alumina, montmorillonite clay, calcium sulfate, calcium chloride, a molecular sieve (e.g., a zeolite), any other suitable solid desiccant or any suitable combination thereof. In some embodiments, the solid dehumidification medium may be introduced into the internal volume of the internal organ via a catheter so as to dehumidify the internal volume, and later aspirated out of the internal volume (e.g., before or after cryoablation).

In some embodiments, the solid dehumidification medium may be disposed on or embedded into a tip of a cryoablation catheter (e.g., any of the cryoablation catheters described herein). Such an implementation may beneficially allow the solid dehumidification medium to be introduced into a cavity along with the tip of the cryoablation catheter so that a separate introduction step for introducing the solid dehumidification is obviated. Furthermore, the solid dehumidification medium can continue to dehumidify the cavity during the cryoablation process to reduce formation of fluid particles and clogging of the evacuation lumen.

In some embodiments, the dehumidification or anti-freezing agent can include a liquid and/or gas-based medium. It can be desirable to use a liquid dehumidification agent over solid dehumidification agents, as such can mix with and dilute freezable fluids that are within a body lumen, thereby reducing their freezing point and the risk of freezing during a cryoablation procedure. Moreover, liquid dehumidification agents add less thermal mass than solid dehumidification agents, thereby detracting less from the heat transfer required for cryoablation. With liquid dehumidification or anti-freezing mediums, a small quantity of the liquid or gas-based medium, for example, in a range of about 0.1 ml to about 5 ml, inclusive (e.g., about 0.1, 0.2, 0.3, 0.4, 0.5, 1.0, 1.5, 2.0, 2.5, 3.0, 3.5, 4.0, 4.5, 5.0, or 10.0 ml inclusive of all ranges and values therebetween), may be introduced into a cavity prior to cryoablation and/or during cryoablation. The liquid or gas-based medium may be aspirated from the cavity before the cryoablation medium is introduced into the cavity, or left into the cavity during cryoablation. Leaving at least some volume of the liquid or gas-based medium in the internal volume during cryoablation can beneficially continue to remove or prevent freezing of moisture or liquids that may be naturally released by surrounding tissue during cryoablation, e.g., to reduce particle formation and clogging of an evacuation lumen. A liquid or gas-based medium can also be introduced into the internal volume and aspirated therefrom easily.

Examples of liquid or gas-based dehumidification or anti-freezing medium that can be used include ethanol, glycols (e.g., ethylene glycol, diethylene glycol, triethylene glycol, tetraethylene glycol, or propylene glycol), halide salt solutions (e.g., lithium chloride solution, calcium chloride solution, lithium bromide solution, magnesium chloride solution, etc.), alcohols (e.g., ethanol, methanol, isopropanol, or any combination thereof) any other suitable gaseous, liquid dehumidification medium or a combination thereof. In particular, ethanol may be a desirable agent to use. Ethanol provides the benefit of being biocompatible and being a fast-acting desiccant that can rapidly dehumidify the internal volume of an internal organ. Moreover, ethanol has a freezing point of less than −100 degrees Celsius, which when combined with other fluids within the internal volume, can lower the freezing point of such fluids and allow for them to remain liquids during a cryoablation process (e.g., in a range of about −20 to about −50 degrees Celsius). In other words, ethanol can serve as both a dehumidification agent and an anti-freezing agent. In some embodiments, for a body cavity such as the gallbladder, a volume of the ethanol introduced into the cavity may be in a range of about 0.5 mL to about 10 mL, inclusive (e.g., about 0.5, about 5.0, or about 10 mL, inclusive of all ranges and values therebetween).

When performing a cryoablation procedure within a body lumen, such as, for example, the gallbladder, high relative humidity and/or fluids within the body lumen can lead to freezing, which in turn can result in the formation of ice particles or other solids. While it may be possible to aspirate some fluids out of the body lumen prior to a cryoablation procedure to reduce to formation of ice particles or solids, such aspiration typically cannot remove all of the fluids and can have a tendency to cause bleeding and/or undesirable tissue removal. Therefore, the use of a dehumidification medium can help prevent freezing by dehumidifying the internal volume of the body lumen, e.g., by pulling moisture out of the air, prior to the cryoablation procedure and/or during the cryoablation procedure. In some embodiments, the dehumidification medium can also be introduced or delivered into the body lumen such that it is distributed within the lumen and/or mixes with fluids within the lumen. If the dehumidification medium has a lower freezing point than the fluids within the lumen, the dehumidification medium can also suppress the freezing point of the fluids, further reducing the formation of ice particles or solids.

In systems and methods described herein, a dehumidification medium can be introduced into the body lumen before and/or during a cryoablation procedure. By introducing the dehumidification medium, the dehumidification medium can aid in dehumidifying liquid vapors, diluting freezable liquids (thereby decreasing their freezing point), and cleaning the body lumen. But while using a dehumidification medium can provide certain advantages, introducing too high an amount of dehumidification medium may also impact the cooling power that is delivered to the tissue, e.g., because the cryoablation medium would need to cool any dehumidification medium that is introduced into the body lumen. Therefore, in implementations described herein, it can be desirable to have a dehumidification agent that can dissolve or mix with fluids within the body lumen (e.g., to suppress their freezing point) and pull moisture from the air within the body lumen, while enable heat transfer (e.g., for enabling tissue cooling and ablation). In some embodiments, the dehumidification agent can depress the freezing point of the fluids within the body lumen by about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., or about 115° C., inclusive of all values and ranges therebetween.

In addition to limiting heat transfer, too much dehumidification agent in the body lumen can create a mechanical obstruction. Too much dehumidification agent can also pose a risk to evacuation of the dehumidification agent/cryoablation medium mixture. Too much fluid in the body lumen can choke the evacuation and cause transient pressure spikes due to the higher viscosity of the liquid mixture relative to gases within the body lumen. In addition to causing transient pressure spikes, an excessive amount of a dehumidification agent can be pushed out of the exhaust pathway with the pressure gradient.

In some embodiments, the amount of dehumidification agent that is delivered can be within predetermined volumes that enable a cryoablation system (e.g., structurally/functionally similar to any of the cryoablation systems or devices described herein) to achieve interluminal temperatures down to about −60 degrees Celsius. In some embodiments, the amount of dehumidification agent delivered can be based on a size or volume of a body lumen, e.g., to avoid mechanical obstruction using a cryoablation system (e.g., structurally/functionally similar to any of the cryoablation systems or devices described herein). For example, the unexpanded or undistended volume of a body lumen such as a gallbladder can be between about 20 mL and about 100 mL, and the volume of dehumidification agent can be between about 1% and about 50% of the volume of the body lumen, or between about 0.2 mL and about 50 mL, inclusive of all ranges and values therebetween, including, for example, between about 1 mL and about 10 mL.

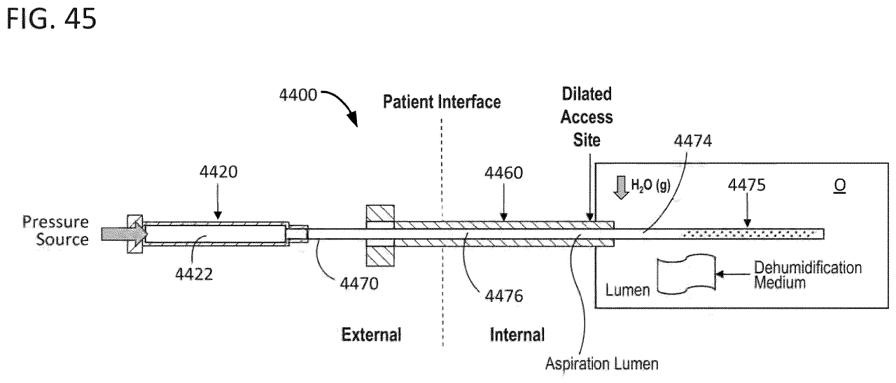

In some embodiments, a dehumidification or anti-freezing medium (e.g., ethanol) may be introduced into the internal volume of the internal organ and/or fluids may be aspirated out of the internal volume using a dedicated system. For example, FIG. 45 is an illustration of a system 4400 for dehumidifying an internal volume of an organ prior to or during cryoablation of the organ, according to an embodiment. The system 4400 can include a shaft 4470 that can be delivered into the internal volume via an introducer or outer shaft 4460. The outer shaft 4460 and/or shaft 4470 can be formed of a polymer, metal, ceramic, or composite or any combination of. A tip of the outer shaft 4460 is configured to be disposed within the internal volume of an internal organ O (e.g., the gallbladder) of a patient either laparoscopically (e.g., by forming an incision in the epidermal layer and adjacent tissue of the patient) or through an intraluminal pathway.

The shaft 4470 is selectively and removably disposed through the lumen defined by the outer shaft 4460 such that a distal a tip 4474 of the shaft 4470 can extend through the lumen of the outer shaft 4460 and be disposed in the internal volume of the organ O. The shaft 4470 defines a channel though which a dehumidification or anti-freezing medium (e.g., ethanol or other suitable mediums as described above) can be communicated into the internal volume of the organ O. In some embodiments, the tip 4474 of the shaft 4470 can define a plurality of openings 4475 (e.g., nozzles or fenestrations) through which the dehumidification or anti-freezing medium can be communicated into the internal volume. In some embodiments, the inner shaft 4470 can be configured to deliver into the internal volume about 0.5 mL to about 10 ml of a liquid dehumidification or anti-freezing medium, inclusive of all sub-ranges and values therebetween.

In some embodiments, the shaft 4470 can serve as an aspiration catheter, e.g., for aspirating fluids and/or other substances out of the internal volume of the organ O. For example, prior to performing a cryoablation procedure in the gallbladder, it can be desirable to aspirate a significant portion of the fluids out of the gallbladder such that those fluids do not remain and cannot freeze during the cryoablation procedure. Therefore, in some embodiments, the shaft 4470 can be used to aspirate fluids and/or other substances out of the internal volume, in addition to or in lieu of delivering a dehumidifying or anti-freezing agent.

A proximal end of the shaft 4470 can be couped to a dehumidification or anti-freezing medium delivery system 4420 (hereinafter “system 4420”). The system 4420 can include a reservoir 4422 configured to store a volume of the dehumidification or anti-freezing medium and fluidically coupled to the shaft 4470. The system 4420 can include a pressure source (e.g., low pressure injection source) that can be fluidically coupled to the reservoir 4422 and configured to exert a positive pressure on the dehumidification or anti-freezing medium contained within the reservoir 4422 to communicate the dehumidification or anti-freezing medium to the internal volume via the shaft 4470, and/or exert a negative pressure on the reservoir 4422 or the inner shaft 4470 to draw out or aspirate fluids from the internal volume. In some embodiments, the system 4420 may be implemented as a syringe. In some embodiments, the pressure source may include a peristaltic pump, a positive displacement pump, a vacuum pump, any other suitable pressure source or a combination thereof. In some embodiments, the dehumidification or anti-freezing medium delivery system 4420 or at least a portion thereof can be housed in a handle assembly (e.g., handle assembly 801).

In operation, the dehumidification or anti-freezing medium can be primed through the shaft 4470, prior to insertion of a cryoablation catheter (e.g., through the introducer or outer shaft 4460). The dehumidification or anti-freezing medium (e.g., ethanol) can have a high partial pressure compared to other fluids within the internal volume such that when the medium is introduced into the cavity, it can reduce relative humidity within the cavity by displacing water vapor and acting as a solvent. In some embodiments, a freezing point of the dehumidification or anti-freezing medium (e.g., ethanol) can be below cryoablation temperatures such that the dehumidification or anti-freezing medium remains in liquid form during ablation and acts as an anti-freeze. The dehumidification or anti-freezing medium can be delivered to internal volume of the organ O and left in place, followed by placement of an ablation catheter, such as any of the ablation catheters or assemblies described herein (e.g., inner shaft 270, ablation catheter assembly 3570). In some embodiments, a seal can be disposed between the introducer or outer shaft 4460 and the shaft 4470, while in other embodiments, there does not need to be a seal between the outer shaft 4460 and the shaft 4470.

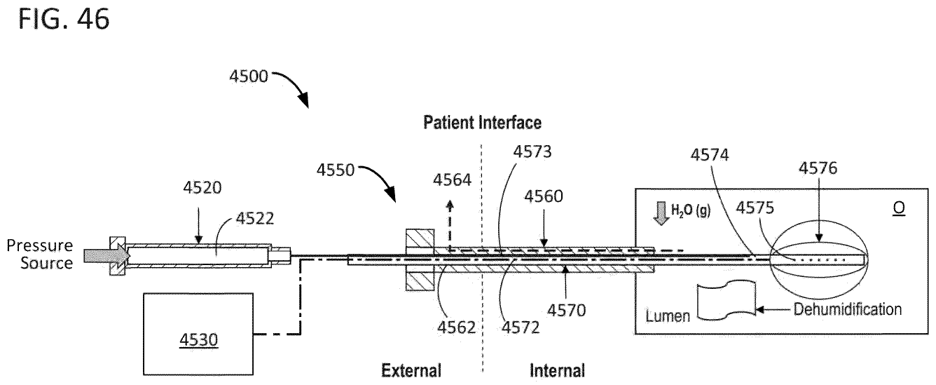

In some embodiments, a dehumidification system may be integrated with or combined with a cryoablation system. In particular, a dedicated lumen priming system may be provided. For example, FIG. 46 is an illustration of an ablation system 4500 that includes a catheter assembly 4550 for communicating a dehumidification medium (e.g., ethanol) and a cryogenic ablation medium (e.g., liquid nitrogen) into an internal volume of an organ O or other body cavity, according to an embodiment. The ablation system 4500 can have a dedicated priming lumen design. The ablation system 4500 can include components that are functionally and/or structurally similar to those of other ablation systems described herein.

The catheter assembly 4550 includes a shaft 4570 that can be inserted through a lumen 4562 of an introducer 4560. In some embodiments, the shaft 4570 can be an inner shaft, e.g., similar to other inner shafts described herein, and the introducer 4560 can be an outer shaft, e.g., similar to other outer shafts described herein. The shaft 4570 can have an outer diameter that is substantially less than an inner diameter of the introducer 4560 such that an evacuation pathway 4564 is defined between an inner radial surface of the introducer 4560 and an outer radial surface of the shaft 4570. The evacuation pathway 4564 can allow fluids, cryogenic ablation medium and/or other particles to be evacuated from the internal volume of the organ O. In some embodiments, the introducer 4560 may define separate lumens for the shaft 4570 and the evacuation pathway 4564, e.g., similar to that described with reference to FIG. 42 above. In some embodiments, the introducer and/or the shaft 4570 may include lumens for pressure sensing and/or temperature sensing as previously described herein.

The introducer 4560 and/or shaft 4570 can be formed of a polymer, metal, ceramic, composite or any combination of. A tip of the introducer 4560 is configured to be disposed within the internal volume of an internal organ O (e.g., the gallbladder) of a patient either laparoscopically or through an intraluminal pathway.

The shaft 4570 is selectively and removably disposed through the lumen 4562 defined by the introducer 4560 such that a distal tip 4574 of the shaft 4570 can extend through the lumen 4562 of the introducer 4560 and be disposed in the internal volume of the organ O. The shaft 4470 defines a first channel 4572 through which a cryogenic ablation medium is communicated to the internal volume of the organ O for cryoablating the organ O. A proximate end of the first channel 4572 is fluidically coupled to a cryogenic medium delivery system 4530 that may include a reservoir storing the cryogenic ablation medium (e.g., liquid nitrogen). In some embodiments, the cryogenic medium delivery system 4530 or at least a portion thereof can be housed in a handle assembly (e.g., handle assembly 801). In some embodiments, the cryogenic ablation medium may be stored under high pressure (e.g., in a compressed gas cylinder). In some embodiments, the shaft 4570 includes a plurality of openings 4575 (e.g., nozzles or fenestrations) defined on the tip 4574, and the cryogenic ablation medium can be delivered into the internal volume or cavity through the plurality of openings 4575. In some embodiments, an expandable structure 4576 can be disposed on or near the tip 4574. The expandable structure 4576 can be the same or substantially similar to other expandable structures described herein, including, for example, expandable structures 876, 3176, 3576, etc., or any other expandable structure described herein. Thus, certain aspects of the expandable structure 4576 are not described in greater detail herein.

In some embodiments, the shaft 4570 also defines a second channel 4573 though which a dehumidification or anti-freezing medium (e.g., ethanol) can be communicated into the internal volume of the organ O. In some embodiments, the inner shaft 4570 can be configured to communicate about 0.5 mL to about 10 mL, inclusive of sub-ranges and values therebetween, of the liquid dehumidification or anti-freezing medium into the internal volume. The dehumidification or anti-freezing medium can be delivered as fluid, gas, or in an aerosolized form.

The second channel 4573 may be fluidically couped to a dehumidification or anti-freezing medium delivery system 4520 that can include a dehumidification or anti-freezing medium reservoir 4522 and a pressure source. The dehumidification or anti-freezing medium delivery system 4520 may be substantially similar to the system 4420 and, therefore, not described in further detail herein.

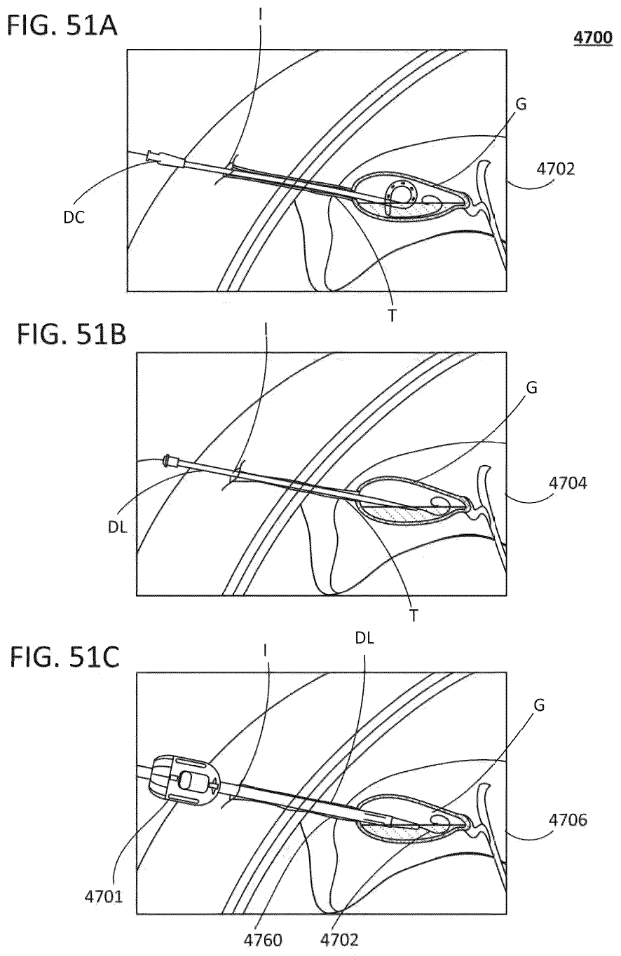

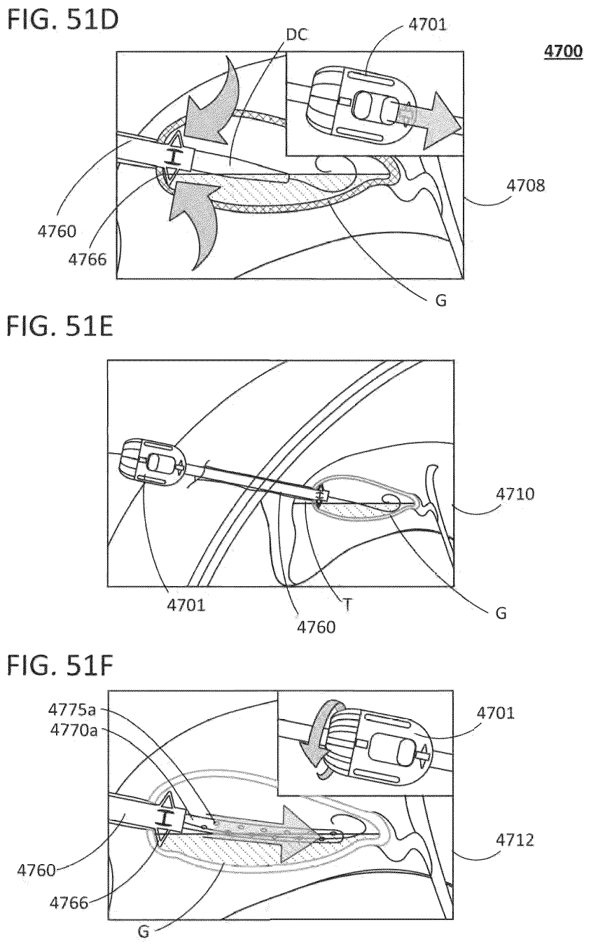

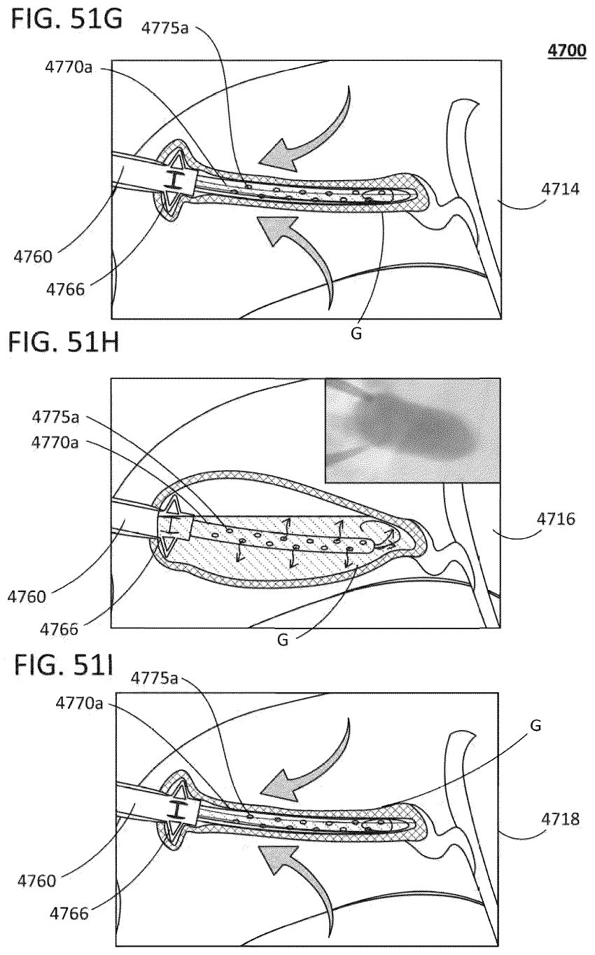

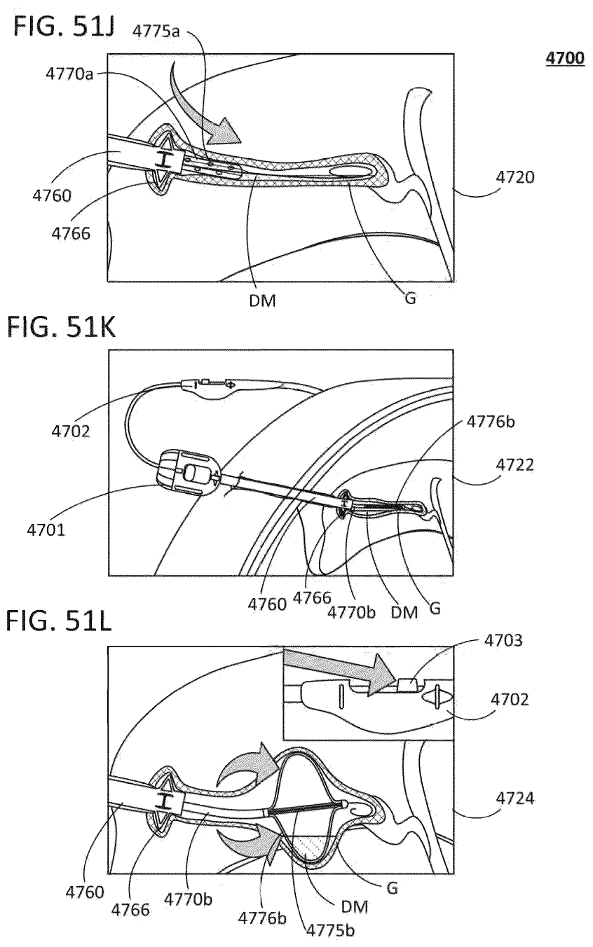

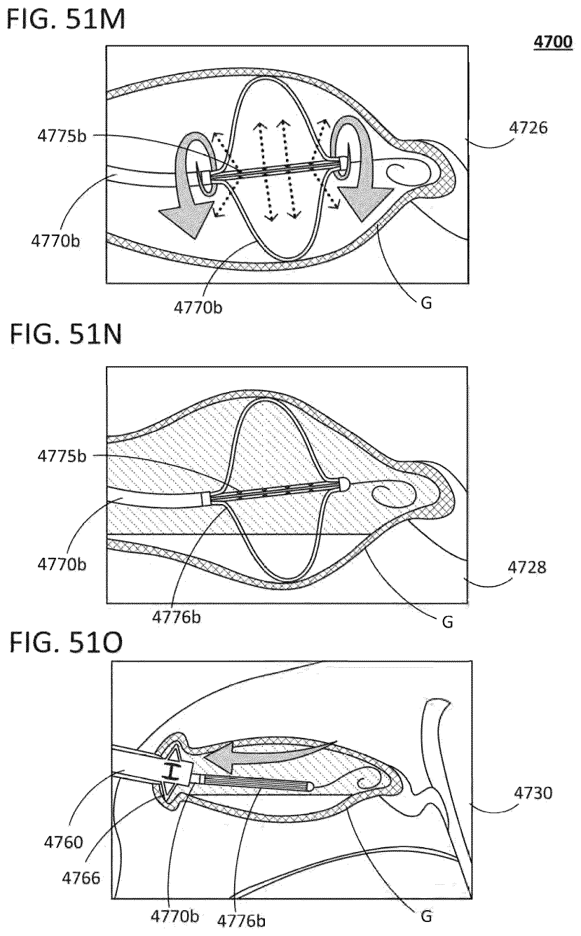

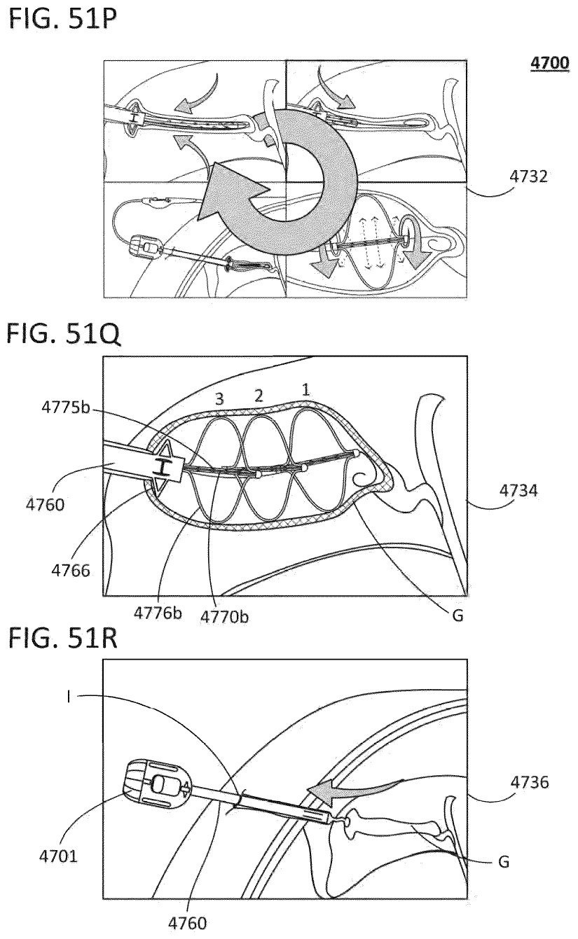

In operation, the second channel 4573 provides a dedicated pathway to inject the dehumidification or anti-freezing medium from the reservoir 4522 into the target ablation lumen prior to ablation. In some embodiments, low pressure positive driving pressure of the dehumidification medium delivery system 4520 can be manually or electronically generated (e.g., via a syringe, injection pump, compressed gas, etc.). In some embodiments, the driving pressure of the dehumidification medium may be less than about 100 psi. In some embodiments, after delivering the dehumidification or anti-freezing agent, fluids and/or other substances within the internal volume or body cavity can be aspirated, e.g., using an aspiration device as described later with reference to FIGS. 51A-R. The cryogenic ablation medium can then be delivered separately through the first channel 4752 for cryoablating the organ O. In some embodiments, the system 4520 or at least a portion thereof can be housed in a handle assembly (e.g., handle assembly 801).

While the ablation system 4500 is described as having separate lumens for delivery a cryogenic ablation medium and a dehumidification or anti-freezing agent, it can be appreciated that in some embodiments, a single lumen can be used to deliver both the cryogenic ablation medium and a dehumidification or anti-freezing agent. In such embodiments, one or more valves can be configured to control selective delivery of the cryogenic ablation medium and the dehumidification or anti-freezing agent.

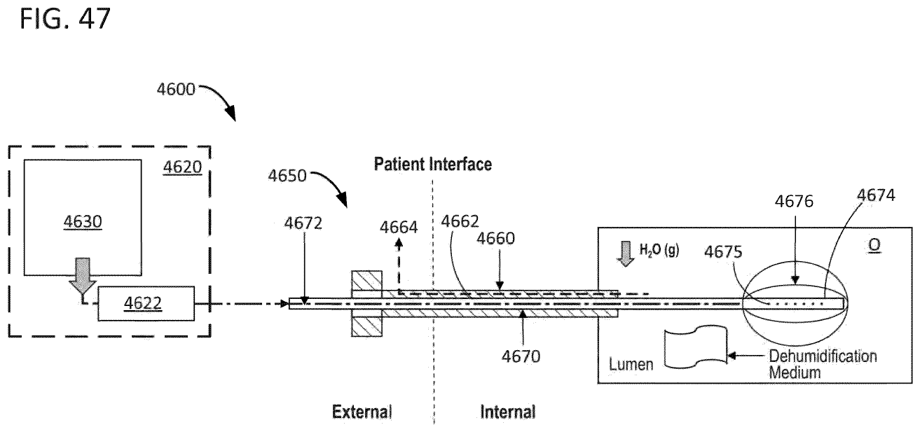

In some embodiments, an integrated cryogen priming system may be provided. The integrated cryogen priming system can be configured to deliver a mixture of a cryogenic ablation medium and a dehumidification or anti-freezing medium to an internal volume of an organ or other body cavity. For example, FIG. 47 is an illustration of an ablation system 4600 that includes a catheter assembly 4650 for communicating a mixture of a dehumidification or anti-freezing medium and a cryogenic ablation medium into an internal volume of an organ O, according to an embodiment.

The catheter assembly 4650 includes a shaft 4670 that can be inserted through a lumen 4662 of an introducer 4660. In some embodiments, the shaft 4670 can be an inner shaft, e.g., similar to other inner shafts described herein, and the introducer 4660 can be an outer shaft, e.g., similar to other outer shafts described herein. The shaft 4670 can have an outer diameter that is substantially less than an inner diameter of the introducer 4660 such that an evacuation pathway 4664 is defined between an inner radial surface of the introducer 4660 and an outer radial surface of the shaft 4670 through which fluids can be evacuated from the internal volume of the organ O or other body cavity. In some embodiments, the introducer 4660 may define separate lumens for the shaft 4670 and the evacuation pathway 4664. In some embodiments, the introducer 4660 and/or the shaft 4670 may include lumens for pressure sensing and/or temperature sensing as previously described herein.

The introducer 4660 and/or shaft 4670 can be formed of a polymer, metal, ceramic, or composite or any combination of. A tip of the introducer 4660 is configured to be disposed within the internal volume of the organ O. The shaft 4670 is selectively and removably disposed through the lumen 4662 defined by the introducer 4660 such that a distal tip 4674 of the inner shaft 4670 extends through the lumen 4662 of the introducer 4660 and is disposed in the internal volume of the organ O. The shaft 4670 defines a channel 4672 through which a mixture of cryogenic ablation medium (e.g., liquid nitrogen) and a dehumidification or anti-freezing medium (e.g., ethanol) is communicated to the internal volume of the organ O for cryoablating the organ O while preventing ice-formation or freezing. In some embodiments, a plurality of openings 4675 (e.g., nozzles or fenestrations) can be defined on the tip 4674 through which the cryogenic ablation medium and dehumidification or anti-freezing agent can be communicated into the internal volume. In some embodiments, an expandable structure 4676 can be disposed on or near the tip 4674. The expandable structure 4676 can be substantially similar to other expandable structures described herein, including, for example, the expandable structure 4676, and therefore not described in further detail herein.

A proximate end of the channel 4672 is fluidically coupled to a delivery system 4620. The delivery system 4620 can include an ethanol reserve 4622 and a cryogen source 4630. In some embodiments, the delivery system 4620 can be a high-pressure injection system. In some embodiments, the delivery system 4620 can be configured to deliver a non-homogenous or homogeneous mixture of cryogen and the dehumidification or anti-freezing agent. For example, the delivery system 4620 can include a high-pressure source of cryogen, which can be used to propel dehumidification or anti-freezing agent located within the ethanol reserve 4622 into the body cavity followed by delivery of the cryogen. In such embodiments, the ethanol reserve can be coupled to a cartridge containing the cryogen shortly before use. In some embodiments, the system 4620 or at least a portion thereof can be housed in a handle assembly (e.g., handle assembly 801). Alternatively, the cryogen and the dehumidification or anti-freezing agent can be pre-mixed, and can be delivered together into the body cavity. In some embodiments, the high-pressure source of cryogen (e.g., a liquid or compressed gas cylinder) can be configured to pressurize the cryogenic ablation medium at a pressure of about 500 psi to about 1,000 psi, inclusive (e.g., about 500, 550, 600, 650, 700, 750, 800, 850, 900, 950, or about 1,000 psi inclusive of all ranges and values therebetween).

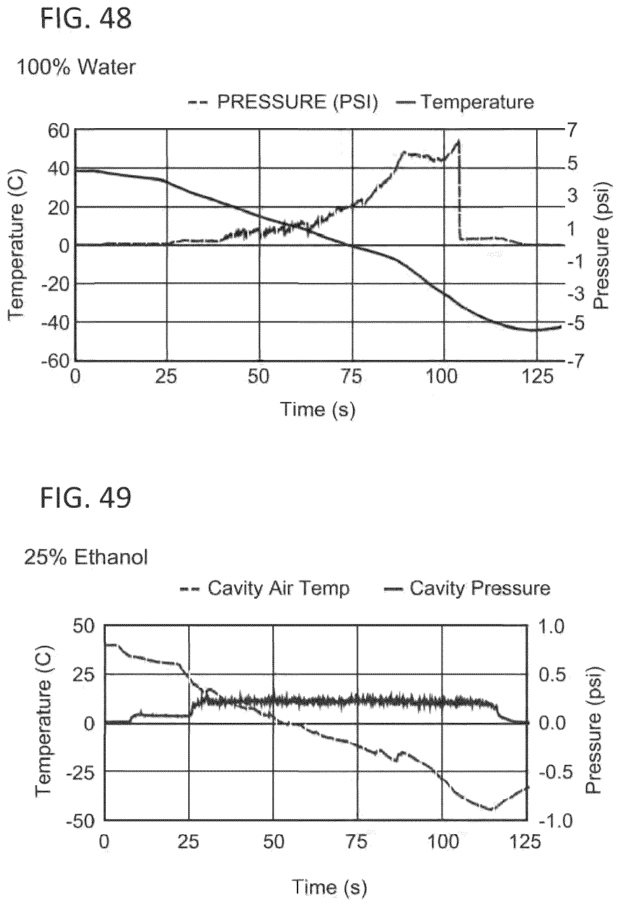

FIGS. 48-50 depict pressure and temperature of a cavity of a vapor load model used to simulate a body cavity or organ undergoing a cryoablation process, where different amounts of ethanol are used. FIG. 48 depict pressures and temperatures over time, when no ethanol has been used (i.e., when the humidity in an internal volume of the organ is about 100%). As observed in FIG. 48, decrease in temperature below zero degrees causes an associated increase in pressure in the body cavity due to freezing of moisture present in the body cavity that forms ice particles in the evacuation pathway. The ice particles progressively block the evacuation pathway resulting in restriction of flow of fluids within the body cavity through the evacuation pathway and corresponding increase in pressure in the body cavity as the temperature drops below 0 degrees Celsius. As such, without the use of a dehumidification or anti-freezing agent such as ethanol, the body cavity can reach very high pressures, which may cause complications and injury, as described above.

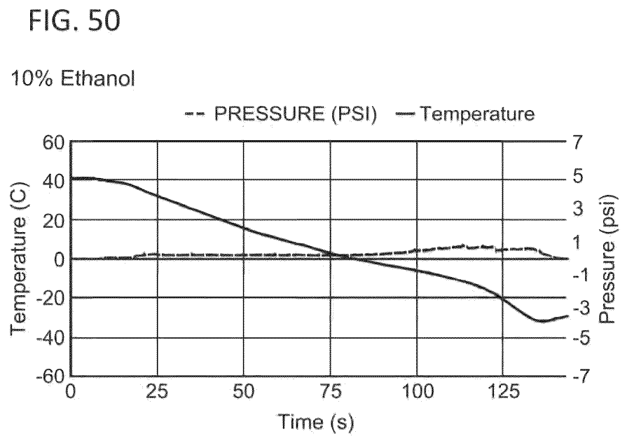

FIG. 49 depicts pressures and temperatures over time in a body cavity, when the body cavity has been dehumidified with ethanol at a 4:1 ratio. In particular, the cavity of the vapor load model was filled with ethanol equal to about 25% of the amount of water in the cavity. And FIG. 50 depicts pressures and temperatures over time in a body cavity, when the body cavity has been dehumidified with ethanol at a 10:1 ratio. In particular, the cavity of the vapor load model was filled with ethanol equal to about 10% of the amount of water in the cavity. After filling the cavity with the respective amounts of ethanol, cryogen was then introduced into the cavity to perform the cryoablation.

As shown in FIGS. 49 and 50, priming the internal volume of the organ (e.g., gallbladder) with ethanol at dilutions or ratios of 25% (FIG. 49) or even 10% (FIG. 50) can reduce or prevent the formation of ice particles, which can lead to clogging to the evacuation pathway. As such, the use of ethanol or other dehumidification or anti-freezing agents can maintain sufficient gas flow to keep pressures below a threshold evacuation pressure (e.g., less than 1 psi) in the evacuation pathway.

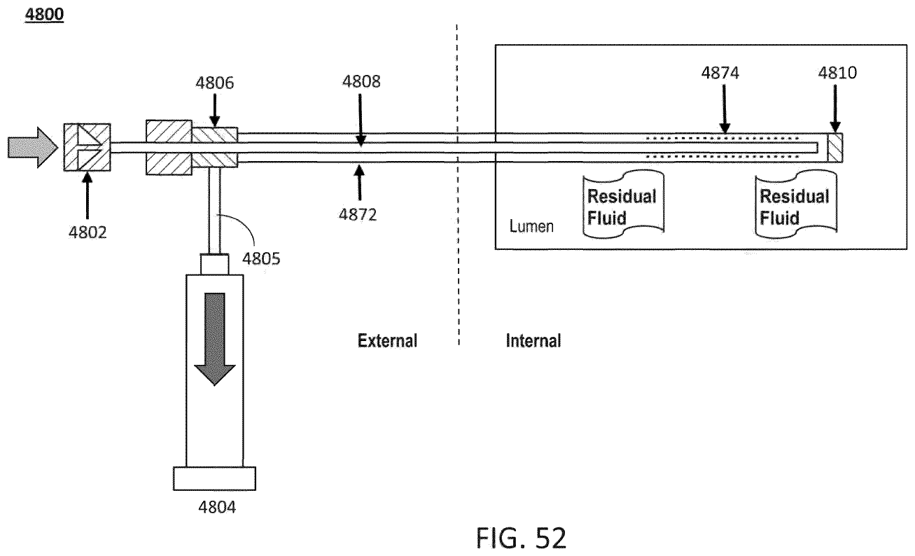

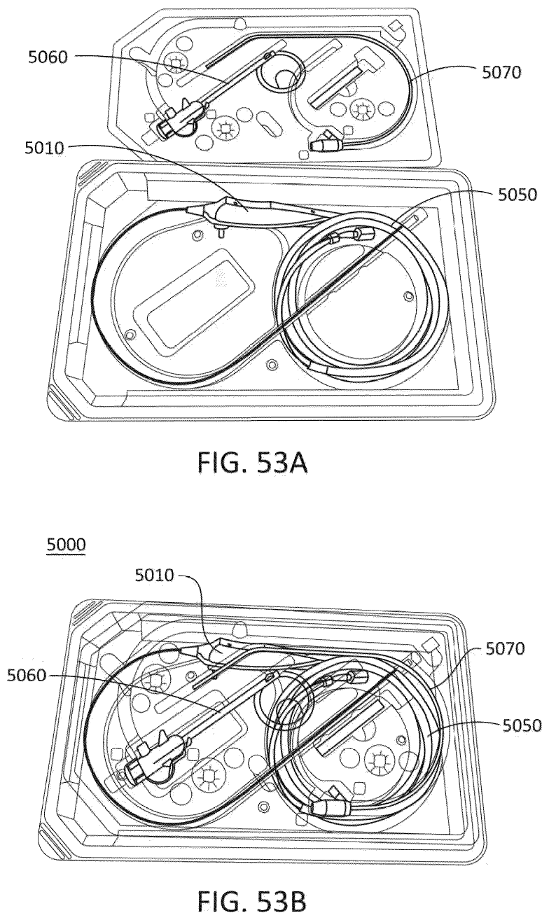

FIGS. 52-53B depict examples of aspiration catheters that are used with ablation systems described herein, according to embodiments. Aspiration catheters that apply high suction and/or have rough finish on their surfaces or holes may lead to trauma in some cases, which can cause excessive bleeding throughout the during of a procedure. In some cases, a clinician using an aspiration catheter may also pull or adjust an aspiration catheter while applying aspiration, which can potentially further cause trauma to a gallbladder lumen or other body cavity lumen. In some embodiments, aspiration catheters may also create a seal with a distal tapered tip of an introducer or outer shaft (e.g., similar to the introducers and outer shafts of the ablation systems described herein). For example, an aspiration catheter that is 14 Fr in size may have an outer diameter that is substantially similar to an inner diameter of an introducer or outer sheath, and therefore form a seal. While the aspiration applied by such aspiration catheters may be effective, it can be desirable to include features that limit the amount of suction being applied, e.g., for safety and to reduce bleeding.

FIG. 52 depicts an aspiration catheter 4800, according to an embodiment. The aspiration catheter 4800 can include an aspiration lumen 4872, which can be coupled to a vacuum or suction source 4804. In some embodiments, the vacuum source 4804 can be implemented as a syringe. The vacuum source 4804 can be coupled to the aspiration lumen via a connector or tubing 4805. The aspiration lumen 4872 can terminate in one or more openings or fenestrations 4874, which can be disposed in a body cavity. As such, when vacuum is applied by the vacuum source 4804, suction can be applied to the cavity to aspirate or suction fluids and other substances out of the body cavity. To prevent over-pressure, the aspiration catheter 4800 can include a relief lumen 4808 that can limit the total amount of vacuum applied to an aspiration lumen, e.g., by opening or breaking the seal of a valve 4802 (e.g., a check valve) at a preset pressure. The relief lumen 4808 can be in fluid communication with the aspiration lumen 4872. In some embodiments, the relief lumen 4804 can be concentrically disposed within the aspiration lumen. The aspiration catheter 4800 can include a distal end 4810 that is sealed and can also include a seal 4806 (e.g., a Tuohy seal) that is configured to seal around the relief lumen 4808. In use, when aspiration or suction is being applied, if the pressure generated within the relief lumen 4808 is greater than a preset value, then the seal or valve 4802 coupled to the relief lumen 4808 can open, to allow for pressure release. In some embodiments, a fluid (e.g., the dehumidification agent, a contrast agent, air, saline, or other gases or liquids) can be delivered to the aspiration lumen 4872 via the tubing 4805. In other words, instead of the vacuum source 4804 being coupled to the tubing 4805, a source of the fluid (e.g., a syringe containing the fluid) can be coupled to the tubing 4805 and used to inject or deliver the fluid through the aspiration lumen 4872 into the body cavity. With such operation, the aspiration lumen 4872 (and coupled components) can function in both directions (i.e., for fluid delivery and aspiration). In some embodiments, one or more additional lumens can be added to the aspiration catheter 4800 for delivery of fluids.

FIGS. 53A and 53B depict a prototype of an aspiration catheter 4900, according to an embodiment. The aspiration catheter 4900 can be structurally and/or functionally similar to the aspiration catheter 4800. For example, the aspiration catheter 4900 can include an aspiration lumen 4872, a relief lumen 4908, a plurality of openings 4974, a line 4905 that is configured to couple to a vacuum source, a seal 4906 that seals around the relief lumen 4908, and a check valve 4902 in communication with the relief lumen. As shown the relief lumen 4908 and the aspiration lumen 4972 are concentrically disposed. The distal end of the relief lumen 4908 and the aspiration lumen 4972 (as shown in greater detail in FIG. 53B) can be disposed within a body cavity, e.g., for applying suction.

FIGS. 51A-51R are schematic illustrations of various operations of a method 4700 for dehumidifying and cryoablating a gallbladder, according to an embodiment. While the method 4700 is shown as including various operations performed in a particular order, this is for illustrative purposes only. Thus, one or more of the operations shown with respect to the method 4700 can be excluded or performed in a different order than shown in FIGS. 51A-51R, or the method 4700 can include other operations not shown in FIGS. 51A-51R. Moreover, while the operations of the method are shown as being performed by specific catheters or catheter assemblies, this is for example only and any suitable catheters (e.g., any of the aspiration catheters, dehumidification medium delivery and/or cryogen delivery catheters, or other catheter assemblies described herein) can be used to perform the operations of the method 4700. All such variations are contemplated and should be considered to be within the scope of the present disclosure.

The method 4700 can include inserting an aspiration catheter or a drainage catheter (e.g., aspiration catheter 4800, 4900) into a gallbladder G, at 4702 corresponding to FIG. 51A. For example, an incision I may be formed in an epidermal layer and adjacent tissue of a subject and a drainage catheter DC inserted therethrough via into an internal volume of a gall bladder G, for example, through a mature tract T formed in the patient that leads to the gall bladder G. The drainage catheter DC may be used to aspirate the gallbladder G, for example, by draining out bile that may be present in the gallbladder.

At 4704 corresponding to FIG. 51B, a dilator DL (e.g., the dilator 3068) can be inserted through the tract T, for example, over the drainage catheter DC, or subsequent to removing the drainage catheter DC from the tract T. The dilator DL is used to dilate the tract T to expand the tract to a predetermined size (e.g., in range of about 16 Fr to about 20 Fr).

At 4706 corresponding to FIG. 51C, an introducer or outer shaft 4760 is advanced along a guidewire 4702 with the dilator positioned within a lumen of the outer shaft 4760. The introducer 4760 may be substantially similar to the outer shaft or introducers 4460, 4560, 4760, or any other outer shafts or introducers described herein. A first handle 4701 is coupled to a proximate end of the introducer 4760 and is used to control the operations of the introducer 4760 (e.g., via rotation or lateral movement of the first handle 4701, via any other suitable controls provided in the handle 4701 or a combination thereof). In some embodiments, a series of progressively larger dilators can be advanced along the guidewire 4702 to dilate the tract T into the body lumen before advancing the introducer 4760 into the gallbladder. In some embodiments, placement of the outer shaft 4760 can be similar to a percutaneous drainage tube placement technique.

The introducer 4760 includes a first expanding structure 4766 disposed proximate to a tip of the introducer 4760 that is disposed within the gallbladder G. At 4708 corresponding to FIG. 51D, the first expanding structure 4766 is deployed to secure or retain the tip of the introducer 4760 within the internal volume of the gallbladder G. The first expandable structure 4766 may be substantially similar to the expandable structure 266, 866, 1066, 1266, 1366, 1566, 1766, 1866, 1866′, 3066, 3166, 3266, or any other expandable structure configured to retain the tip of the introducer 4766 within the internal volume of the gall bladder G, as previously described herein.

At 4710 corresponding to FIG. 51E, the dilator DL is removed from the tract T by retracting the dilator DL from the lumen of the introducer 4760. At 4712 corresponding to FIG. 51F, a first shaft 4770a (e.g., an aspiration catheter) is inserted through the lumen of the introducer 4760 until a tip of the first shaft 4770a is disposed in the internal volume of the gallbladder G. The first shaft 4770a defines a plurality of openings 4775a at the tip thereof. In some embodiments, the first shaft 4770a may be threaded into the introducer 4760 and locked in place via the handle 4701 (e.g., via a rotation of the handle 4701 in a predetermined direction).

At 4714 corresponding to FIG. 51G, a negative pressure or suction is applied to the first inner shaft 4770a to aspirate bile from the gallbladder via the plurality of openings 4775a. In some embodiments, at 4716 corresponding to FIG. 51H, an imaging solution (e.g., a saline/contrast agent solution in a 50/50 concentration) is communicated via the first shaft 4770 a into the gallbladder G to fill the gallbladder G with the imaging solution, and the gallbladder can be visualized (e.g., via ultrasound imaging, a computed tomography (CT) scan, a magnetic resonance imaging (MRI) scan, or a positron emission tomography (PET) scan).

At 4718 corresponding to FIG. 51I, the imaging solution is aspirated from the gallbladder G via the first shaft 4770 a. At 4720 corresponding to FIG. 51J, a volume of a dehumidification or anti-freezing medium DM such as ethanol (e.g., about 1 ml to about 10 ml, inclusive of all values and sub-ranges therebetween, of ethanol) is communicated into the internal volume of the gall bladder G via the first shaft 4770a.

At 4722 corresponding to FIG. 51K, the first shaft 4770 a is withdrawn from the introducer 4760, and a second shaft 4770b is inserted through the lumen of the introducer 4760 such that a tip of the second shaft 4770b protrudes from the tip of the shaft 4760 into the internal volume of the gallbladder G. The second shaft 4770b can be structurally and/or functionally similar to any of the inner shafts and/or ablation catheters described herein., The second shaft 4770b defines a plurality of openings 4775b (FIG. 51L) at the distal tip thereof through which a cryogenic ablation medium can be communicated into the gallbladder G. A second expandable structure 4776b (e.g., an expandable cage) 4776b is disposed on the distal tip of the second shaft 4770b in a collapsed configuration. The second expandable structure 4776b can be substantially similar to the expandable structure 4576, 4676, or any other expandable structure described herein with respect to any of the cryoablation catheters described herein. A second handle 4702 is coupled to a distal end of the second shaft 4770b and configured to control operations of the second shaft 4770b.

At 4724 corresponding to 51L, an engagement element 4703 (e.g., a slider) of the second handle 4702 is engaged by a user to cause the second expandable structure 4776b to transition into an expanded configuration such that elongate members or arms of the second expandable structure 4776b contact walls of the gallbladder G.

At 4728 corresponding to FIG. 51M, a cryogenic ablation medium is communicated into the gallbladder G through the second shaft 4770b via the openings 4775b defined at the tip of the second shaft 4770b, while rotating the second expandable structure 4776b. For example, the cryogenic ablation medium may be N₂O that is communicated at a pressure of less than or equal to 30 mm Hg for a time period of less than or equal to 30 seconds through the second shaft 4776 c into the gallbladder G, so as to cryoablate at least a portion of the gallbladder G. During delivery of the cryogenic ablation medium, the cryogenic ablation medium and/or other fluids within the gallbladder G can be evacuated out from within the gallbladder G via an evacuation pathway or lumen.

At 4728 corresponding to FIG. 51N, the gallbladder G is allowed to thaw for a predetermined time period (e.g., at least about 1 minute or at least about 2 minutes). At 4730 corresponding to FIG. 510, the second expandable structure 4776b is transitioned into the collapsed configuration (e.g., by engaging and retracting the engagement element 4703 of the second handle 4702), and the second shaft 470b is withdrawn from the gallbladder G via the introducer 4760.

At 4732 corresponding to FIG. 51P, operations 4718 to 4730 are repeated for a predetermined number of times (e.g., 2 times, 3 times, 4 times, or about 10 times, including all values and sub-ranges therebetween) to subject the gallbladder G to the predetermined number of cryoablation cycles. At 4734 corresponding to FIG. 51Q, the location of the second expandable structure 4776b in the expanded configuration is changed or shifted between each cryoablation cycle such that the arms of the second expandable structure expand or contact different portions of gallbladder G during subsequent cryoablation cycles to facilitate cryoablation of the entire volume of the gallbladder G. For example, as shown in FIG. 51Q, during a first cryoablation cycle, the second expandable structure 4776b is located at a first position 1 that is most distal from a tip of the outer shaft 4760. During a second cryoablation cycle, the second expandable structure 4776b is located at a second position 2 that is closer to the tip of the outer shaft 4760 relative to the first location 1. Then during a third cryoablation cycle, the second expandable structure 4776b is located at a third position 3 that is closest to the tip of the outer shaft 4760 relative to the first position 1 and the second position 2.

At 4736 corresponding to FIG. 51R, after sufficient cryoablations cycles, the gallbladder G is substantially cryoablated and inactivated, and the second shaft 4770b is retracted from the introducer 4760. The introducer 4760 can be subsequently removed from the subject's body and the incision I closed to complete the procedure.

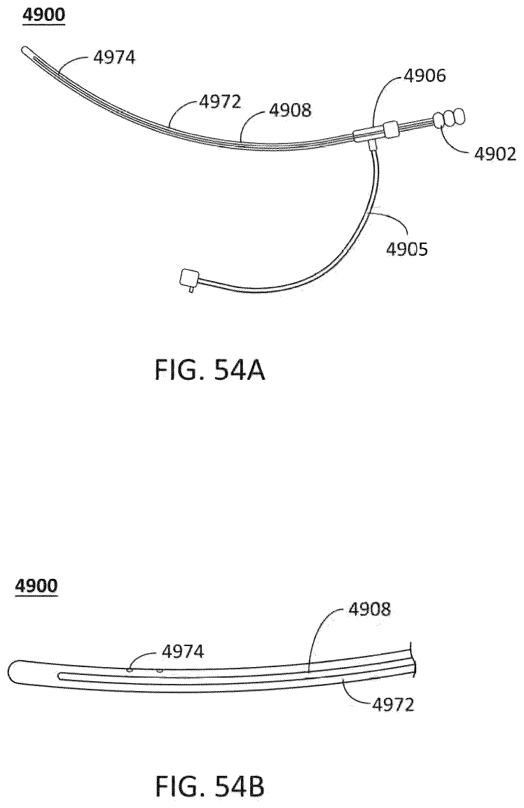

FIGS. 54A-54B are images of a catheter kit for formation of a catheter assembly 5000. FIG. 54A shows the components of the catheter assembly 5000 pre-assembly, while FIG. 54B shows the catheter assembly 5000 post-assembly. As shown, the catheter kit includes a control unit 5010, a cryoablation catheter 5050, an introducer 5060, and an aspiration catheter 5070. In some embodiments, the aspiration catheter 5070 can be the same or substantially similar to the aspiration catheter 4800, as described above with reference to FIG. 52. In some embodiments, the control unit 5010 can be the same or substantially similar to the control unit 310, as described above with reference to FIG. 5. In some embodiments, the cryoablation catheter 5050 can be the same or substantially similar to any of the cryoablation catheters described herein. In some embodiments, the introducer 5060 can be the same or substantially similar to the introducer 4560, as described above with reference to FIG. 46. Thus, certain aspects of the control unit 5010, the cryoablation catheter 5050, the introducer 5060, and the aspiration catheter 5070 are not described in greater detail herein. In some embodiments, the dehumidification agent can be stored in the control unit 5010. In some embodiments, the dehumidification agent can be delivered from the control unit 5010. In some embodiments, the dehumidification agent can be stored in the control unit 5010 and delivered through a lumen to the cryoablation catheter 5050.

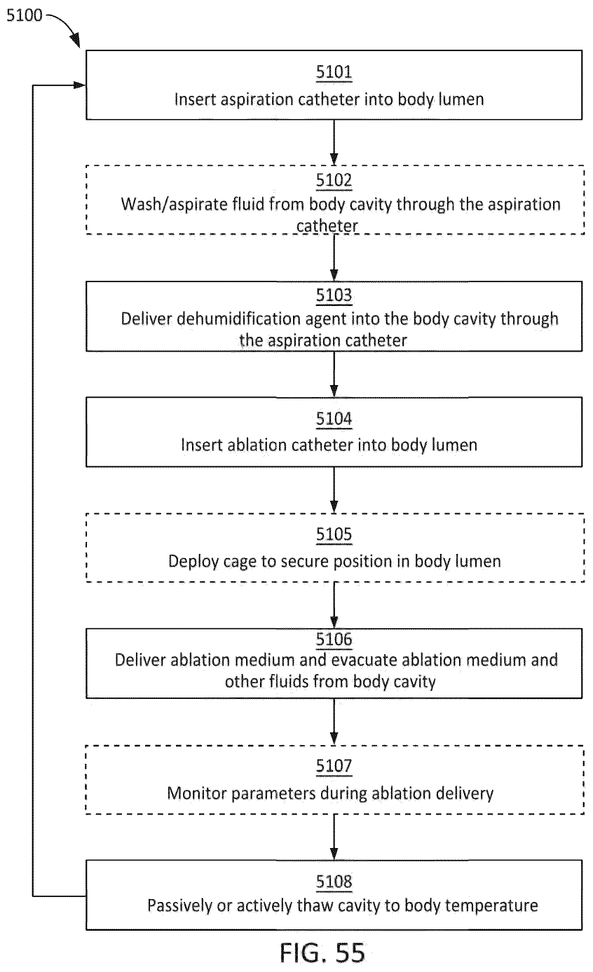

FIG. 55 is a flow diagram of a method 5100 of administration of a cryogenic fluid to a body lumen, according to an embodiment. In some embodiments, the cryogenic fluid can be delivered via a catheter assembly the same or substantially similar to the catheter assembly 5000, as described above with reference to FIGS. 54A-54B. As shown, the method 5100 includes inserting an aspiration catheter into a body lumen at 5101, optionally washing or aspirating fluid from the body cavity at 5102, priming or delivering dehumidification agent into body cavity at 5103, inserting an ablation catheter into a body lumen at 5104, optionally deploying a cage to secure the position of the catheter in the body lumen at 5105, delivering an ablation medium into the body lumen and evacuating fluids from the body lumen at 5106, optionally monitoring one or more parameters during ablation delivery at 5107, and passively or actively thawing the cavity to body temperature at 5108.

In some embodiments, the method 5100 can be repeated at least 1, at least 2, at least 3, at least 4, at least 5, at least 6, at least 7, at least 8, at least 9, or at least 10 times to fully treat (e.g., defunctionalize) and remove all or substantially all debris from the gallbladder lumen.

At 5101, the method 5100 includes inserting an aspiration catheter (e.g., structurally and/or functionally similar to those described herein) into the body lumen. The aspiration catheter can be inserted through a lumen of an introducer or other shaft (e.g., structurally and/or functionally similar to those described herein). In some embodiments, the body lumen can include a gallbladder.

Optionally, at 5102, the method 5100 includes washing or aspirating fluid from the body cavity through the aspiration catheter. For example, the method 5100 may include delivering a wash solution, a contrast agent, or another fluid into the body cavity and/or aspirating fluids within the body cavity. In some embodiments, the fluid can include saline. Saline can be used to lavage the body lumen and clean debris. In some embodiments, the saline can have a salt concentration of about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, about 150 mg/mL, about 200 mg/mL, about 250 mg/mL, about 300 mg/mL, or about 350 mg/mL, inclusive of all values and ranges therebetween. In some embodiments, the prime fluid can include ethanol. Ethanol can be used to dilute freezable fluids and clean debris in the gallbladder. The aspiration of fluids within the body cavity can facilitate removal of fluids that may lead to freezing or other complications during cryoablation, as described herein.

At 5103, the method 5100 includes priming the body cavity with a dehumidification agent or fluid. The fluid can be delivered or injected through the aspiration catheter. In some embodiments, an alcohol (or other low freezing point dehumidification agent) is introduced into the body lumen via the aspiration catheter to dehumidify the cavity and prevent freezing of fluids prior to ablation. In some embodiments, the alcohol can include ethanol. In some embodiments, the alcohol can include methanol. In some embodiments, the alcohol can include isopropyl alcohol. In some embodiments, at least about 500 μL, at least about 1 mL, at least about 1.5 mL, at least about 2 mL, at least about 2.5 mL, at least about 3 mL, at least about 3.5 mL, at least about 4 mL, at least about 4.5 mL, at least about 5 mL, or at least about 10 mL of the alcohol (or other low freezing point dehumidification agent) can be introduced into the body lumen to dehumidify the cavity. In some embodiments, no more than about 10 mL, no more than about 5 mL, no more than about 4.5 mL, no more than about 4 mL, no more than about 3.5 mL, no more than about 3 mL, no more than about 2.5 mL, no more than about 2 mL, no more than about 1.5 mL, or no more than about 1 mL of the alcohol (or other low freezing point dehumidification agent) can be introduced into the body lumen to dehumidify the cavity. Combinations of the above-referenced amounts of the alcohol (or other low freezing point dehumidification agent) are also possible (e.g., at least about 500 μL and no more than about 5 mL or at least about 1 mL and no more than about 4 mL), inclusive of all values and ranges therebetween. In some embodiments, the dehumidification agent or fluid can be injected using a pressure source or pump, such as, for example, a syringe.

At 5104, the method 5100 includes inserting an ablation catheter (e.g., structurally and/or functionally similar to other ablation catheters described herein) into the body lumen. In some embodiments, the ablation catheter and the aspiration catheter can occupy the body lumen simultaneously. In some embodiments, the aspiration catheter can be inserted and removed from the body lumen before inserting the ablation catheter into the gallbladder lumen.

Optionally, at 5105, the method 5100 includes deploying a cage or other expandable structure to approximate the location of the ablation medium delivery the body lumen. For example, as described above, the cage can be configured to approximate the location of a nozzle of the ablation catheter, which can space the nozzle from a wall of a gallbladder, e.g., to enable better distribution of a cryoablation medium that is delivered therethrough. In some embodiments, the cage can expand away from a central axis of the ablation catheter.

At 5106, the method 5100 includes delivering an ablation medium to the body lumen. In some embodiments, the ablation medium can include a cryoablation medium. In some embodiments, the amount of ablation fluid deployed at 5104 can provide total energy of between about 2 kJs and about 10 kJs, inclusive of all values and ranges therebetween. In some embodiments, the ablation fluid can be delivered at a predetermined wattage for a predetermined period of time. For example, the ablation fluid can be delivered at between about 40 and about 200 Watts, including all values and ranges therebetween. In some embodiments, the ablation fluid can be delivered for a period of time of between about 10 seconds and about 5 minutes, inclusive of all values and ranges therebetween, including, for example, 30 seconds. While the cryoablation medium is being delivered, fluids, gases, etc. can be evacuated from the body lumen via an evacuation lumen, as described above. Further details of the process of delivering an ablation medium and evaluating the ablation medium and other fluids from a body lumen are described above with reference to FIG. 6A.

In some embodiments, at least a portion of the dehumidification agent can be delivered to the body lumen with the ablation medium. In other words, 5103 can be at least partially concurrent with 5106. This delivery in concert (e.g., misting or spraying dehumidification agent during delivery of ablation medium) can mitigate transient pressure spikes and excessive dehumidification agent buildup within the body lumen if the combination of fluids is delivered over an extended period of time (e.g., about 10 seconds, about 20 seconds, about 30 seconds, about 40 seconds, about 50 seconds, about 1 minute, about 2 minutes, about 3 minutes, about 4 minutes, about 5 minutes, about 6 minutes, about 7 minutes, about 8 minutes, about 9 minutes, or about 10 minutes, inclusive of all values and ranges therebetween) and the dehumidification agent can be more efficiently evacuated with cryogen gas, reducing the effect of fluid pooling.

Optionally, in some embodiments, the method 5100 can include monitoring one or more parameters during the delivery of the ablation medium and/or the thawing medium, at 5107. In some embodiments, the pressure in the body lumen can be measured. In some embodiments, the temperature in the body lumen and/or components of the ablation catheter can be measured. Further details of an example process of pressure and/or temperature monitoring are described above with reference to FIG. 6B.

At 5108, the method 5100 can include passively or actively thawing the body cavity to body temperature. For instance, during delivering of cryoablation, the temperatures within the body lumen may have dropped to low temperatures. Therefore, thawing may be necessary before another cycle of ablation can be applied to the body cavity. In some embodiments, active thawing can be employed, whereby a thawing fluid is delivered to the body cavity. The thawing fluid can be, for example, air, saline solution, and/or other fluids. In some embodiments, the amount of thawing fluid deployed at 5104 can be at least about 500 μL, at least about 1 mL, at least about 1.5 mL, at least about 2 mL, at least about 2.5 mL, at least about 3 mL, at least about 3.5 mL, at least about 4 mL, or at least about 4.5 mL. In some embodiments, the amount of thawing fluid deployed at 5104 can be no more than about 5 mL, no more than about 4.5 mL, no more than about 4 mL, no more than about 3.5 mL, no more than about 3 mL, no more than about 2.5 mL, no more than about 2 mL, no more than about 1.5 mL, or no more than about 1 mL. Combinations of the above-referenced volumes are also possible (e.g., at least about 500 μL and no more than about 5 mL or at least about 1 mL and no more than about 4 mL), inclusive of all values and ranges therebetween. In some embodiments, passive thawing can be employed, whereby the body lumen passively thaws over time without the injection of a medium into the body lumen.

In some embodiments, the method 5100 includes aspirating residual fluid through the aspiration catheter. Fluids remaining in the body lumen can be expelled from the body lumen. In some embodiments, the cage can be undeployed (i.e., compressed) and the ablation catheter can be withdrawn from the body lumen before aspirating the residual fluid from the body lumen. In some embodiments, the cage can be undeployed and the ablation catheter can be withdrawn from the body lumen after aspirating the residual fluid from the body lumen.

After 5108, the method 5100 can end or can be repeated from 5101, e.g., to apply another cycle of treatment to the body lumen.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Also, various concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within ±10% of the recited value. For example, in some instances, “about 100 [units]” may mean within ±10% of 100 (e.g., from 90 to 110). The terms “about” and “approximately” may be used interchangeably.

Some embodiments described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which may include, for example, the instructions and/or computer code disclosed herein.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

1. A system, comprising:
an outer shaft including a distal end that is disposable within a body cavity;
a first inner shaft disposable within the outer shaft and configured to deliver a cryogenic ablation medium into the body cavity; and
a second inner shaft disposable within the outer shaft and configured to deliver a fluid to the body cavity to dehumidify the body cavity and reduce freezing of fluids during evacuation of the fluids from the body cavity.

2. The system of claim 1, further comprising:
an expandable member disposed on a distal end of the outer shaft, the expandable member configured to retain the distal end of the outer shaft within the body lumen.

3. The system of claim 1, wherein a region between an outer surface of the first inner shaft and an inner surface of the outer shaft forms an evacuation lumen configured to evacuate the fluids from the body cavity.

4. The system of claim 1, further comprising:
a supply of the fluid that is fluidically coupled to the second inner shaft.

5. The system of claim 4, further comprising:
a pressure source in fluidic communication with the supply of the fluid, the pressure source configured to apply a positive pressure to the fluid to cause the delivery of the fluid.

6. The system of claim 1, further comprising:
a supply of the ablation medium in fluidic communication with the first inner shaft.

7. The system of claim 1, further comprising a handle assembly, the handle assembly including:
a supply of the fluid that is fluidically coupled to the second inner shaft; and
a supply of the cryogenic ablation medium in fluidic communication with the first inner shaft.

8. The system of claim 1, wherein the second inner shaft is further configured to aspirate the fluids from the body cavity.

9. The system of claim 8, further comprising a vacuum source coupled to the second inner shaft, the vacuum source configured to apply vacuum pressure to a lumen of the second shaft to aspirate the fluids from the body cavity.

10. The system of claim 9, wherein the vacuum source includes a syringe.

11. The system of claim 9, further comprising a relief lumen coupled to a valve, the valve configured to open in response to the vacuum pressure being above a preset value to allow for pressure release within the lumen.

12. The system of claim 1, wherein at least one of the first and second inner shafts further includes one or more lumens for pressure sensing or temperature sensing.

13. The system of claim 12, wherein the first inner shaft includes:
a pressure sensing lumen; and
a temperature sensing lumen.

14. The system of claim 13, further comprising a pressure sensor in fluidic communication with the pressure sensing lumen.

15. The system of claim 13, further comprising a temperature sensor disposed in the temperature sensing lumen.

16. The system of claim 1, wherein the first inner shaft further comprises one or more heating elements configured to heat one or more lumens of the first inner shaft to reduce freezing.

17. The system of claim 1, wherein the first inner shaft further comprises one or more channels configured to circulate a liquid medium to reduce freezing within one or more lumens of the first inner shaft.

18. The system of claim 1, wherein the second inner shaft is configured to deliver the fluid to the body cavity prior to or during the delivery of the cryogenic ablation medium.

19. A system, comprising:
an outer shaft including a distal end that is disposable within a body cavity;
an inner shaft configured to be inserted into the body cavity via the outer shaft, the inner shaft configured to deliver a cryogenic ablation medium into the body cavity; and
a first lumen configured to deliver a fluid to dehumidify the body cavity and reduce freezing of fluids during evacuation of the fluids from the body cavity.

20. (canceled)

21. (canceled)

22. A system, comprising:
a shaft disposable within a body cavity, the shaft including:
a first lumen configured to deliver a cryogenic ablation medium into the body cavity; and
a second lumen terminating within the body cavity configured to be in fluid communication with a pump to drive a low-freezing point fluid to dehumidify the body cavity and reduce freezing of fluids during evacuation of the fluids from the body cavity.

23. (canceled)

24. (canceled)
